(19) United States
(12) Patent Application Publication
(10) Pub. No.: US 2005/0004200 A1
(43) Pub. Date: Jan. 6, 2005

(54) Pyrazole compounds as integrin receptor antagonists derivatives

(75) Inventors: Thomas D. Penning, Elmhurst, IL (US); Albert Khilevich, Buffalo Grove, IL (US); Barbara B. Chen, Northbrook, IL (US); Preete Gandhi, Bellevue, WA (US); Yaping Wang, Acton, MA (US); Victoria L. Downs, Pinckney, MI (US); Mark L. Boys, Brighton, MI (US); Mark Russell, Gurnee, IL (US); Dale P. Spangler, San Diego, CA (US); Renee M. Huff, Park Ridge, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/741,860

(22) Filed: Dec. 19, 2003

Related U.S. Application Data
(60) Provisional application No. 60/435,168, filed on Dec. 20, 2002.

(51) Int. Cl. A61K 31/416; C 07D 4 5/02; A61K 31/415

(52) U.S. Cl. 514/406; 548/364.1; 548/370.1

(57) ABSTRACT

The present invention relates to a class of compounds represented by the Formula I 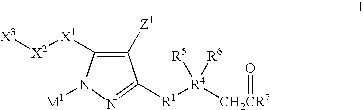
or a pharmaceutically acceptable salt thereof, pharmaceutical compositions comprising compounds of the Formula I, and methods of selectively inhibiting or antagonizing the α_{V}β₃ and/or the α_{V}β₅ integrin without significantly inhibiting the α_{V}β₆ integrin.

### CROSS-REFERENCE TO RELATED APPLICATION

This application claims priority from Provisional Application Ser. No. 60/435,168 filed Dec. 20, 2002, which is hereby incorporated by reference in its entirety.

### FIELD OF INVENTION

The present invention relates to pharmaceutical agents (compounds) that are α_{V}β₃ and/or α_{V}β₅ integrin antagonists and as such are useful in pharmaceutical compositions and in methods for treating conditions mediated by α_{V}β₃ and/or α_{V}β₅ integrins.

### BACKGROUND OF THE INVENTION

The integrin α_{V}β₃ (also known as vitronectin receptor), is a member of the integrin family of heterodimeric transmembrane glycoprotein complexes that mediate cellular adhesion events and signal transduction processes. Integrin α_{V}β₃ is expressed in number of cell types and has been shown to mediate several biologically relevant processes, including adhesion of osteoclasts to the bone matrix, vascular smooth muscle cell migration and angiogenesis.

The integrin α_{V}β₃ has been shown to play a role in various conditions or disease states including tumor metastasis, solid tumor growth (neoplasia), osteoporosis, Paget's disease, humoral hypercalcemia of malignancy, osteopenia, angiogenesis, including tumor angiogenesis, retinopathy including macular degeneration, arthritis, including rheumatoid arthritis, periodontal disease, psoriasis and smooth muscle cell migration (e.g. restenosis arteriosclerosis). The compounds of the present invention are α_{V}β₃ antagonists and can be used, alone or in combination with other therapeutic agents, in the treatment or modulation of various conditions or disease states described above. Additionally, it has been found that such agents would be useful as antivirals, antifungals and antimicrobials.

The integrin α_{V}β₅ plays a role in neovascularization. Therefore the compounds of this invention which act as antagonists of the α_{V}β₅ integrin will inhibit neovascularization and will be useful for treating and preventing angiogenesis metastasis, tumor growth, macular degeneration and diabetic retinopathy.

Antagonists of α_{V}β₃ or dual α_{V}β₃/α_{V}β₅ antagonists can be useful therapeutic agents for treating many pathological conditions, including the treatment or prevention of osteopenia or osteoporosis, or other bone disorders, such as Paget's disease or humoral hypercalcemia of malignancy; neointimal hyperplasia, which can cause artheroscierosis or restenosis after vascular procedures; periodontal disease; treatment and prevention of viral infections or other pathogens; the treatment of neoplasia; pathological angiogenesis or neovascularization such as tumor metastasis, diabetic retinopathy, macular degeneration, rheumatoid arthritis, or osteoarthritis.

Compounds that antagonize the α_{V}β₅ and/or the α_{V}β₃ receptor have been reprinted in the literature. For example, WO 01/96334 provides heteroarylalkanoic acid compounds useful as α_{V}β₃ and/or α_{V}β₅ inhibitors.

### SUMMARY OF THE INVENTION

In general, the present invention is directed to selective integrin receptor antagonist compounds corresponding to Formula (I): 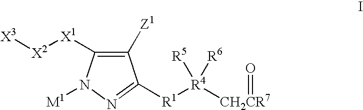
M¹ is hydrocarbyl, substituted hydrocarbyl, heteroaryl, or acyl;
R¹ is —CH(R²)—, —N(R³)—, —O—, —S—, —SO—, —S(O)₂—, —NHS(O)₂—, —S(O)₂NH— or —C(O)—;
R² is hydrogen, hydrocarbyl, substituted hydrocarbyl, alkoxy, or hydroxy, or R² in combination with R⁷ forms a lactone;
R³ is hydrogen, hydrocarbyl, substituted hydrocarbyl, heteroaryl, or acyl;
R⁴ is carbon or nitrogen;
R⁵ is hydrogen, hydrocarbyl, substituted hydrocarbyl, halo or heteroaryl, or R⁵ together with R⁴ and R⁶ form a monocyclic or bicyclic ring system;
R⁶ is an electron pair when R⁴ is nitrogen, or R⁶ is hydrogen, hydrocarbyl, substituted hydrocarbyl, halo or heteroaryl when R⁴ is carbon, or R⁶ together with R⁴ and R⁵ form a monocyclic or bicyclic ring system;
R⁷ is —OR⁸, —SR⁸, —NR⁸R⁹ or R⁷ in combination with R² forms a lactone;
R⁸ is hydrogen, hydrocarbyl, or substituted hydrocarbyl;
R⁹ is hydrogen, hydrocarbyl, substituted hydrocarbyl, alkoxy, substituted alkoxy, or hydroxy;
X¹ is a bond, —O—, —CH₂—, —CH₂O—, —NH—, —C(O)—, —S—, —S(O)—, —CH(OH)—, —S(O)₂—, alkenyl or alkynyl;
X² is linker comprising a chain of 1 to 6 atoms, optionally substituted, optionally unsaturated, selected from the group consisting of C, O, S, and N;
X³ is heterocyclic; and
Z¹ is hydrogen, hydrocarbyl, substituted hydrocarbyl, heteroaryl, hydroxy, or cyano.

The present invention is further directed to a process of treating conditions mediated by α_{V}β₃ and/or α_{V}β₅ integrins in a mammal. The process comprises administering to a mammal in need thereof a therapeutically effective dose of a compound of Formula I.

Other aspects of the invention will be in part apparent and in part pointed out hereinafter.

Definitions

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkyl” embraces linear, cyclic or branched hydrocarbon radicals having one to about twenty carbon atoms or, preferably, one to about twelve carbon atoms. More preferred alkyl radicals are “lower alkyl” radicals having one to about ten carbon atoms. In another embodiment, the alkyl radicals are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “haloalkyl” embraces radicals wherein any one or more of the alkyl carbon atoms is substituted with halo as defined below. Specifically embraced are monohaloalkyl, dihaloalkyl and polyhaloalkyl radicals. A monohaloalkyl radical, for example, can have either an iodo, bromo, chloro or fluoro atom within the radical. Dihalo and polyhaloalkyl radicals can have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkenyl” embraces linear or branched hydrocarbon radicals having at least one carbon-carbon double bond of two to about twenty carbon atoms or, preferably, two to about twelve carbon atoms. More preferred alkyl radicals are “lower alkenyl” radicals having two to about ten carbon atoms. In another embodiment, the alkenyl radicals are lower alkenyl radicals having two to about 6 carbon atoms. Examples of alkenyl radicals include ethenyl, propenyl, allyl, propenyl, butenyl and 4-methylbutenyl. The terms “alkenyl”, “lower alkenyl”, embrace radicals having “cis” and “trans” orientations, or alternatively, “E” and “Z” orientations.

The term “alkynyl” denotes linear or branched carbon or hydrocarbon radicals having two to about twenty carbon atoms or, preferably, two to about twelve carbon atoms. More preferred alkynyl radicals are “lower alkynyl” radicals having two to about ten carbon atoms. In another embodiment, the alkynyl radicals are lower alkynyl radicals having two to about six carbon atoms. Examples of such radicals include propargyl, butynyl, and the like.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The “substituted aryl” moieties described herein are aryl moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “aralkyl” embraces aryl-substituted alkyl radicals such as benzyl, diphenylmethyl, triphenylmethyl, phenylethyl, and diphenylethyl. The “substituted aryl” moieties described herein are aryl moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “amino” is used herein to typically refer to the group —NT²T³, where each of T² and T³ is independently selected from the group consisting of hydrogen, hydrocarbyl, substituted hydrocarbyl, aryl, or heteroaryl. In another embodiment, T² and T³ form a mono or polycyclic amino ring. The term “cyclicamino” embraces saturated heterocyclic radicals having three to eight atoms, at least one of which is nitrogen, but may also contain other heteroatoms such as oxygen, silicon, phosphorous, boron, sulfur, or a halogen.

The term “aminoalkyl” embraces alkyl radicals substituted with one or more amino radicals. More preferred are “lower aminoalkyl” radicals. In general, therefore, aminoalkyl refers to a radical of the Formula: 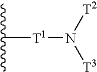
wherein T¹ is alkyl, and T² and T³ are as defined in connection with the definition of amino.

The term “alkylamino” denotes amino groups that have been substituted with one or two alkyl radicals. Preferred is “lower N-alkylamino” radicals having alkyl portions having 1 to 6 carbon atoms. In general, therefore, alkylamino refers to a radical of the Formula: 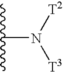
wherein T² and T³ are as defined in connection with the definition of amino. Suitable lower alkylamino may be mono or dialkylamino such as N-methylamino, N-ethylamino, or N,N-dimethylamino.

The term “arylamino” denotes amino groups, which have been substituted with one or two aryl radicals, such as N,N-diphenylamino. The “arylamino” radicals may be further substituted on the aryl ring portion of the radical.

The term “carbonyl”, whether used alone or with other terms, such as “alkoxycarbonyl”, denotes —(C═O)—.

The terms “carboxy” or “carboxyl”, whether used alone or with other terms, such as “carboxyalkyl”, denotes —CO₂H.

The term “carboxyalkyl” embraces alkyl radicals substituted with a carboxy radical. Examples of carboxyalkyl radicals include carboxymethyl, carboxyethyl and carboxypropyl.

The term “halo” means halogens such as fluorine, chlorine, bromine or iodine.

The term “heteroaryl” embraces unsaturated heterocyclyl radicals. Examples of unsaturated heterocyclyl radicals, also termed “heteroaryl” radicals include unsaturated 3 to 8 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 8-membered heteromonocyclic group containing an oxygen atom, for example, pyranyl, furyl, etc.; unsaturated 3 to 8-membered heteromonocyclic group containing a sulfur atom, for example, thienyl, etc.; unsaturated 3- to 8-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 8-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals or a non-aromatic cyclic system. Examples of such fused bicyclic radicals include benzofuran, benzothiophene, and the like.

The “substituted heteroaryl” moieties described herein are heteroaryl moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “heteroatom” shall mean atoms other than carbon and hydrogen.

The term “heterocyclo” and “heterocyclic” embraces optionally substituted saturated, partially unsaturated and unsaturated heteroatom-containing ring-shaped radicals containing 3 to 10 members, including at least 1 carbon atom and up to 9 additional members independently selected from carbon, nitrogen, sulfur and oxygen. This includes, for example, the following structures: 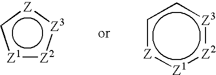
wherein Z, Z¹, Z² or Z³ is C, S, O, or N, with the proviso that one of Z, Z¹, Z² or Z³ is other than carbon, but is not O or S when attached to another Z atom by a double bond or when attached to another O or S atom. Furthermore, optional substituents are understood to be attached to Z, Z¹, Z² or Z³ only when each is C.

Examples of saturated heterocyclyl radicals include saturated 3 to 8-membered heteromonocylic group containing 1 to 4 nitrogen atoms (e.g. pyrrolidinyl, imidazolidinyl, piperidino, piperazinyl, etc.); saturated 3 to 8-membered heteromonocyclic group containing 1 to 2 oxygen atoms and 1 to 3 nitrogen atoms (e.g. morpholinyl, etc.); saturated 3 to 8-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The “substituted heterocyclo” moieties described herein are heterocyclo moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl is optionally substituted with halo, alkyl, alkoxy, haloalkyl and haloalkoxy.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The term substituted hydrocarbyloxy as used herein alone or as part of another group, denotes a substituted hydrocarbyl group as described above bonded through an oxygen linkage (—O—).

The term “hydroxyalkyl” embraces linear or branched alkyl radicals having one to about ten carbon atoms any one of which are optionally substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “lactone” refers to an anhydro cyclic ester produced by intramolecular condensation of a hydroxy acid with the elimination of water.

The term “sulfonamide” or “sulfonamido” refers to a radical of the Formula: 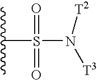
wherein T² and T³ are as defined in connection with the definition of amino.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals are optionally substituted with one or more halo atoms, such as fluoro, chloro or bromo, to provide haloalkylsulfonyl radicals

The term “trifluoroalkyl” refers to an alkyl radical as defined above substituted with three halo radicals as defined above.

The term “methylenedioxy” refers to the radical:

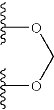

The term “ethylenedioxy” refers to the radical:

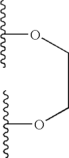

The term “composition” as used herein means a product that results from the mixing or combining of more than one element or ingredient.

The term “pharmaceutically acceptable carrier”, as used herein means a pharmaceutically acceptable material, composition or vehicle, such as a liquid or solid filler, diluent, excipient, solvent or encapsulating material, involved in carrying or transporting a chemical agent.

The term “pharmaceutically acceptable salt” refers to a salt prepared by contacting a compound of Formulae I-IV with an acid whose anion is generally considered suitable for human consumption. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid. Representative salts include the following: benzenesulfonate, hydrobromide and hydrochloride. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. All of the pharmacologically acceptable salts may be prepared by conventional means. (See Berge et al., J. Pharm. Sci. 66(1), 1-19 (1977) for additional examples of pharmaceutically acceptable salts.)

The term “therapeutically effective amount” shall mean that amount of drug or pharmaceutical agent that will elicit the biological or medical response of a tissue, system or animal that is being sought by a researcher or clinician.

As used herein, the term “treatment” is meant the medical management of a subject, e.g. an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to an animal, cell, lysate or extract derived from a cell, or a molecule derived from a cell.

The following is a list of abbreviations and the corresponding meanings as used interchangeably herein:
¹H-NMR=proton nuclear magnetic resonance
AcOH=acetic acid
BOC=tert-butoxycarbonyl
BuLi=butyl lithium
Cat. =catalytic amount
CDI=Carbonyldiimidazole
CH₂Cl₂=dichloromethane
CH₃CN=acetonitrile
CH₃₁=iodomethane
CHN analysis=carbon/hydrogen/nitrogen elemental analysis
CHNCl analysis=carbon/hydrogen/nitrogen/chlorine elemental analysis
CHNS analysis=carbon/hydrogen/nitrogen/sulfur elemental analysis
DEAD=diethylazodicarboxylate
DIAD=diisopropylazodicarboxylate
DI water=deionized water
DMA=N,N-dimethylacetamide
DMAC=N,N-dimethylacetamide
DMF=N,N-dimethylformamide
EDC=1-(3-dimethylaminopropyl)-3-ethylcarbodiimide hydrochloride
Et=ethyl
Et₂O=diethyl ether
Et₃N=triethylamine
EtOAc=ethyl acetate
EtOH=ethanol
FAB MS=fast atom bombardment mass spectroscopy
g=gram(s)
HOBT=1-hydroxybenzotriazole hydrate
HPLC=high performance liquid chromatography
i—Pr=isopropyl
i—Prop=isopropyl
K₂CO₃=potassium carbonate
KMnO₄=potassium permanganate
KOH=potassium hydroxide
KSCN=potassium thiocyanate
L=Liter
LiOH=lithium hydroxide
Me=methyl
MeOH=methanol
mg=milligram
MgSO₄=magnesium sulfate
ml=milliliter
mL=milliliter
MS=mass spectroscopy
NaH—sodium hydride
NaHCO₃=sodium bicarbonate
NaOH=sodium hydroxide
NaOMe=sodium methoxide
NH₄^{+HCO}₂⁻=ammonium formate
NMR=nuclear magnetic resonance
Pd=palladium
Pd/C=palladium on carbon
Ph=phenyl
Pt=platinum
Pt/C=platinum on carbon
RPHPLC=reverse phase high performance liquid chromatography
RT=room temperature
t-BOC=tert-butoxycarbonyl
TFA=trifluoroacetic acid
THF=tetrahydrofuran
TLC—thin layer chromatography
TMS=trimethylsilyl
Δ=heating the reaction mixture

The compounds as shown above can exist in various isomeric forms and all such isomeric forms are meant to be included. Tautomeric forms are also included as well as pharmaceutically acceptable salts of such isomers and tautomers.

In the structures and Formulae herein, a bond drawn across a bond of a ring can be to any available atom on the ring.

### DETAILED DESCRIPTION OF THE PREFERRED EMBODIMENTS

In one embodiment, the compounds of the present invention correspond to Formula (I) 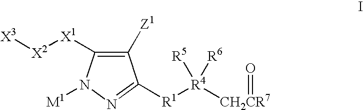
wherein:
M¹ is selected from the group consisting of heteroaryl, acyl, and optionally substituted hydrocarbyl, wherein the optional substituents are selected from the group consisting of alkyl, halo, haloalkyl, hydroxy, alkoxy, amino, alkylamino, dialkylamino, cyano, acyl, —S—, —S0-, —SO₂—, sulfonamido, aryl, and heteroaryl;
R¹ is selected from the group consisting of —CH(R²)—, —N(R³)—,
—O—, —S—, —S(O)₂—, —NHS(O)₂—, —S(O)₂NH— and —C(O)—;
R² is selected from the group consisting of hydrogen, hydroxy, and optionally substituted hydrocarbyl or alkoxy, wherein the optional substituents are selected from the group consisting of alkyl, halogen, hydroxy, alkoxy, amino, alkylamino, dialkylamino, cyano, acyl, —S—, —S0-, —SO₂—, sulfonamido, aryl, and heteroaryl, or R² in combination with R⁷ forms a lactone;
R³ is selected from the group consisting of hydrogen and optionally substituted hydrocarbyl, heteroaryl, and acyl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
R⁴ is carbon or nitrogen;
R⁵ is selected from the group consisting of hydrogen, halo, hydrocarbyl, and optionally substituted heteroaryl, wherein the optional substituents are selected from the group consisting of alkyl, halogen, hydroxy, alkoxy, alkoxyalkyl, amino, alkylamino, dialkylamino, cyano, acyl, —S—, —SO—, —SO₂—, sulfonamido, heteroaryl, and optionally substituted aryl, wherein the optional substituent is halo, or R⁵ together with R⁴ and R⁶ forms a heterocycle or aryl ring;
R⁶ is an electron pair when R⁴ is nitrogen, or R⁶ is heterocyclo when R⁴ is carbon, or R⁶ is hydrogen, halo, or optionally substituted hydrocarbyl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl, or R⁶ together with R⁴ and R⁵ forms a heterocycle or aryl ring;
R⁷ is selected from the group consisting of —OR⁸, —SR⁸, and —NR⁸R⁹;
R⁸ is selected from the group consisting of hydrogen and optionally substituted hydrocarbyl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
R⁹ is selected from the group consisting of hydrogen, hydroxy, and optionally substituted hydrocarbyl or alkoxy, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
X¹ is selected from the group consisting of —O—, —CH₂—, —CH₂O—, —NH—, —C(O)—, —S—, —S(O)—, CH(OH)—, —S(O)₂—, alkenyl, and alkynyl;
X² is a linker comprising a chain of 1 to 5 atoms, optionally substituted, selected from the group consisting of C, O, S and N;
X³ is heterocyclic; and
Z¹ is selected from the group consisting of hydrogen, hydroxy, cyano, and optionally substituted hydrocarbyl or heteroaryl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl.

In another embodiment for compounds having Formula I, Ml is alkyl or substituted alkyl such as methyl, hydroxymethyl, carboxymethyl, trifluoroethyl, —(CH₂)ₘCN wherein m is 1-4, or —(CH₂)ₘCOM² wherein m is 1-4 and M² is hydroxy, alkoxy, alkyl, amino, alkylamino, dialkylamino, or arylamino. In another embodiment M¹ is aryl, substituted aryl, or heteroaryl such as phenyl. In the previous two embodiments, substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl.

In still another embodiment for compounds having Formula I, Z, is alkyl or substituted alkyl. In yet another embodiment, Z¹ is aryl, substituted aryl, or heteroaryl. In the previous two embodiments, substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl. In a further embodiment, Z¹ is hydrogen.

In yet another embodiment for compounds having Formula I, X² is a carbon chain comprising 1 to 3 carbon atoms. In another embodiment, X² is optionally substituted. In the previous embodiment, substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl. In still another embodiment, X² comprises a carbon-carbon unsaturated bond.

In a further embodiment for compounds having Formula I, X³ is selected from the group consisting of: 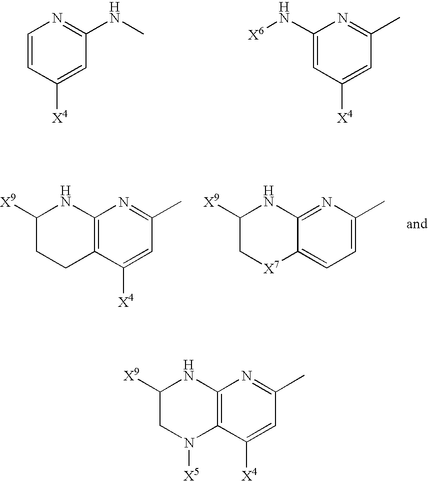
wherein:
X⁴ is hydrogen, hydroxy, alkoxy, hydrocarbyl, substituted hydrocarbyl, amino, alkylamino, dialkylamino, cyclicamino, heterocyclo, or —NHSO₂R¹¹ wherein R¹¹ is alkyl or aryl;
X ⁵, X⁶, and X⁸ are independently hydrogen, hydrocarbyl, substituted hydrocarbyl, or heterocyclo;
X⁷ is —CH₂—, —CH₂O—, —OCH₂— —S—, —SO—, —SO₂—, —O—, —C(O)—, —CH(OH)—, —NH—, or —NX⁸; and
X⁹ is ═O, or —OH.

In another embodiment for compounds having Formula I, X¹ is oxygen. In a further embodiment, X¹ is —S—, —SO—, or —SO₂—. In still another embodiment, X¹ is —NH—. In yet another embodiment X¹ is —CH₂—.

In another embodiment for compounds having Formula I, R¹ is —CH(R²)— wherein R² is hydrogen, hydroxy, or alkoxy. In yet another embodiment, R¹ is —N(R³)— wherein R³ is selected from the group consisting of hydrogen, alkyl, substituted alkyl, substituted aryl, and heteroaryl.

In a further embodiment, R¹ is —S—, —SO—, —SO₂—, NHS(O)₂—, or —S(O)₂NH—.

In still further embodiment, R¹ is oxygen.

In another embodiment for compounds having Formula I, R⁴ is carbon. In yet another embodiment, R⁴ is nitrogen.

In a further embodiment for compounds having Formula I, R⁵ is hydrogen. In still another embodiment, R⁵ is alkyl or substituted alkyl. In yet another embodiment, R⁵ is aryl or heteroaryl.

In another embodiment for compounds having Formula I, R⁶ is an electron pair. In yet another embodiment, R⁶ is selected from the group consisting of hydrogen, alkyl, substituted alkyl, aryl, and heteroaryl.

In a further embodiment for compounds having Formula I, R⁷ is hydroxy or alkoxy.

The present invention is further directed to compounds that correspond to Formula (II). 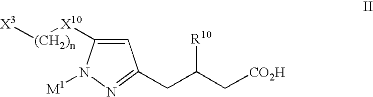
wherein:
X³ is heterocyclic;
n is 0-3;
X¹⁰ is —O—, —S—, —SO—, —SO₂—, or —CH₂—;
M¹ is hydrocarbyl, substituted hydrocarbyl, or heteroaryl; and
R¹⁰ is aryl, substituted aryl, aralkyl, substituted aralkyl, heteroaralkyl, substituted heteroaralkyl, or heteroaryl.

In one embodiment for compounds having Formula II, M¹ is alkyl or substituted alkyl such as methyl, hydroxymethyl, carboxymethyl, trifluoroethyl, —(CH₂)ₘCN wherein m is 1-4, or —(CH₂)ₘCOM² wherein m is 1-4 and M² is hydroxy, alkoxy, alkyl, amino, alkylamino, dialkylamino, or arylamino. In another embodiment M¹ is aryl, substituted aryl, or heteroaryl such as phenyl.

In a further embodiment for compounds having Formula II, X³ is selected from the group consisting of: 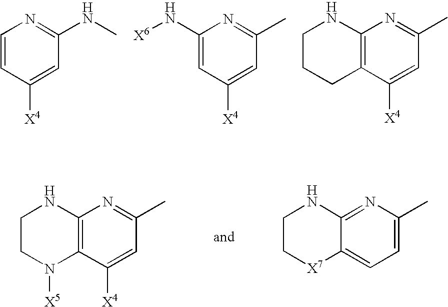
wherein:
X⁴ is hydrogen, hydroxy, alkoxy, hydrocarbyl, substituted hydrocarbyl, amino, or heterocyclo;
X⁵, X⁶, and X⁸ are independently hydrogen, hydrocarbyl, substituted hydrocarbyl, or heterocyclo; and
X⁷ is —CH₂—, —CH₂O—, —OCH₂— —S—, —O—, —C(O)—, —CH(OH)—, —NH—, or —NX⁸.

In another embodiment for compounds having Formula II, R¹⁰ is aryl, substituted aryl, or heteroaryl. In a further embodiment, R¹⁰ is monocyclic. In still further embodiment, R¹⁰ is bicyclic. In yet another embodiment, R¹⁰ optionally contains 0 to 5 heteroatoms. In the previous four embodiments, substituents are selected from the group consisting of alkyl, haloalkyl, aryl, heteroaryl, halogen, alkoxyalkyl, aminoalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)ₘCOR wherein m is 0-2 and R is hydroxy, alkoxy, alkyl and amino.

In another embodiment for compounds having Formula II, the compound is the “R” or “S” isomer.

The present invention is further directed to compounds that correspond to Formula (III). 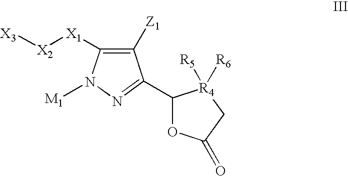
wherein:
M¹ is hydrocarbyl, substituted hydrocarbyl, heteroaryl, or acyl;
R⁴ is carbon or nitrogen;
R⁵ is hydrogen, hydrocarbyl, substituted hydrocarbyl, halo or heterocyclo, or R⁵ together with R⁴ and R⁶ form a monocyclic or bicyclic ring system;
R⁶ is an electron pair when R⁴ is nitrogen, or R⁶ is hydrogen, hydrocarbyl, substituted hydrocarbyl, halo or heterocyclo when R⁴ is carbon, or R⁶ together with R⁴ and R⁵ form a monocyclic or bicyclic ring system;
X¹ is a bond, —O—, —CH₂—, —CH₂O—, —NH—, —C(O)—, —S—, —S(O)—, —CH(OH)—, or —S(O)₂—;
X² is linker comprising a chain of 1 to 6 atoms, optionally substituted, optionally unsaturated, selected from the group consisting of C, O, S and N;
X³ is heterocyclic; and
Z¹ is hydrogen, hydrocarbyl, substituted hydrocarbyl, heteroaryl, hydroxy, or cyano.

In one embodiment for compounds having Formula III, M¹ is alkyl or substituted alkyl such as methyl, hydroxymethyl, carboxymethyl, trifluoroethyl, —(CH₂)ₘCN wherein m is 1-4, or —(CH₂)ₘCOM² wherein m is 1-4 and M² is hydroxy, alkoxy, alkyl, amino, alkylamino, dialkylamino, or arylamino. In another embodiment M¹ is aryl, substituted aryl, or heteroaryl such as phenyl. In the previous two embodiments, substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl.

In another embodiment for compounds having Formula III, Z¹ is alkyl or substituted alkyl. In yet another embodiment, Z¹ is aryl, substituted aryl, or heteroaryl. In the previous two embodiments, substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl. In a further embodiment, Z¹ is hydrogen.

In another embodiment for compounds having Formula III, X² is a carbon chain comprising 1 to 3 carbon atoms. In yet another embodiment, x² is optionally substituted. In the previous embodiment, substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl. In still another embodiment, X² comprises a carbon-carbon unsaturated bond.

In a further embodiment for compounds having Formula II, X³ is selected from the group consisting of: 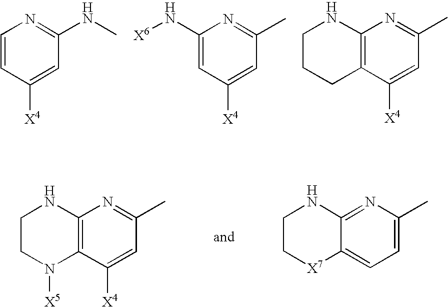
wherein:
X⁴ is hydrogen, hydroxy, alkoxy, hydrocarbyl, substituted hydrocarbyl, amino, alkylamino, dialkylamino, cyclicamino, heterocyclo, or —NHSO₂R¹¹ wherein R¹¹ is alkyl or aryl;
X⁵, X⁶, and x⁸ are independently hydrogen, hydrocarbyl, substituted hydrocarbyl, or heterocyclo; and
X⁷ is —CH₂—, —CH₂O—, —OCH₂— —S—, —SO—, —SO₂—, —O—, —C(O)—, —CH(O H)—, —NH—, or —NX⁸.

In another embodiment for compounds having Formula II, X¹ is oxygen. In a further embodiment, X¹ is —S—, —SO—, or —SO₂—. In still another embodiment, X¹ is —NH—. In yet another embodiment X¹ is —CH₂—.

In another embodiment for compounds having Formula III, R⁴ is carbon. In yet another embodiment, R⁴ is nitrogen.

In a further embodiment for compounds having Formula III, R⁵ is hydrogen. In another embodiment, R⁵ is alkyl or substituted alkyl. In yet another embodiment, R⁵ is aryl or heteroaryl.

In another embodiment for compounds having Formula III, R⁶ is an electron pair. In yet another embodiment, R⁶ is selected from the group consisting of hydrogen, alkyl, substituted alkyl, aryl, and heteroaryl.

In a further embodiment for compounds having Formula III, R⁴, R⁵, and R⁶ form a ring. In yet another embodiment, the ring formed by R⁴, R⁵, and R⁶ is monocyclic. In still another embodiment, the ring formed by R⁴, R⁵, and R⁶ is bicyclic.

The present invention is further directed to compounds that correspond to Formula (IV). 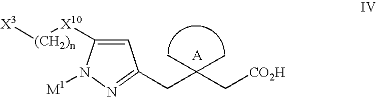
wherein:
X³ is heterocyclic;
n is 0-3;
X¹⁰ is —O—, —S—, —SO—, —SO₂—, or —CH₂—;
M¹ is hydrocarbyl, substituted hydrocarbyl, or heteroaryl; and
A is aryl, substituted aryl, or heteroaryl.

In one embodiment for compounds having Formula IV, M¹ is alkyl or substituted alkyl such as methyl, hydroxymethyl, carboxymethyl, trifluoroethyl, —(CH₂)ₘCN wherein m is 1-4, or —(CH₂)ₘCOM² wherein m is 1-4 and M² is hydroxy, alkoxy, alkyl, amino, alkylamino, dialkylamino, or arylamino. In another embodiment M¹ is aryl, substituted aryl, or heteroaryl such as phenyl.

In a further embodiment for compounds having Formula IV, X³ is selected from the group consisting of: 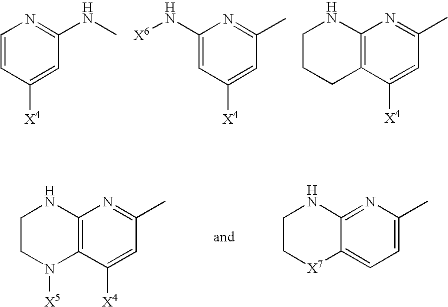
wherein:
X⁴ is hydrogen, hydroxy, alkoxy, hydrocarbyl, substituted hydrocarbyl, amino, or heterocyclo;
X⁵, X⁶, and X⁸ are independently hydrogen, hydrocarbyl, substituted hydrocarbyl, or heterocyclo; and
X⁷ is —CH₂—, —CH₂O—, —OCH₂—, —S—, —O—, —C(O)—, —CH(OH)—, —NH—, or —NX⁸.

In another embodiment for compounds having Formula IV, A is aryl, substituted aryl, or heteroaryl. In a further embodiment, A is monocyclic. In still further embodiment, A is bicyclic. In yet another embodiment, A optionally contains 0 to 3 heteroatoms. In the previous four embodiments, substituents are selected from the group consisting of alkyl, haloalkyl, aryl, heteroaryl, halogen, alkoxyalkyl, aminoalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)ₘCOR wherein m is 0-2 and R is hydroxy, alkoxy, alkyl and amino.

The present invention includes within its scope prodrugs of the compounds of this invention. Any compound corresponding to any of Formulae (I)-(IV), having one or more prodrug moieties as part of the molecule, can be converted under physiological conditions to the biologically active drug by a number of chemical and biological mechanisms. In general terms, these prodrug conversion mechanisms are hydrolysis, reduction, oxidation, and elimination.

In general, such prodrugs will be functional derivatives of the compounds of this invention which are readily convertible in vivo into the required compound. For example, prodrugs of a carboxylic acid include an ester, an amide, or an ortho-ester. Thus, in the methods of treatment of the present invention, the term “administering” shall encompass the treatment of the various conditions described with the compound specifically disclosed or with a compound which may not be specifically disclosed, but which converts to the compound of Formula I in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

A further aspect of the invention encompasses conversion of the prodrug to the biologically active drug by elimination of the prodrug moiety. Generally speaking, in this embodiment the prodrug moiety is removed under physiological conditions with a chemical or biological reaction. The elimination results in removal of the prodrug moiety and liberation of the biologically active drug. Any compound of the present invention corresponding to Formulae (I)-(IV) may undergo any combination of the above detailed mechanisms to convert the prodrug to the biologically active compound. For example, a particular compound may undergo hydrolysis, oxidation, elimination, and reduction to convert the prodrug to the biologically active compound. Equally, a particular compound may undergo only one of these mechanisms to convert the prodrug to the biologically active compound.

The compounds of the present invention can have chiral centers and occur as racemates, racemic mixtures, diastereomeric mixtures, and as individual diastereomers or enantiomers, with all isomeric forms included in the present invention. Therefore, where a compound is chiral, the separate enantiomers or diastereomers, substantially free of the other, are included within the scope of the present invention; further included are all mixtures of the enantiomers or diastereomers. The compounds of the present invention can exist in tautomeric, geometric or stereoisomeric forms. The present invention contemplates all such compounds, including cis- and trans-geometric isomers, E- and Z-geometric isomers, R- and S-enantiomers, diastereomers, d-isomers, I-isomers, the racemic mixtures thereof and other mixtures thereof, as falling within the scope of compounds having any of Formulae (I)-(IV). The terms “cis” and “trans”, as used herein, denote a form of geometric isomerism in which two carbon atoms connected by a double bond will each have a hydrogen atom on the same side of the double bond (“sis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present. Also included within the scope of the invention are polymorphs, or hydrates or other modifiers of the compounds of invention.

Moreover, the family of compounds or isomers having any of Formulae (I)-(IV) also include the pharmaceutically acceptable salts thereof. Pharmaceutically acceptable salts of such tautomeric, geometric or stereoisomeric forms are also included within the invention. The term “pharmaceutically-acceptable salt” embraces salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, and galacturonic acid. Suitable pharmaceutically-acceptable base addition salts of the compounds include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compound by reacting, for example, the appropriate acid or base with the selected compound of any of Formulae (I)-(IV).

The present invention also comprises a pharmaceutical composition comprising a therapeutically effective amount of the compound of the invention in association with at least one pharmaceutically acceptable carrier, adjuvant or diluent. Pharmaceutical compositions of the present invention can comprise the active compounds of Formulae (I)-(IV) in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended.

The compounds of this invention include 1) α_{V}β₃ integrin antagonists; or 2) α_{V}β₅ integrin antagonists; or 3) mixed or dual α_{V}β₃/α_{V}β₅ antagonists. The present invention includes compounds which inhibit the respective integrins and also includes pharmaceutical compositions comprising such compounds. The present invention further provides for methods for treating or preventing conditions mediated by the α_{V}β₃ and/or α_{V}β₅ receptors in a mammal in need of such treatment comprising administering a therapeutically effective amount of the compounds of the present invention and pharmaceutical compositions of the present invention. Administration of such compounds and compositions of the present invention inhibits angiogenesis, tumor metastasis, tumor growth, skeletal malignancy of breast cancer, osteoporosis, Paget's disease, humoral hypercalcemia of malignancy, retinopathy, macular degeneration, arthritis including rheumatoid, periodontal disease, smooth muscle cell migration, including restenosis and artherosclerosis, and microbial or viral diseases. The compounds of the present invention can be used, alone or in combination with other therapeutic agents, in the treatment or modulation of various conditions or disease states described above.

In order to prevent bleeding side effects associated with the inhibition of α_{IIb}β₃, it would be beneficial to have a high selectivity ratio of α_{V}β₃ and α_{V}β₅ over α_{IIb}β₃. The compounds of the present invention include selective antagonists of α_{V}β₃ over α_{IIb}β₃. The compounds of the present invention further show greater selectivity for the α_{V}β₃ and/or α_{V}β₅ integrin than for the α_{V}β₆ integrin. It has been found that the selective antagonism of the α_{V}β₃ integrin is desirable in that the α_{V}β₆ integrin plays a role in normal physiological processes of tissue repair and cellular turnover that routinely occur in the skin and pulmonary tissue, and the inhibition of this function can be deleterious (Huang et al., Am J Respir Cell Mol Biol 1998, 19(4): 636-42). Therefore, compounds of the present invention which selectively inhibit the α_{V}β₃ integrin as opposed to the α_{V}β₆ integrin have reduced side effects associated with inhibition of the α_{V}β₆ integrin.

For the selective inhibition or antagonism of α_{V}β₃ and/or α_{V}β₅ integrins, compounds of the present invention may be administered orally, parenterally, or by inhalation spray, or topically in unit dosage Formulations containing conventional pharmaceutically acceptable carriers, adjuvants and vehicles. The term parenteral as used herein includes, for example, subcutaneous, intravenous, intramuscular, intrasternal, transmuscular infusion techniques or intraperitonally.

The compounds of the present invention are administered by any suitable route in the form of a pharmaceutical composition adapted to such a route, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

Accordingly, the present invention provides a method of treating conditions mediated by selectively inhibiting or antagonizing the α_{V}β₃ and/or α_{V}β₅ cell surface receptor which method comprises administering a therapeutically effective amount of a compound selected from the class of compounds depicted in the above Formulae, wherein one or more compound is administered in association with one or more non-toxic, pharmaceutically acceptable carriers and/or diluents and/or adjuvants (collectively referred to herein as “carrier” materials) and if desired other active ingredients. More specifically, the present invention provides a method for selective antagonism of the α_{V}β₃ and/or α_{V}β₅ cell surface receptors over α_{IIb}β₃ or α_{V}β₆ integrin receptors. In one embodiment, the present invention provides a method for inhibiting bone resorption, treating osteoporosis, inhibiting humoral hypercalcemia of malignancy, treating Paget's disease, inhibiting tumor metastasis, inhibiting neoplasia (solid tumor growth), inhibiting angiogenesis including tumor angiogenesis, treating retinopathy including macular degeneration and diabetic retinopathy, inhibiting arthritis, psoriasis and periodontal disease, and inhibiting smooth muscle cell migration including restenosis. In another embodiment, the present invention provides a method for treating osteoporosis. In yet another embodiment, the present invention provides a method for treating tumor metastasis. In another embodiment, the present invention provides a method of treating inappropriate angiogenesis.

Based upon standard laboratory experimental techniques and procedures well known and appreciated by those skilled in the art, as well as comparisons with compounds of known usefulness, the compounds of Formula I can be used in the treatment of patients suffering from the above pathological conditions. One skilled in the art will recognize that selection of the most appropriate compound of the invention is within the ability of one with ordinary skill in the art and will depend on a variety of factors including assessment of results obtained in standard assay and animal models.

Treatment of a patient afflicted with one of the pathological conditions comprises administering to such a patient an amount of compound of the Formula I which is therapeutically effective in controlling the condition or in prolonging the survivability of the patient beyond that expected in the absence of such treatment. As used herein, the term “inhibition” of the condition refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

As stated previously, the compounds of the invention can be used in a variety of biological, prophylactic or therapeutic areas. It is contemplated that these compounds are useful in prevention or treatment of any disease state or condition wherein the α_{V}β₃ and/or α_{V}β₅ integrin plays a role.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 1.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 200 or 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regiment.

For administration to a mammal in need of such treatment, the compounds in a therapeutically effective amount are ordinarily combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulphuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The pharmaceutical compositions useful in the present invention may be subjected to conventional pharmaceutical operations such as sterilization and/or may contain conventional pharmaceutical adjuvants such as preservatives, stabilizers, wetting agents, emulsifiers, buffers, etc.

In general, the compounds of the present invention may be synthesized as described below and as depicted in reaction scheme 1. 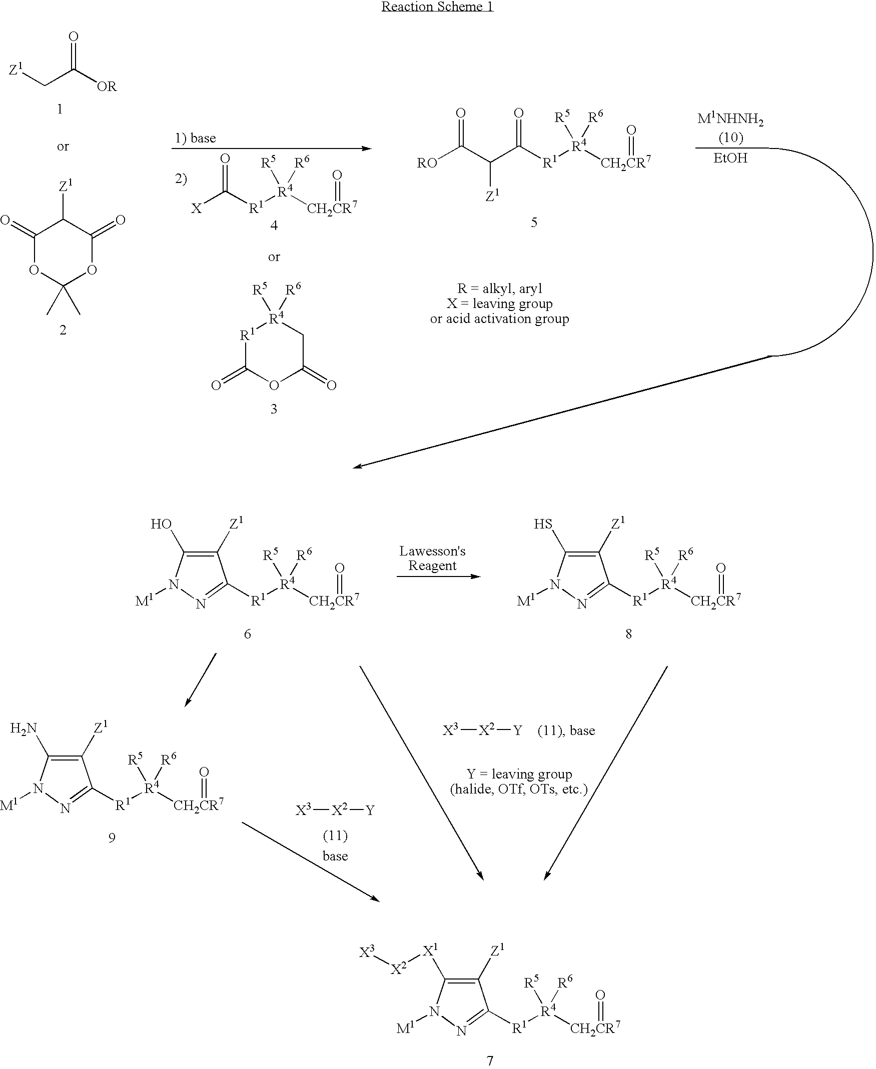
wherein R¹-R⁷, Xl —X³, and Z¹ are as defined in connection with Formula 1.

An optionally substituted acetic ester 1 or Meldrum's acid derivative 2 is reacted with a base such as lithium di-isopropylamide or lithium hexamethyldisilazide in a solvent such as THF or Et₂O to form the corresponding enolate. A cyclic anhydride 3 or acid halide or activated acid 4 is added to give a 1,3-ketoester 5. This is condensed with a substituted hydrazine 10 in a solvent such as ethanol to give hydroxypyrazole 6. O-alkylation of the hydroxypyrazole with X³—X²—Y (11), where Y is an appropriate leaving group such as halide or alkyl- or aryl- sulfonate gives coupled product 7 (X¹=0). Alternatively, when Y=—OH, the hydroxypyrazole could be reacted under Mitsunobu conditions to give 7 (X¹=0). Hydroxypyrazole 6 can be converted the thiol derivative 8 using reagents such as Lawesson's reagent (2,4-bis(4-methoxyphenyl)-1,3-dithia-2,4-diphosphetane-2,4-disulfide). Alkylation as described above gives 7 (X¹=S). Further oxidation using reagents such as m-chloroperbenzoic acid or OXONE gives 7 (X¹=SO and SO₂). Hydroxypyrazole 6 could also be converted to aminopyrazole 9 and alkylated as described above to give 7 (X¹=NH). In all cases, the final step is reaction of 7 under either basic or acidic conditions to give 7 where R⁷=—OH.

### EXAMPLES

### Example 1

### 3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoic acid.

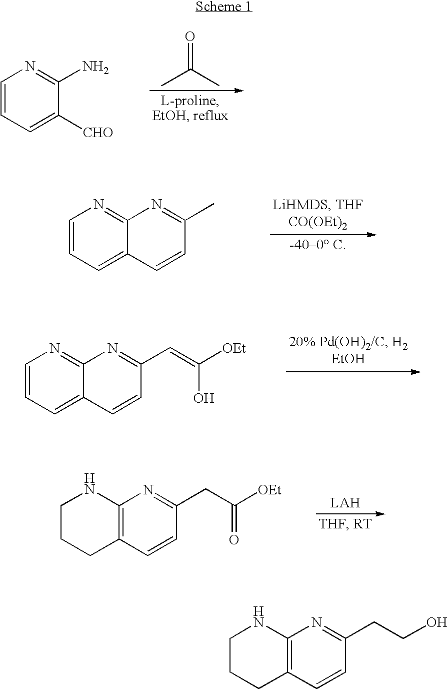

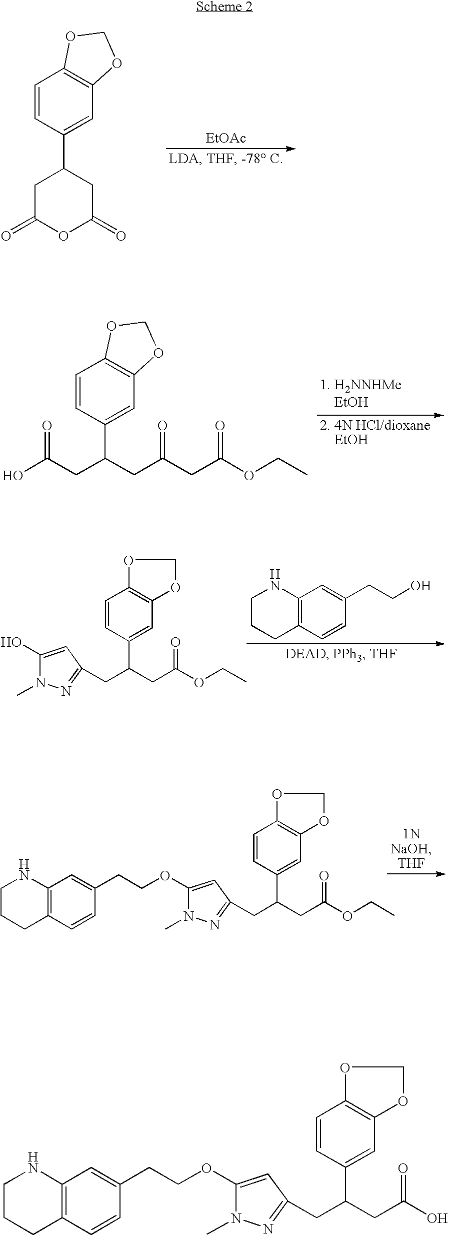

Step 1. Synthesis of 2-methyl-1,8-naphthyridine.

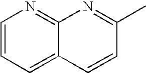

To 2-amino-3-nicotinaldehyde (50.0 g, 0.41 mol) in EtOH (600 mL) was added L-proline (51 g, 0.45 mol) and acetone (90 mL, 1.23 mol). The reaction mixture was refluxed overnight. The reaction mixture was cooled to room temperature and the white solid filtered. The filtrate was concentrated to a yellowish solid, redissolved in CH₂Cl₂ (500 mL), and the insolubles filtered. The filtrate was washed with water (2×100 mL), the organic layer was separated and the aqueous layers combined and washed with CH₂Cl₂ (4×75 mL). The organic layers were combined, washed with brine, dried over Na₂SO₄ and concentrated to a yellow solid (57.2 g, 0.40 mol, 97%).

Step 2. Synthesis of (E)-1-ethoxy-2-(1,8-naphthyridin-2-yl)ethanol.

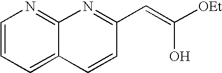

To the product from step 1, (81.5 g, 0.57 mol) in anhydrous THF (1.9 L) at −40° C. under Ar gas was added lithium bis(trimethylsilyl)amide (1M in THF, 1.2 L, 1.2 mol). After stirring for 30 min at −40° C., diethylcarbonate (72.5 mL, 0.60 mol) was added. The temperature of the reaction mixture was warmed up to 0° C. and stirred for 2 h. The reaction mixture was quenched into saturated aq. NH₄Cl (700 mL) and the THF removed under reduced pressure. The resulting mixture was extracted with EtOAc (3×700 mL). The organic layers were combined, washed with brine, dried over Na₂SO₄, and concentrated under reduced pressure. The resulting residue was purified by flash column chromatography using 50% EtOAc/hexane to give a yellow solid (81.2 g, 0.38 mol, 66%). ¹H NMR (400 MHz, DMSO-d6) δ 1.22 (t, 3H), 4.11(q, 2H), 4.89 (s, 1H), 6.78 (d, 1H), 7.15 (dd, 1H), 7.47 (d, 1H), 7.80 (d, 1H), 8.36 (d, 1H), 11.8 (bs, 1H). LC-MS (MH⁺)=217.

Step 3. Synthesis of ethyl 5,6,7,8-tetrahydro-1,8-naphthyridin-2-ylacetate.

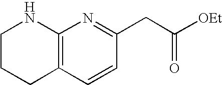

Compound from step 2, (51.4 g, 0.24 mol) in EtOH was hydrogenated using 20% Pd(OH)₂/C at room temperature under a pressure of 5 psi. After 2 h, the reaction was complete. The Pd(OH)₂/C was filtered and the filtrate concentrated to a yellow solid (50.3 g, 0.23 mol, 96%). ¹H NMR (400 MHz, DMSO-d6) δ 1.17 (t, 3H), 1.74 (m, 2H), 2.61 (t, 2H), (3.23, 2H), 3.47 (s, 2H), 4.04 (q, 2H), 6.32 (d, 1H), 6.41 (bs, 1H), 7.07 (d, 1H). LC-MS (MH⁺)=221.

Step 4. Synthesis of 2-(5,6,7,8-tetrahydro-1,8-naphthyridin-2-yl)ethanol.

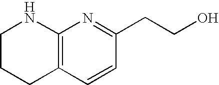

To anhydrous THF (910 mL) under Ar gas at room temperature was added a 1M solution of lithium aluminum hydride in THF (910 mL, 0.91 mol). The temp of the reaction mixture was lowered to 15 IC and a solution of product from step 3, (50.3 g, 0.23 mol) in anhydrous THF (500 mL) was slowly added over 30 min. The resulting reaction was stirred at room temperature for 3.5 h. The temperature was lowered to 0 IC and the reaction was slowly quenched with brine (260 mL). Additional THF (300 mL) was added during the quench to break-up the emulsions. After complete addition of brine, the reaction mixture was stirred at RT overnight. Na₂SO₄ was added and the mixture stirred for 15 min and filtered. The residue was washed with EtOAc (3×300). The organics were combined, concentrated to about 1.5 L, dried with Na₂SO₄, and concentrated under reduced pressure. The resulting residue was purified by flash column chromatography using 100% EtOAc, followed by 5% MeOH/EtOAc as eluents. The desired product was obtained as solid (34.9 g, 85%).

Step 5. Synthesis of 3-(1,3-benzodioxol-5-yl)-7-ethoxy-5,7-dioxoheptanoic acid.

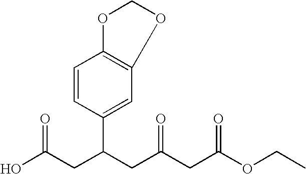

To a solution of anhydrous EtOAc (4.38 mL, 44.8 mmol) in anhydrous THF (25 mL) at −78° C. under Ar gas was slowly added lithium diisopropylamide (2M in heptane /THF/ ethylbenzene, 22.4 mL, 44.8 mmol). The resulting solution was stirred at −78° C. for 25 min and added drop wise via cannula to a solution of the anhydride A (5.0 g, 21.3 mmol) (WO 0196334 A2) in anhydrous THF (170 mL) at −78° C. under Ar gas. The reaction mixture was stirred at −78° C. for 1.5 h. The reaction mixture was quenched with 2N HCl in ether (80 mL) and allowed to warm up to room temperature. To the reaction mixture was added water (100 mL) and extracted with EtOAc (3×100 mL). The organic layers were combined, washed with brine, dried over Na₂SO₄, and concentrated under reduced pressure. The resulting residue was purified by flash column chromatography using 40% EtOAc/hexane to give a white solid (5.61 g, 17.4 mmol, 82%). ¹H NMR (400 MHz, CDCl₃) δ 1.25 (t, 3H), 2.55-2.73 (m, 2H), 2.90 (m, 2H), 3.34 (s, 2H), 3.60 (m, 1H), 4.15 (q, 2H), 5.93 (s, 2H), 6.70 (m, 3H). LC-MS (M+Na)=345.

Step 6. Synthesis of ethyl 3-(1,3-benzodioxol-5-yl)-4-(5-hydroxy-1-methyl-1H-pyrazol-3-yl)butanoate.

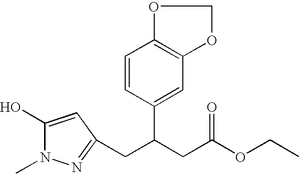

Methyl hydrazine (165 μL, 3.1 mmol) was added drop wise to a stirred solution of the product of step 5 (900 mg, 2.8 mmol) in absolute ethanol (40 mL) at 40° C. After complete addition, the reaction mixture was refluxed for 5 h. The solvent was removed under reduced pressure. The resulting residue was dissolved in absolute ethanol (10 mL) and 4N HCl in dioxane (10 mL) was added. The reaction mixture was stirred at room temperature overnight. The solvent was removed under reduced pressure and the residue purified by flash column chromatography using 5% MeOH/EtOAc as eluent. Obtained was a yellow foam-solid (310 mg, 0.93 mmol, 30%). ¹H NMR (400 MHz, DMSO-d6) δ 1.04 (t, 3H), 2.47 (m, 1H), 2.61 (m, 3H), 3.20 (m, 1H), 3.40 (s, 3H), 3.92 (dq, 2H), 5.02 (s, 1H), 5.95 (s, 2H), 6.65 (dd, 1H), 6.77 (d, 1H), 6.85 (d, 1H), 10.51 (bs, 1H). LC-MS (MH⁺)=333.

Step 7. Synthesis of ethyl 3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoate.

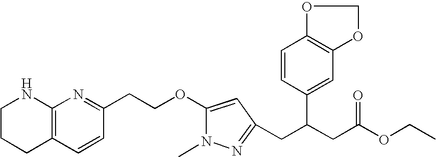

To the product of step 4 (176 mg, 0.99 mmol), the product of step 6 (300 mg, 0.90 mmol), and triphenylphosphine (283 mg, 1.1 mmol) in anhydrous THF (3.5 mL) under Ar gas at room temperature was added diethyl azodicarboxylate (170 μL, 1.1 mmol). The reaction mixture was stirred overnight. The reaction mixture was quenched into saturated aqueous NH₄Cl (5 mL) and extracted with EtOAc (3×5 mL). The organic layers were combined, washed with brine, dried over Na₂SO₄, and concentrated under reduced pressure. The residual oil was purified by flash column chromatography using 3% MeOH/EtOAc as eluent. Obtained was an oil (135 mg) containing a triphenylphosphine oxide impurity. LC-MS (MH⁺)=493.

Step 8. Synthesis of 3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoic acid.

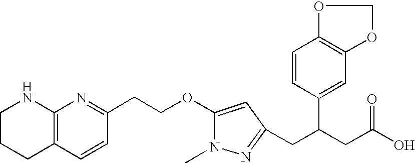

To the product of step 7 (135 mg) in THF (3 mL) was added 1N NaOH (3 mL). The reaction mixture was heated at 50° C. for 5 h and allowed to cool to room temperature overnight. The reaction mixture was acidified, concentrated, and purified by reverse phase HPLC using (H₂O/TFA)/CH₃CN as eluent (2.5 mL TFA in 4 L H₂O). Obtained was the desired product (68 mg). ¹H NMR (400 MHz, DMSO-d6) δ 1.83 (m, 2H), 2.38-2.60 (m, 2H), 2.64 (m, 2H), 2.75 (t, 2H), 3.10 (t, 2H), 3.22 (m, 1H), 3.39 (s, 3H), 3.42 (m, 2H), 4.27 (t, 2H), 5.45 (s, 1H), 5.95 (s, 2H), 6.68 (m, 2H), 6.78 (d, 1H), 6.85 (d, 1H), 7.62 (d, 1H), 8.40 (bs, 1H). LC-MS (MH⁺)=465. Anal. Cald. for C₂₅H₂₈N₄O₅ 2.9TFA 0.25H₂O: C 46.26H 3.96 N 7.01. Found: C 46.25H 3.75 N 7.20.

### Example 2

### 3-[2-(4-chlorophenyl)-1,3-thiazol-5-yl]-4-{1-methyl-5-[2-(5,6,7,8-tetrahydro-1,8-naphthyridin-2-yl)ethoxy]-1H-pyrazol-3-yl}butanoic acid.

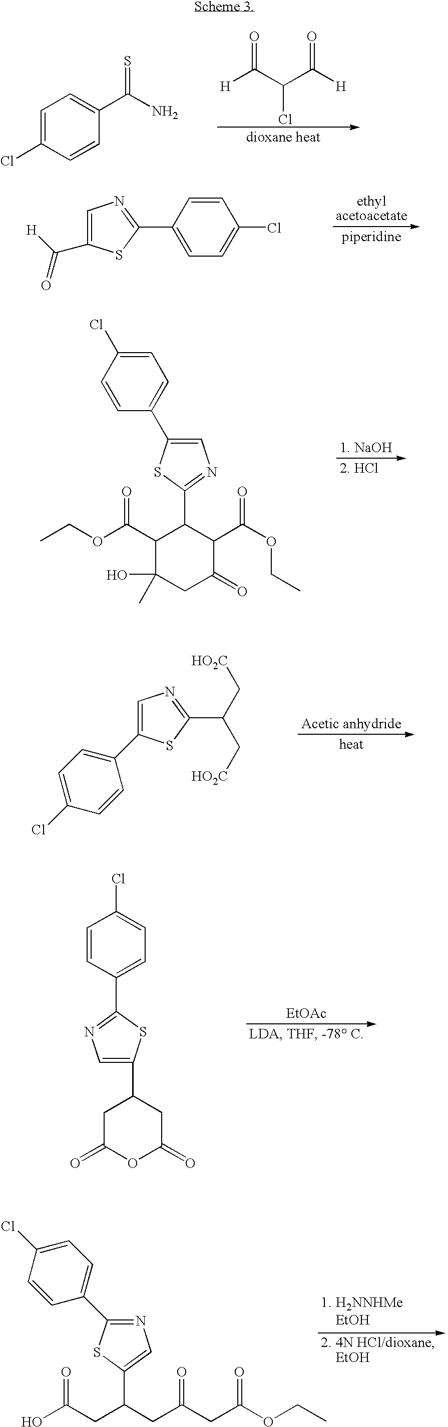

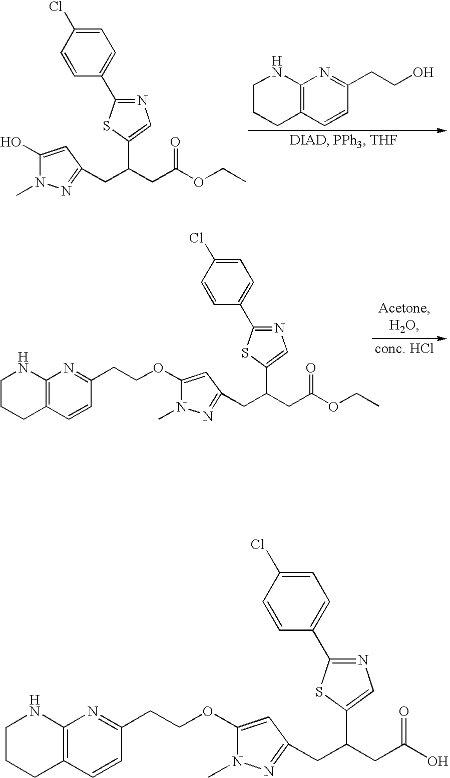

Step 1. Synthesis of 2-(4-Chlorophenyl)-1,3-thiazole-5-carbaldehyde.

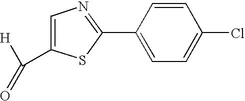

4-Chlorobenzene-1-carbothioamide (5 g, 29.1 mmol), magnesium carbonate hydroxide pentahydrate (7.06 g, 14.55 mmol), and 2-chloromalonaldehyde (4.65 g, 43.65 mmol) (Cornforth, Fawaz, Goldsworthy and Robinson; J. Chem. Soc. 1949, 1550) were added to a flask and allowed to stir under nitrogen at 60° C. for three hours. The reaction mixture was then passed through a plug of silica and washed with ethyl acetate. The solvent was removed under vacuum to give the product 2-(4-chlorophenyl)-1,3-thiazole-5-carbaldehyde (6 g, 92%) ¹H NMR (400 MHz) CDCl₃δ 10.06 (s, 1H), 8.43 (s, 1H), 7.99-7.96 (m, 2H), 7.49-7.46 (2H).

Step 2. Synthesis of 4-[2-(4-Chlorophenyl)-1,3-thiazol-5-yl]dihydro-2H-pyran-2,6(3H)-dione

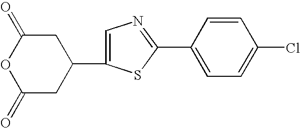

The title compound was prepared according to the general procedure outlined for 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (Example 16, WO 0196334 A2, Steps 1-3).

Step 3. Synthesis of 3-[2-(4-chlorophenyl)-1,3-thiazol-5-yl]-7-ethoxy-5,7-dioxoheptanoic acid.

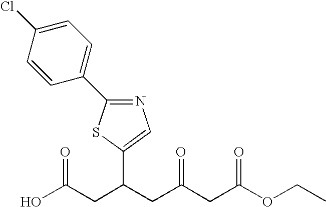

The title compound was prepared from the product of Step 2 using the procedure described in Example 1, Step 5. Compound was purified by supercritical fluid chromatography using the cyano column. ¹H NMR (400 MHz, DMSO-d6) δ 1.15 (t, 3H), 2.56-2.76 (m, 2H), 3.08 (d, 2H), 3.60 (s, 2H), 3.86 (m, 1H), 4.05 (q, 2H), 7.54 (d, 2H), 7.70 (s, 1H), 7.89 (d, 2H), 12.35 (bs, 1H). LC-MS (MH⁺)=397.

Step 4. Synthesis of ethyl 3-[2-(4-chlorophenyl)-1,3-thiazol-5-yl]-4-(5-hydroxy-1-methyl-1H-pyrazol-3-yl)butanoate.

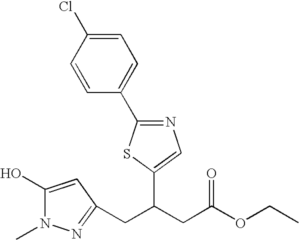

To the product of Step 3 (1.0 g, 2.5 mmol) in absolute ethanol (14 mL) at 40° C. was added methylhydrazine (148 μL, 2.8 mmol). The reaction mixture was refluxed for 2h and cooled to room temperature. To the reaction mixture was added 4N HCl in dioxane (10 mL) and the reaction stirred overnight. The reaction mixture was concentrated and partitioned between EtOAc (30 mL) and saturated aq. NaHCO₃ (30 mL). The organic layer was removed and the aqueous extracted with EtOAc (2×20 mL). The organic layers were combined, washed with brine, dried over Na₂SO₄, and concentrated to a yellow oil. The oil was purified by flash column chromatography using 5% MeOH/EtOAc as the eluent. Obtained was the desired product as yellow oil (470 mg, 1.2 mmol, 46%). ¹H NMR (400 MHz, DMSO-d6) δ 1.10 (t, 3H), 2.58-2.84 (m, 4H), 3.41 (s, 3H), 3.71 (m, 1H), 4.01 (dq, 2H), 5.12 (s, 1H), 7.55 (d, 2H), 7.65 (s, 1H), 7.88 (d, 2H), 10.69 (bs, 1H). LC-MS (MH+) 406.

Step 5. Synthesis of ethyl 3-[2-(4-chlorophenyl)-1,3-thiazol-5-yl]-4-{1-methyl-5-[2-(5,6,7,8-tetrahydro-1,8-naphthyridin-2-yl)ethoxy]-1H-pyrazol-3-yl}butanoate.

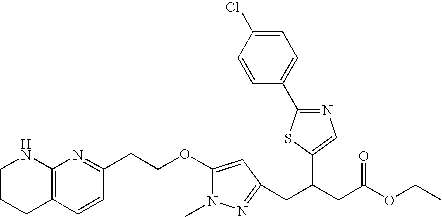

To a solution of the product of Step 4 (270 mg, 0.67 mmol) in anhydrous THF (6 mL) under Ar gas was added 2-(5,6,7,8-tetrahydro-1,8-naphthyridin-2-yl)ethanol (130 mg, 0.73 mmol) (Example 1, Step 4). When a solution formed, triphenylphosphine (210 mg, 0.80 mmol) was added. The temperature of the resulting solution was lowered to 0° C. and diisopropyl azodicarboxylate (159 μL, 0.80 mmol) was added. The reaction mixture was allowed to warm up to room temperature and stirred for 1 h. The reaction was quenched into saturated aq. NH₄Cl (15 mL) and extracted with EtOAc (3×15 mL). The organic layers were combined, washed with brine, dried over Na₂SO₄, and concentrated to an oil. The oil was purified by flash column chromatography using 5% MeOH/EtOAc as the eluent. Obtained was the desired product (226 mg). ¹H NMR (400 MHz, DMSO-d6) δ 1.10 (t, 3H), 1.75 (m, 2H), 2.60 (t, 2H), 2.62-2.87 (m, 5H), 3.23 (m, 2H), 3.38 (s, 3H), 3.75 (m, 1H), 3.99 (q, 2H), 4.23 (t, 2H), 5.48 (s, 1H), 6.30 (m, 3H), 7.03 (d, 1H), 7.53 (d, 2H), 7.66 (s, 1H), 7.87 (d, 2H). LC-MS (MH+) 567.

Step 6. Synthesis of 3-[2-(4-chlorophenyl)-1,3-thiazol-5-yl]-4-{1-methyl-5-[2-(5,6,7,8-tetrahydro-1,8-naphthyridin-2-yl)ethoxy]-1H-pyrazol-3-yl}butanoic acid.

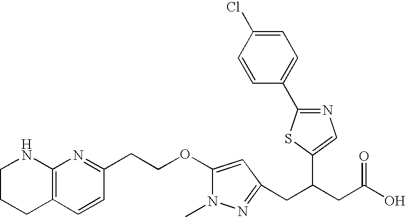

To the product of Step 5 (216 mg, 0.38 mmol) in acetone (3 mL) was added H₂O (0.2 mL) and conc. HCl (0.2 mL). The resulting reaction mixture was refluxed for 5h. The reaction mixture was cooled to room temperature and diluted with H₂O (5 mL). The acetone was removed under reduced pressure and the resulting aqueous purified by reverse phase HPLC using (H₂O/HCl)/CH₃CN as eluent (0.5 mL conc. HCl in 4 L H₂O). Obtained was the HCl salt desired product as yellowish solid (135 mg). ¹H NMR (400 MHz, DMSO-d6) δ 1.81 (m, 2H), 2.55-2.93 (m, 6H), 3.11 (t, 2H), 3.44 (m, 5H), 3.76 (m, 1H), 4.37 (t, 2H), 5.70 (s, 1H), 6.67 (d, 1H), 7.53 (d, 2H), 7.59 (d, 1H), 7.67 (s, 1H), 7.87 (d, 2H), 8.15 (bs, 1H). LC-MS (MH⁺) 539. Anal. Cald. for C₂₇H₂₈ClN₅O₃S 2.8HCl 3H₂O: C 46.71H 5.34 N 10.09. Found: C 46.59H 5.46 N 10.07.

### Example 3

### 3-(1,3-benzodioxol-5-yl)-4-(1-methyl-5-{[2-(5,6,7,8-tetrahydro-1,8-naphthyridin-2-yl)ethyl]thio}-1H-pyrazol-3-yl)butanoic acid

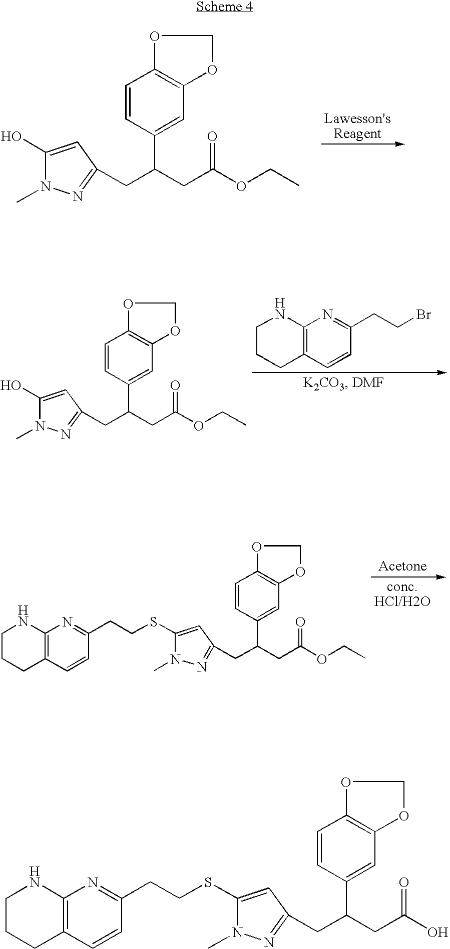

Step 1. Synthesis of ethyl 3-(1,3-benzodioxol-5-yl)-4-(5-mercapto-1-methyl-1H-pyrazol-3-yl)butanoate.

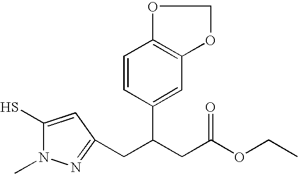

To a solution of ethyl 3-(1,3-benzodioxol-5-yl)-4-(5-hydroxy-1-methyl-1H-pyrazol-3-yl)butanoate (460 mg, 1.4 mmol) in benzene (10 mL) was added Lawesson's reagent (336 mg, 0.83 mmol). The resulting mixture was heated at 60° C. overnight. The resulting solution was concentrated to an oil and purified by flash column chromatography using 50% EtOAc/ Hexane, followed by 5% MeOH/EtOAc as eluents. Obtained was the desired product as a yellow oil (236 mg). ¹H NMR (400 MHz, DMSO-d6) δ 1.06 (t, 3H), 2.51-2.69 (m, 2H), 2.79 (d, 2H), 3.30 (m, 2H), 3.56 (bs, 3H), 3.95 (dq, 2H), 5.67 (bs, 1H), 5.97 (s, 2H), 6.67 (dd, 1H), 6.78 (d, 1H), 6.89 (d, 1H). LC-MS (MH⁺) 349.

Step 2. Synthesis of ethyl 3-(1,3-benzodioxol-5-yl)-4-(1-methyl-5-{[2-(5,6,7,8-tetrahydro-1,8-naphthyridin-2-yl)ethyl]thio}-1H-pyrazol-3-yl)butanoate

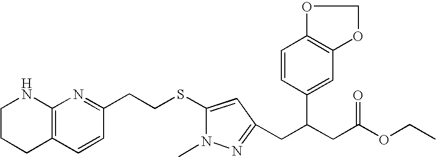

To a mixture of the product of Step 1 (200 mg, 0.57 mmol) and K₂CO₃ (87 mg, 0.63 mmol) in anhydrous DMF (10 mL) under Ar gas at 60° C. was added a solution of 7-(2-bromoethyl)-1,2,3,4-tetrahydro-1,8-naphthyridine (152 mg, 0.63 mmol) (Example 9, Step 1) in anhydrous DMF (2.5 mL). The reaction mixture was stirred for 4 h at 60° C. The reaction mixture was quenched into water (25 mL) and extracted with EtOAc (3×20 mL). The organic layers were combined, washed with brine, dried over Na₂SO₄, and concentrated to an oil. The oil was purified by flash column chromatography using 2.5% MeOH/EtOAc as the eluent. Obtained was the desired product as a reddish oil (252 mg). ¹H NMR (400 MHz, DMSO-d6) δ 1.04 (t, 3H), 1.74 (m, 2H), 2.51-2.68 (m, 6H), 2.76 (m, 2H), 2.99 (t, 2H), 3.23 (m, 2H), 3.26 (m, 1H), 3.69 (s, 3H), 3.92 (dq, 2H), 5.93 (d, 2H), 6.04 (s, 1H), 6.21 (d, 1H), 6.30 (m, 1H), 6.67 (dd, 1H), 6.75 (s, 1H), 6.87 (d, 1H), 7.03 (d, 1H). LC-MS (MH⁺) 509.

Step 3. Synthesis of 3-(1,3-benzodioxol-5-yl)-4-(1-methyl-5-{[2-(5,6,7,8-tetrahydro-1,8-naphthyridin-2-yl)ethyl]thio}-1H-pyrazol-3-yl)butanoic acid.

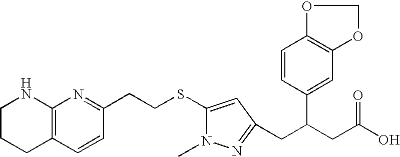

The title compound was prepared from the product of Step 2 using the procedure described in Example 2, step 6. ¹H NMR (400 MHz, DMSO-d6) δ 1.82 (m, 2H), 2.43-2.63 (m, 2H), 2.67-2.88 (m, 6H), 3.21 (t, 2H), 3.25 (m, 1H), 3.43 (m, 2H), 3.71 (s, 3H), 5.93 (d, 2H), 6.11 (s, 1H), 6.54 (d, 1H), 6.67 (dd, 1H), 6.75 (d, 1H), 6.86 (d, 1H), 7.58 (d, 1H), 8.17 (bs, 1H). MS (ESI+) for C₂₅H₂₈N₄O₄S m/z 481.1926 (M+H)⁺. Anal. Cald. for C₂₅H₂₈N₄O₄S 2.25HCL 1.75H₂O: C 50.54H 5.73 N 9.43 S 5.40. Found: C 50.37H 5.99 N 9.49 S 5.58.

### Example 4

### Synthesis of 3-(1,3-benzodioxol-5-yl)-4-(1-methyl-5-{[2-(5,6,7,8-tetrahydro-1,8-naphthyridin-2-yl)ethyl]sulfonyl}-1H-pyrazol-3-yl)butanoic acid

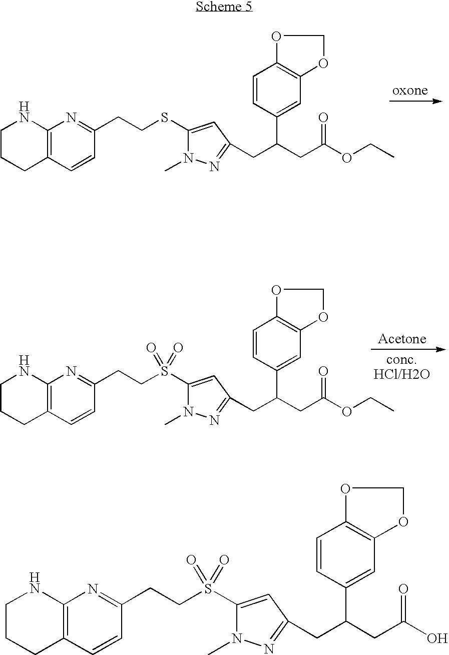

Step 1. Synthesis of ethyl 3-(1,3-benzodioxol-5-yl)-4-(1-methyl-5-{[2-(5,6,7,8-tetrahydro-1,8-naphthyridin-2-yl)ethyl]sulfonyl}-1H-pyrazol-3-yl)butanoate.

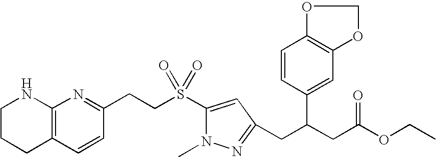

A mixture of oxone (480 mg, 0.78 mmol) in water (1.8 mL) and MeOH (1.2 mL) was added drop wise to a solution of the product of Example 3, Step 2 (200 mg, 0.39 mmol) in THF (2.5 mL) at room temp. The reaction mixture was stirred at room temp for 2 h and poured into water (10 mL). The mixture was extracted with EtOAc (3×15 mL). The organic layers were combined, washed with brine, dried over Na₂SO₄, and concentrated to the desired product (167 mg). ¹H NMR (400 MHz, DMSO-d6) δ 1.04 (t, 3H), 1.80 (m, 2H), 2.51-2.69 (m, 2H), 2.71 (t, 2H), 2.83 (m, 2H), 2.96 (t, 2H), 3.29 (m, 1H), 3.40 (m, 2H), 3.91 (m, 4H), 3.99 (s, 3H), 5.93 (dd, 2H), 6.52 (s, 1H), 6.63 (d, 1H), 6.67 (dd, 1H), 6.74 (d, 1H), 6.89 (d, 1H), 7.56 (d, 1H). LC-MS (MH⁺) 541.

Step 2. Synthesis of 3-(1,3-benzodioxol-5-yl)-4-(1-methyl-5-{[2-(5,6,7,8-tetrahydro-1,8-naphthyridin-2-yl)ethyl]sulfonyl}-1H-pyrazol-3-yl)butanoic acid

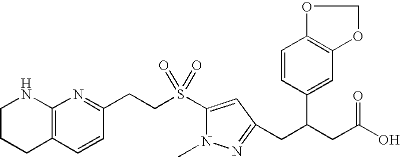

The title compound was prepared from the product of Step 1 using the procedure described in Example 2, step 6. ¹H NMR (400 MHz, DMSO-d6) δ 1.80 (m, 2H), 2.43-2.63 (m, 2H), 2.72 (t, 2H), 2.83 (m, 2H), 2.97 (t, 2H), 3.27 (m, 1H), 3.42 (m, 2H), 3.93 (t, 2H), 3.99 (s, 3H), 5.92 (d, 2H), 6.48 (s, 1H), 6.65 (dd, 2H), 6.75 (d, 1H), 6.87 (d, 1H), 7.58 (d, 1H), 7.98 (bs, 1H). Anal. Cald. for C₂₅H₂₈N₄O₆S 1.8HCl 2H₂O: C 48.89H 5.55 N 9.12 S 5.22. Found: C 48.79H 5.69 N 9.10 S 5.45.

### Example 5

### (3S)-3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoic acid

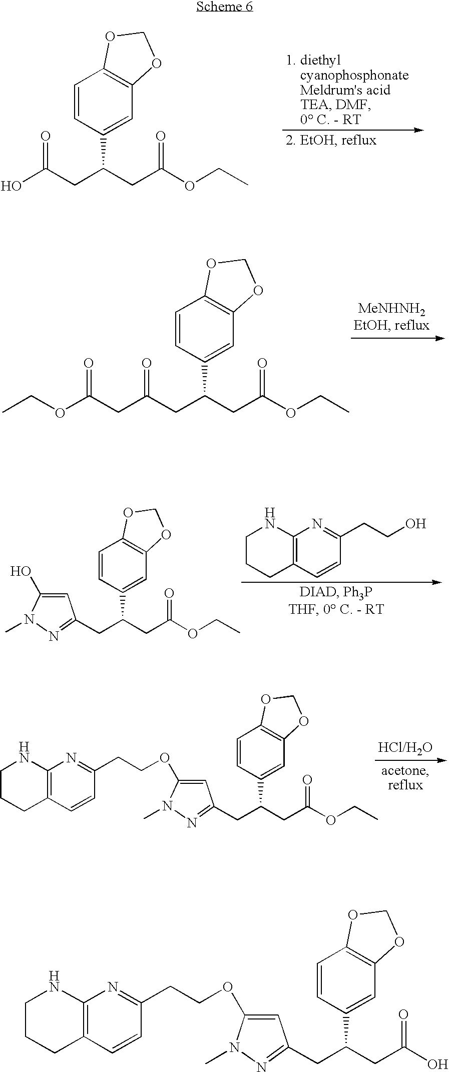

Step 1. Synthesis of diethyl (3S)-3-(1,3-benzodioxol-5-yl)-5-oxoheptanedioate.

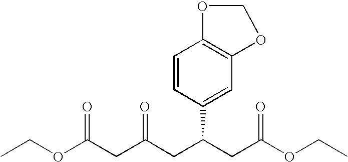

To (3S)-3-(1,3-benzodioxol-5-yl)-5-ethoxy-5-oxopentanoic acid (30 g, 0.11 mol) (prepared via chiral chromatographic resolution of the EtOH opening product of the anhydride described in WO0196334 A2) and Meldrum's acid (17.4 g, 0.12 mol) in anhydrous DMF (230 mL) under Ar gas at 0° C. was slowly added diethyl cyanophosphonate (19.5 mL, 0.13 mol), followed by Et₃N (48 mL, 0.34 mol). The reaction mixture was stirred at 0° C. for 30 min. and at room temp overnight. The reaction was quenched into ice cold 2N HCl (250 mL) and stirred for 5 min. The mixture was diluted with water (250 mL) and extracted with EtOAc (3×250 mL) The organic layers were combined, washed with water, washed with brine, dried over Na₂SO₄, and concentrated to an oil. The oil was redissolved in absolute EtOH (750 mL) and refluxed for 3 h. The reaction mixture was concentrated to an oil and purified by flash column chromatography using 20% EtOAc/hexane as the eluent. Obtained was the desired product as an oil (27.5 g). ¹H NMR (400 MHz, CDCl₃) δ 1.17 (t, 3H), 1.24 (t, 3H), 2.50-2.68 (m, 2H), 2.90 (m, 2H), 3.34 (s, 2H), 3.63 (m, 1H), 4.04 (dq, 2H), 4.15 (q, 2H), 5.92 (s, 2H), 6.69 (m, 3H).

Step 2. Synthesis of ethyl (3S)-3-(1,3-benzodioxol-5-yl)-4-(5-hydroxy-1-methyl-1H-pyrazol-3-yl)butanoate

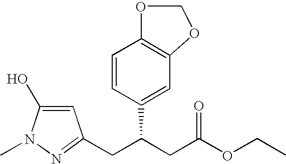

To a solution of the product of Step 1 (27.4 g, 78.2 mmol) in absolute EtOH (400 mL) at room temp was added methylhydrazine (4.6 mL, 86 mmol). The reaction mixture was refluxed for 1.5 h and concentrated to the desired product (25.8 g). ¹H NMR (400 MHz, DMSO-d6) δ 1.04 (t, 3H), 2.47 (m, 1H), 2.61 (m, 3H), 3.20 (m, 1H), 3.37 (s, 3H), 3.91 (dq, 2H), 4.98 (s, 1H), 5.95 (s, 2H), 6.65 (dd, 1H), 6.77 (d, 1H), 6.85 (d, 1H).

Step 3. Synthesis of ethyl (3S)-3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoate.

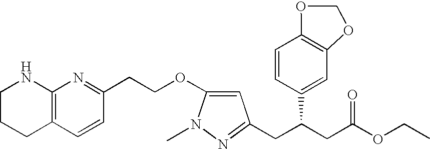

To a solution of triphenylphosphine (24.4 g, 93 mmol) in anhydrous THF (322 mL) at −10° C. was drop wise added diisopropyl azodicarboxylate (18.3 mL, 93 mmol). The reaction mixture was stirred at −10° C. for 20 min. To the reaction mixture was drop wise added a solution of 2-(5,6,7,8-tetrahydro-1,8-naphthyridin-2-yl)ethanol ((15.2 mL, 85 mmol) in anhydrous THF (35 mL). The reaction mixture was stirred at −10° C. for 20 min and a solution of the product of Step 2 (25.7 g, 77 mmol) in anhydrous THF (80 mL) was added in one portion. The reaction mixture was allowed to warm up to room temp and stirred for 5 h. The reaction mixture was quenched into saturated aqueous NH₄Cl (300 mL) and extracted with EtOAc (3×300 mL). The organic layers were combined, washed with brine, dried over Na₂SO₄, and concentrated under reduced pressure. The residual oil was purified by flash column chromatography using 70% EtOAc/hexane, followed by 5% MeOH/EtOAc as eluents. Obtained was the desired product as an oil (14.4 g). ¹H NMR (400 MHz, DMSO-d6) δ 1.04 (t, 3H), 1.75 (m, 2H), 2.5 (m, 1H), 2.63 (m, 5H), 2.84 (t, 2H), 3.24 (m, 3H), 3.36 (s, 3H), 3.91 (dq, 2H), 4.22 (t, 2H), 5.38 (s, 1H), 5.95 (s, 2H), 6.33 (m, 2H), 6.67 (dd, 1H), 6.77 (d, 1H), 6.86 (d, 1H), 7.05 (d, 1H). LC-MS (MH⁺)=493.

Step 4. Synthesis of (3S)-3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoic acid.

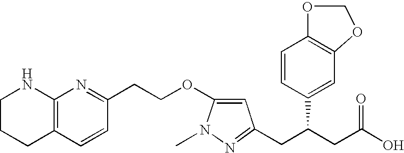

The title compound was prepared from the product of Step 3 using the procedure described in Example 2, step 6. ¹H NMR (400 MHz, DMSO-d6) δ 1.81 (m, 2H), 2.38-2.61 (m, 2H), 2.64 (m, 2H), 2.73 (t, 2H), 3.10 (t, 2H), 3.23 (m, 1H), 3.39 (s, 3H), 3.42 (m, 2H), 4.30 (t, 2H), 5.44 (s, 1H), 5.95 (s, 2H), 6.65 (m, 2H), 6.77 (d, 1H), 6.84 (d, 1H), 7.59 (d, 1H), 8.04 (bs, 1H), 11.97 (bs, 1H). MS (ESI+) for C₂₅H₂₈N₄O₅ m/z 465.2155 (M+H)⁺. Anal. Cald. for C₂₅H₂₈N₄O₅ HCl H₂O: C 57.86H 6.02 N 10.80. Found: C 57.82H 6.22 N 10.82.

### Example 6

### 3-(2-cyclopropyl-1,3-thiazol-5-yl)-4-{1-methyl-5-[2-(5,6,7,8-tetrahydro-1,8-naphthyridin-2-yl)ethoxy]-1H-pyrazol-3-yl}butanoic acid.

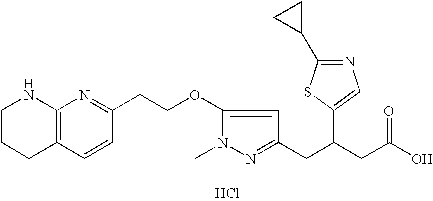

3-(2-cyclopropyl-1,3-thiazol-5-yl)-4-{1-methyl-5-[2-(5,6,7,8-tetrahydro-1,8-naphthyridin-2-yl)ethoxy]-1H-pyrazol-3-yl}butanoic acid hydrochloride was made according to the procedure for making 3-[2-(4-chlorophenyl)-1,3-thiazol-5-yl]-4-{1-methyl-5-[2-(5,6,7,8-tetrahydro-1,8-naphthyridin-2-yl)ethoxy]-1H-pyrazol-3-yl}butanoic acid (Example 2) using cyclopropylcarbothioamide in place of 4-chlorobenzene-1-carbothioamide. ¹H NMR (400 MHz, DMSO-d6) δ 7.95 (br s, 1H), 7.6 (d, 1H), 7.28 (s, 1H), 6.67 (d, 1H), 5.5 (s, 1H), 4.3 (t, 2H), 3.58 (m, 1H), 3.45 (m, 2H), 3.4 (s, 3H), 3.1 (t, 2H), 2.73-2.62 (m, 4H), 2.45 (m, 2H), 2.25 (m, 1H), 1.81 (m, 2H), 1.05 (m, 2H), 0.85 (m, 2H); Mass Spectrum: (MH⁺)=468.20. +)=469.1.

### Example 7

### 3-(6-methoxypyridin-3-yl)-4-{1-methyl-5-[2-(1-methyl-1,2,3,4-tetrahydropyrido[2,3-b]pyrazin-6-yl)ethoxy]-1H-pyrazol-3-yl}butanoic acid

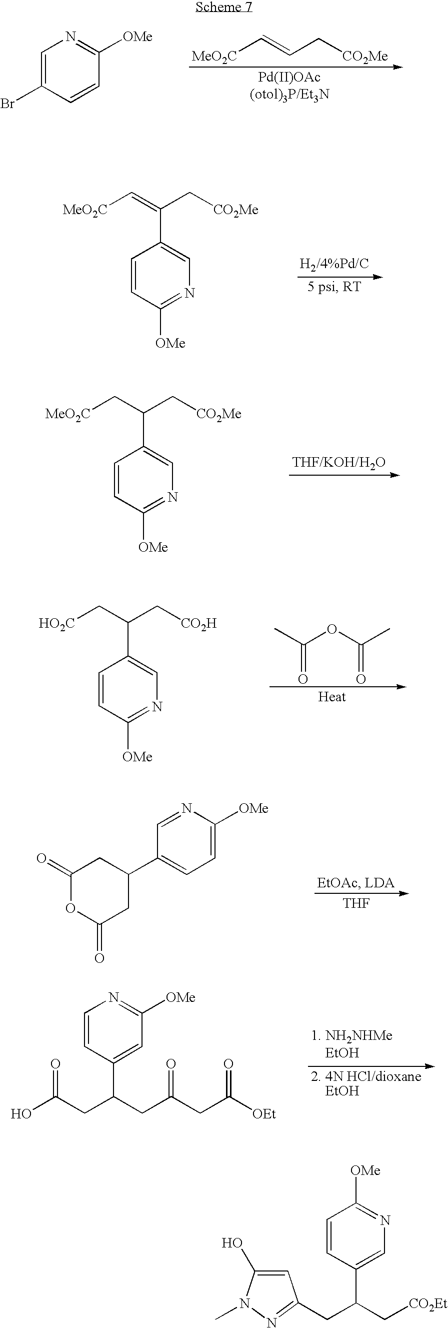

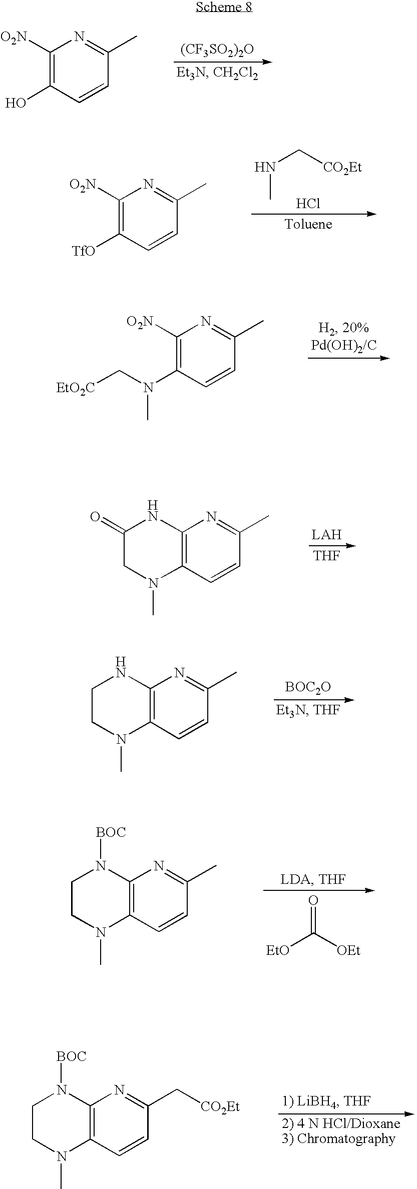

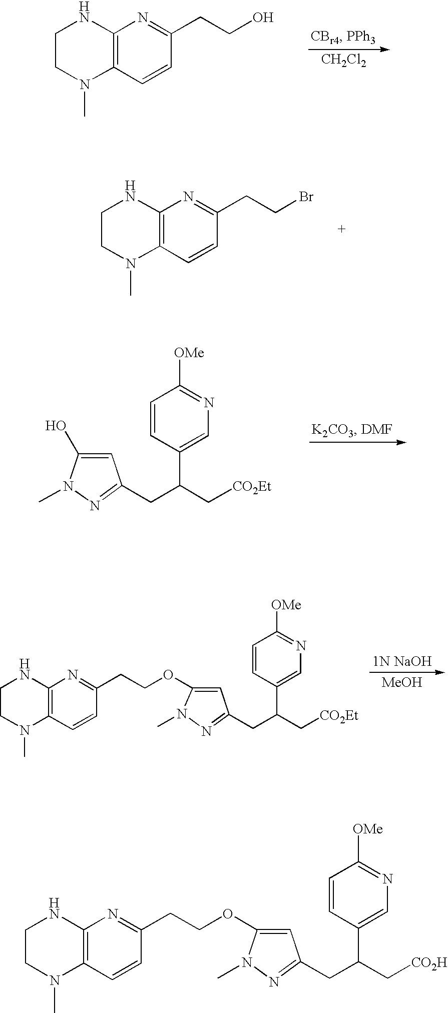

Step 1. Synthesis of dimethyl 3-(6-methoxypyridin-3-yl)pent-2-enedicarboxylate.

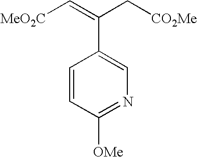

A mixture of dimethyl pent-2-enedicarboxylate (2.86 g, 18.09 mmol), Palladium (II) acetate (0.12 g, 0.53 mmol), tri-o-tolyphosphine (0.405 g, 1.33 mmol), and triethylamine (2.0 mL) in DMF (2.13 mL) was degassed and heated at 90° C. 5-Bromo-2-methoxy pyridine was added drop wise to the mixture and heated at 90° C. overnight. The reaction mixture was cooled to rt and the solid was filtered. The filtrate was diluted with water and this mixture was extracted with ethyl acetate (3×100 mL). The organic layers were combined, washed with brine, dried over Na₂SO₄ and concentrated under reduced pressure. The resulting residue was purified by flash column chromatography using 5-25% EtOAc/Hexane to give the product as a light yellow oil (0.301 g, 21%). ¹H NMR (CD₃OD) δ 8.31 (d, 1H), 7.88-7.84 (m, 1H), 6.84 (d, 1H), 6.33 (s, 1H), 4.86 (s, 2H), 3.95 (s, 3H), 3.75 (s, 3H), 3.68 (s, 3H);
Step 2. Synthesis of dimethyl 3-(6-methoxypyridin-3-yl)pentanedicarboxylate.

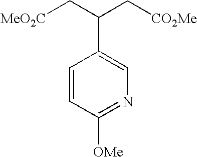

A standard par bottle was charged with dimethyl 3-(6-methoxypyridin-3-yl)pent-2-enedicarboxylate (0.301 g, 1.13 mmol) in MeOH and 4% Palladium on carbon. The hydrogenation was carried out at 5 psi at rt for two hours to give the title compound. MS (ESI+) for C₁₃H₁₇NO₅ m/z 268.40 (M+H)⁺.

Step 3. Synthesis of 3-(6-methoxypyridin-3-yl)pentanedioic acid.

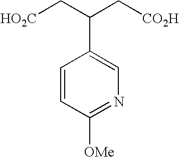

To the product of Step 2 (0.276 g, 1.034 mmol) in THF (17.20 mL) was added water (17.20 mL) and KOH (0.58 g). The reaction mixture was stirred at rt for overnight. Concentrated HCl was then added until the pH=2.0. During the addition, the temperature was kept below 50 C. The mixture was extracted with ethyl acetate (3×50 mL). The organic layers were combined, washed with brine, dried over Na₂SO₄ and concentrated to produce an off white solid (0.145 g, 59%). ¹H NMR (CD₃OD) δ 8.05 (d, 1H), 7.69-7.65 (m, 1H), 6.78 (d, 1H), 3.89 (s, 3H), 3.60-3.51 (m, 1H), 2.80-2.73 (m, 2H), 2.65-2.58 (m, 2H); MS (ESI+) for C₁₁H₁₃NO₅ m/z 240.30 (M+H)⁺.

Step 4. Synthesis of 4-(6-methoxypyridin-3-yl)dihydropyran-2,6(3H)-dione.

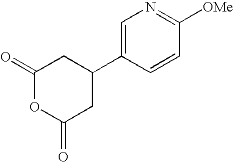

To the product of step 3 (0.276 g, 1.15 mmol) was added acetic anhydride (10.0 mL). The reaction mixture was stirred and heated at 100 C for 5 hours. The reaction mixture was cooled to rt. The solvent was removed under reduced pressure to give a dark brown solid (0.086 g, 34%). LCMS was done by diluting the sample with acetonitrile and adding 50 uL of Piperidine, LCMS indicated mass product 307.40 m/z (M+Piperidine).

Step 5. Synthesis of 7-ethoxy-3-(6-methoxypyridin-3-yl)-5,7-dioxoheptanoic acid.

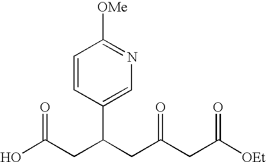

To a solution of anhydrous EtOAc (9.27 mL, 94.9 mmol) in anhydrous THF (37 mL) at −78° C. under Ar gas was slowly added lithium diisopropylamide (2M in heptane/THF/ethylbenzene, 47.5 mL, 94.9 mmol). The resulting solution was stirred at −78° C. for 25 min and added drop wise via cannula to a solution of the product of Step 4 (10 g, 45.2 mmol) in anhydrous THF (250 mL) at −78° C. under Ar gas. The reaction mixture was stirred at −78° C. for 1.5 h. The reaction mixture was quenched with 2N HCl in ether (100 mL) and allowed to warm up to room temperature. To the reaction mixture was added water (200 mL) and extracted with EtOAc (2×100 mL). The aqueous layer was basified to PH=4 with 2N NaOH solution and extracted with EtOAc (3×150 mL). The organic layers were combined, washed with brine, dried over Na₂SO₄, and concentrated under reduced pressure. The resulting residue was purified by flash column chromatography using 80% EtOAc/hexane to give 2.28 g of the title compound as a brown oil. ¹H NMR (400 MHz, CDCl₃) δ 1.24 (t, 3H), 2.57-2.81 (m, 4H), 2.86-3.03 (m, 2H), 3.66 (m, 1H), 4.15 (m, 2H), 6.69 (d, 1H), 7.46 (dd, 1H), 8.05 (d, 1H).

Step 6. Synthesis of ethyl 4-(5-hydroxy-1-methyl-1H-pyrazol-3-yl)-3-(6-methoxypyridin-3-yl)butanoate.

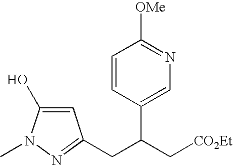

Methyl hydrazine (432 μL, 8.12 mmol) was added drop wise to a stirred solution of the product of step 5 (2.28 g, 7.38 mmol) in absolute ethanol (60 mL) at room temperature. After complete addition, the reaction mixture was refluxed for 3 h. The solvent was removed under reduced pressure. The resulting residue was dissolved in absolute ethanol (10 mL) and 4N HCl in dioxane (15 mL) was added. The reaction mixture was stirred at room temperature overnight. The solvent was removed under reduced pressure and the residue purified by flash column chromatography using 3% MeOH/EtOAc as eluent to obtain a yellow oil (0.8 g, 34%). ¹H NMR (400 MHz, CDCl₃) δ1.17 (t, 3H), 2.55-2.83 (m, 4H), 3.23 (s, 3H), 3.45 (m, 1H), 3.92 (s, 3H), 4.85 (q, 2H), 5.31 (s, 1H), 6.71 (d, 1H), 7.94 (dd, 1H), 8.01 (d, 1H).

Step 7. Synthesis of 6-methyl-2-nitropyridin-3-yl trifluoromethanesulfonate.

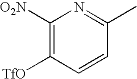

To a solution of 3-hydroxy-6-methyl-2-nitropyridine (2 g, 12.97 mmol) in CH₂Cl₂ (150 mL) at 0° C. under N₂ was added triethylamine (2.68 mL, 19.27 mmol) and followed by trifluoromethanesulfonic anhydride (2.62 mL, 15.57 mmol). The mixture was stirred for 2 hours at 0° C. and then quenched with water. The organic layer was separated, washed with water and dried over MgSO4. After filtration and concentration at reduced pressure, the crude mixture was purified by flash chromatography on silica gel (15% EA/Hex) to afford the desired product (3.65 g, 98%) as a yellow oil. ¹H NMR (400 MHz, CDCl₃) δ 2.70 (s, 3H), 7.59 (d, 1H), 7.81(d, 2H).

Step 8. Synthesis of ethyl N-methyl-N-(6-methyl-2-nitropyridin-3-yl)glycinate.

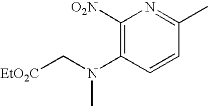

To a solution of the product of step 7 (7 g, 24.47 mmol) in toluene (40 mL) at room temperature under N₂ was added sarcosine ester hydrochloride (9.4 g, 61.2 mmol) and followed by triethylamine (8.51 mL, 61.2 mmol). The mixture was refluxed overnight under N₂. The reaction was cooled to room temperature and quenched with water. The mixture was extracted three times with ethyl acetate and all organic extracts were combined, washed with brine, dried over Na₂SO₄. After filtration and concentration at reduced pressure, the crude mixture was purified by flash chromatography on silica gel (20% EA/Hex) to afford the desired product (4.3 g, 69%) as brown oil. ¹H NMR (400 MHz, CDCl₃) δ 1.026 (t, 3H), 2.50 (s, 3H), 2.95 (s, 3H), 3.88 (s, 2H), 4.20 (q, 2H), 7.27 (d, 1H), 7.49(d, 2H).

Step 9. Synthesis of 1,6-dimethyl-1,4-dihydropyrido[2,3-b]pyrazin-3(2H)-one.

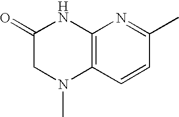

The product of step 8 (4.3 g, 17 mmol) was hydrogenated in ethanol solution at room temperature using H₂ at 5 psi and 20% Pd(OH)₂/C catalyst for 2 h. Upon completion of the reaction, the catalyst was filtered off and the filtrate was concentrated under reduced pressure. The product was crystallized out from 50% EA/Hex solution as yellow crystalline solid. The mother liquid was concentrated and purified by flash chromatography on silica gel (50% EA/Hex). (1.44 g, 46%) H NMR (400 MHz, CDCl₃) δ 2.26 (s, 3H), 2.70 (s, 3H), 3.18 (t, 2H), 3.58 (m, 2H), 6.34 (d, 1H), 6.57(d, 2H). Step 10. Synthesis of 1,6-dimethyl-1,2,3,4-tetrahydropyrido[2,3-b]pyrazine.

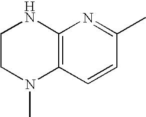

LiAlH₄ (214 mg, 5.64 mmol) was slowly added to 10 mL anhydrous THF in a round-bottom flask fitted with a stir bar and a condenser. After stirring for 10 minutes, a solution of the product of step 9 (500 mg, 2.82 mmol) in 5 mL anhydrous THF was added drop wise. Upon completion of the addition, the reaction mixture was refluxed for 16 hours. The reaction was cooled to room temperature and quenched with 1M NaOH solution until the mixture had become a milky yellow color. The precipitate was filtered off and washed 3 times with CH₂Cl₂. The filtrate and washings were combined, washed with brine, dried over MgSO₄. Filtered and concentrated under reduced pressure to give the desired product as light yellow oil, which solidified on standing. (420 mg, 91%). ¹H NMR (400 MHz, CDCl₃) δ2.27 (s, 3H), 2.80 (s, 3H), 3.17 (t, 2H), 3.58 (m, 2H), 6.36 (d, 1H), 6.56(d, 2H).

Step 11. Synthesis of tert-butyl 1,6-dimethyl-2,3-dihydropyrido[2,3-b]pyrazine-4(1H)-carboxylate.

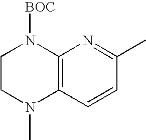

A solution of the product of step 10 (1.14 g, 7 mmol), di-tert-butyl dicarbonate (2.29 g, 10.5 mmol), DMAP (100 mg) and triethylamine (1.46 mL, 10.5 mmol) in 30 mL THF was refluxed 72 hours under N₂. The reaction mixture was allowed to cool to room temperature and diluted with ethyl acetate. The mixture was washed with brine, dried over Na₂SO₄. After filtration and concentration at reduced pressure, the crude mixture was purified by flash chromatography on silica gel (40% EA/Hex) to afford the desired product (1.6 g, 90%) as yellow oil. ¹H NMR (400 MHz, CDCl₃) δ 1.51 (s, 9H), 2.40 (s, 3H), 2.90 (s, 3H), 3.28 (t, 2H), 3.83 (m, 2H), 6.78 (d, 1H), 6.83(d, 2H).

Step 12. Synthesis of tert-butyl 6-(2-ethoxy-2-oxoethyl)-1-methyl-2,3-dihydropyrido[2,3-b]pyrazine-4(1H)-carboxylate.

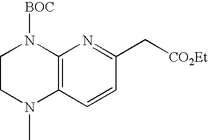

Lithium diisopropylamide solution (5 mL, 10 mmol, 2.0 M in THF/ethylbenzene/heptane) was added drop wise to a chilled (−78° C.), stirred solution of the product of step 11 (950 mg, 3.61 mmol) and diethyl carbonate (1.62 mL, 13.36 mmol) in 20 mL dry THF under nitrogen atmosphere. After 1 hour the reaction was quenched with saturated NH₄Cl solution and warmed to room temperature. The mixture was extracted three times with ethyl acetate and all organic extracts were combined, dried over Na₂SO₄, and concentrated under reduced pressure to get the crude product, which was purified by chromatography on silica gel (30% ethyl acetate/hexane). The desired fractions were combined and concentrated under reduced pressure to get the desired product (1.05 g, 87%) as a yellow solid. ¹H NMR (400 MHz, CDCl₃) δ 1.25 (t, 3H), 1.50 (s, 9H), 2.78 (s, 3H), 3.38 (t, 2H), 3.68(s, 2H), 3.84 (t, 2H), 4.14 (q, 2H), 6.86 (d, 1H), 6.95(d, 2H).

Step 13. Synthesis of 2-(1-methyl-1,2,3,4-tetrahydropyrido[2,3-b]pyrazin-6-yl)ethanol.

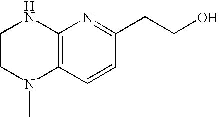

To a solution of the product of step 12 (1.05 g, 3.13 mmol)) in dry THF (15 mL) at room temperature was added a solution of LiBH₄ (2.0 M in THF, 1.88 mL), and the resulting mixture was heated to reflux. After 16 hours the mixture was cooled to 0° C. and carefully quenched with water (20 mL). After 10 minutes, the mixture was extracted three times with ethyl acetate. The combined organic extracts were dried over MgSO₄, filtered, and concentrated under reduced pressure. This residue was dissolved in CH₂Cl₂ (3 mL), and to this solution was added 4 M HCl in dioxane (6 mL) all at once at room temp. After 4 hours, the mixture was concentrated under reduced pressure to get the crude product, which was chromatographed on silica gel (eluent: 98/2/0.5 dichloromethane/methanol/-ammonium hydroxide) to afford the desired product as a gray solid. (390 mg) H1 NMR (400 MHz, CDCl₃) δ 2.73 (t, 2H), 2.72 (s, 3H), 3.20 (t, 2H), 3.58(m, 2H), 3.89 (t, 2H), 6.36 (d, 1H), 6.58(d, 2H).

Step 14. Synthesis of 6-(2-bromoethyl)-1-methyl-1,2,3,4-tetrahydropyrido[2,3-b]pyrazine.

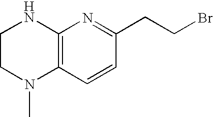

To a solution of the product of step 13 (2.1 g, 10.87 mmol) in CH₂CL₂ (60 mL) at 0° C. under N₂ was added triphenylphosphine (3.14 g, 11.96 mmol) and followed by carbon tetrabromide (3.97 g, 11.96 mmol). The ice bath was removed and the reaction mixture was concentrated under reduced pressure. The black residue was partitioned between 2N HCl solution and EA. Layers were separated and the aqueous layer was washed with EA (2×) and then basified to PH=5 with 2N NaOH. This aqueous layer was extracted with EA (3×) and washed with brine, dried over Na₂SO₄ and concentrated under reduced pressure. The residue was purified by reverse phase HPLC using (H₂O/TFA)/CH₃CN as eluent (2.5 mL TFA in 4 L H₂O) to afford 700 mg of the title compound as greenish solid. H₁NMR (400 MHz, CD₃OD) δ2.93 (s, 3H), 3.19 (t, 2H), 3.25 (t, 2H), 3.65 (t, 2H), 3.71 (t, 2H), 6.45 (d, 1H), 6.64 (d, 1H).

Step 15. Synthesis of ethyl 3-(6-methoxypyridin-3-yl)-4-{1-methyl-5-[2-(1-methyl-1,2,3,4-tetrahydropyrido[2,3-b]pyrazin-6-yl)ethoxy]-1H-pyrazol-3-yl}butanoate.

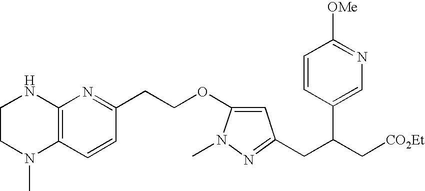

The mixture of the product of step 14 (130 mg, 0.5 mmol), DMF (5 mL), the product of step 6 (147 mg, 0.46 mmol) and K₂CO₃ (63 mg, 0.46 mmol) was heated to 60° C. overnight. The mixture was diluted with water, extracted with ethyl acetate. The ethyl acetate layer was washed with water, brine and then dried with Na₂SO₄. The solvent was removed and the residue was purified by reverse phase HPLC using (H₂O/TFA)/CH₃CN as eluent (2.5 mL TFA in 4 L H₂O) to afford 110 mg of the title compound as brown oil. ¹H NMR (400 MHz, CD₃OD) δ 1.18 (t, 3H), 2.65-2.95 (m, 4H), 3.02 (s, 3H), 3.16 (t, 2H), 3.53 (s, 3H), 3.74 (t, 2H), 3.97 (s, 3H), 4.07 (q, 2H), 4.35 (t, 2H), 5.54 (s, 1H), 6.75 (d, 1H), 6.89 (d, 1H), 7.00 (d, 1H), 7.74 (dd, 1H), 7.97 (d, 1H).

Step 16. Synthesis of 3-(6-methoxypyridin-3-yl)-4-{1-methyl-5-[2-(1-methyl-1,2,3,4-tetra hydropyrido[2,3-b]pyrazin-6-yl)ethoxy]-1H-pyrazol-3-yl}butanoic acid.

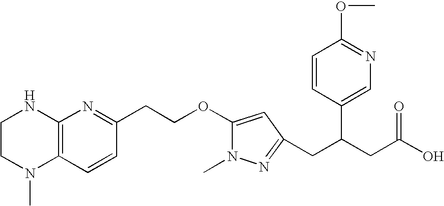

The product of step 15 (170 mg, 0.34 mmol) was dissolved in 2 ml methanol and 2 ml 1N sodium hydroxide solution. The reaction was stirred at room temperature overnight, concentrated and acidified with 1 ml trifluoroacetic acid, then purified by reverse phase HPLC using (H₂O/TFA)/CH₃CN as eluent (2.5 mL TFA in 4 L H₂O) to yield 150 mg desired product as orange color oil. FAB-MS:(MH⁺)=467. ¹H NMR (500 MHz, CD₃OD) δ 2.60-2.92 (m, 4H), 2.93 (s, 3H), 3.09 (t, 2H), 3.29 (t, 2H), 3.45 (m, 4H), 3.66 (t, 2H), 3.96 (s, 3H), 4.30 (t, 2H), 5.57(s, 1H), 6.65 (d, 1H), 6.90 (d, 1H), 7.01 (d, 1H), 7.88 (dd 1H), 8.00 (d, 1H). Anal Calcd. for C₂₄H₃₀N₆O₄ plus 3.8 CF₃COOH and 2H₂O: C, 40.56; H, 4.07; N, 8.98. Found: 40.57; H, 4.39; N, 8.90.

### Example 8

### 3-(1,3-benzodioxol-5-yl)-4-(1-methyl-5-{2-[6-(methylamino)pyridin-2-yl]ethoxy}-1H-pyrazol-3-yl)butanoic acid

Step 1. Synthesis of ethyl 3-(1,3-benzodioxol-5-yl)-4-(1-methyl-5-{2-[6-(methylamino)pyridin-2-yl]ethoxy}-1H-pyrazol-3-yl)butanoate.

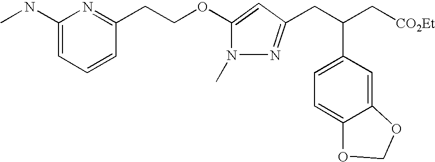

To 2-[6-(methylamino)pyridin-2-yl]ethanol (228 mg, 1.5 mmol) (WO2002088118), ethyl 3-(1,3-benzodioxol-5-yl)-4-(5-hydroxy-1-methyl-1H-pyrazol-3-yl)butanoate (498 mg, 1.5 mmol) (Example 1, Step 6), and triphenylphosphine (433 mg, 1.65 mmol) in anhydrous THF under N₂ gas at 0° C. was added diisopropyl azodicarboxylate (325 μL, 1.65 mmol). The reaction mixture was stirred at room temperature overnight. The reaction mixture was concentrated under reduced pressure. The residual oil was purified by reversed phase HPLC using (H₂O/TFA)/CH₃CN as eluent (2.5 mL TFA in 4 L H₂O) to afford 320 mg of the title compound as yellow oil.

Step 2. Synthesis of 3-(1,3-benzodioxol-5-yl)-4-(1-methyl-5-{2-[6-(methylamino)pyridin-2-yl]ethoxy}-1H-pyrazol-3-yl)butanoic acid.

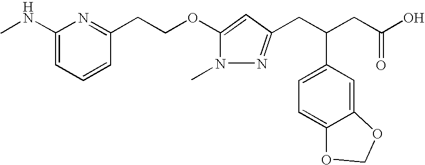

The product of step 1 (320 mg, 0.7 mmol) was dissolved in 3 ml methanol and 3 ml 1N sodium hydroxide solution. The reaction was stirred at room temperature overnight. the mixture was concentrated and acidified with 1 ml trifluoroacetic acid and purified by reversed phase HPLC using (H₂O/TFA)/CH₃CN as eluent (2.5 mL TFA in 4 L H₂O) to afford 117 mg of the title compound as yellow oil. FAB-MS:(MH⁺)=439. ¹H NMR (500 MHz, CD₃OD) δ 2.51-2.66 (m, 2H), 2.79 (m, 2H), 3.05 (s, 3H), 3.26 (t, 2H), 3.32 (m, 1H), 3.48 (s, 3H), 4.38 (t, 2H), 5.52 (s, 1H), 5.87 (s, 2H), 6.65-6.73 (m, 3H), 6.79 (d, 1H), 6.92 (d, 1H), 7.85 (t, 1H). Anal Calcd. for C₂₃H₂₆N₄O₅ plus 3 CF₃COOH and 1H₂O: C, 43.62; H, 3.91; N, 7.02. Found: 43.44; H, 4.15; N, 7.01.

### Example 9

### 3-(6-methoxypyridin-3-yl)-4-{1-methyl-5-[2-(5,6,7,8-tetrahydro-1,8-naphthyridin-2-yl)ethoxy]-1H-pyrazol-3-yl}butanoic acid

Step 1. Synthesis of 7-(2-bromoethyl)-1,2,3,4-tetrahydro-1,8-naphthyridine.

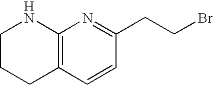

To a solution of (2-(5,6,7,8-tetrahydro-1,8-naphthyridin-2-yl)ethanol) (Example 1, Step 4) (1 g, 5.62 mmol) in benzene (20 mL) at room temperature under argon was added thionyl bromide (0.65 mL, 8.42 mmol) and the reaction mixture was stirred at 75° C. overnight. After cooling to room temperature the solvent was removed in vacuo. The dark oil was purified by chromatography on silica gel (eluent: 40:60 CH₂Cl₂/ethyl acetate) to yield the title compound.

Step 2. Synthesis of ethyl 3-(6-methoxypyridin-3-yl)-4-{1-methyl-5-[2-(5,6,7,8-tetrahydro-1,8-naphthyridin-2-yl)ethoxy]-1H-pyrazol-3-yl}butanoate.

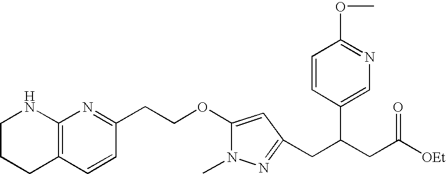

A mixture of the product of step 1 (265 mg, 1.1 mmol), DMF (10 mL), ethyl 4-(5-hydroxy-1-methyl-1H-pyrazol-3-yl)-3-(6-methoxypyridin-3-yl)butanoate (319 mg, 1 mmol) (Example 7, step 6) and K₂CO₃ (152 mg, 1.1 mmol) was heated to 60° C. overnight. The mixture was diluted with water, extracted with ethyl acetate. The ethyl acetate layer was washed with water, brine and then dried with Na₂SO₄. The solvent was removed and the residue was purified by reversed phase HPLC using (H₂O/TFA)/CH₃CN as eluent (2.5 mL TFA in 4 L H₂O) to afford 100 mg of the title compound as yellow oil. ¹H NMR (400 MHz, CD₃OD)δ1.15 (t, 3H), 1.98 (m, 2H), 2.65-2.95 (m, 6H), 3.18 (t, 2H), 3.45-3.54 (m, 6H), 3.99 (s, 3H), 4.02 (m, 2H), 4.37 (t, 2H), 5.61 (s, 1H), 6.70 (d, 1H), 7.04 (d, 1H), 7.61 (d, 1H), 7.90 (dd, 1H), 8.01 (d, 1H).

Step 3. Synthesis of 3-(6-methoxypyridin-3-yl)-4-{1-methyl-5-[2-(5,6,7,8-tetrahydro-1,8-naphthyridin-2-yl)ethoxy]-1H-pyrazol-3-yl}butanoic acid.

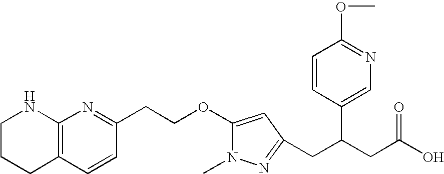

The product of step 2 (100 mg, 0.2 mmol) was dissolved in 3 ml methanol and 1.5 ml 1N sodium hydroxide solution. The reaction was stirred at room temperature overnight. The mixture was concentrated and acidified with 1 ml trifluoroacetic acid and purified by reversed phase HPLC using (H₂O/TFA)/CH₃CN as eluent (2.5 mL TFA in 4 L H₂O) to afford 58 mg of the title compound as yellow oil. FAB-MS:(MH⁺)=452. H1 NMR (500 MHz, CD₃OD) δ 1.94 (m, 2H), 2.60-2.90 (m, 4H), 3.15 (t, 2H), 3.43 (m, 4H), 3.50 (t, 2H), 3.94 (s, 3H), 4.30 (t, 2H), 5.51 (s, 1H), 6.68 (d, 1H), 6.92 (d, 1H), 7.60 (d, 1H), 7.81 (dd, 1H), 7.98 (d, 1H). Anal Calcd. for C₂₄H₂₉N₅O₄ plus 3.7 CF₃COOH and 1H₂O: C, 42.31; H, 3.92; N, 7.86. Found: 42.16; H, 4.26; N, 7.87.

### Example 10

### 3-(1,3-benzodioxol-5-yl)-4-{1-(2-hydroxyethyl)-5-[2-(5,6,7,8-tetrahydro-1,8-naphthyridin-2-yl)ethoxy]-1H-pyrazol-3-yl}butanoic acid

Step 1 Synthesis of diethyl 3-(1,3-benzodioxol-5-yl)-5-oxoheptanedioate.

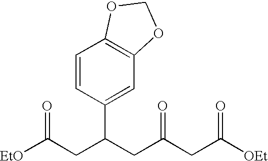

3-(1,3-benzodioxol-5-yl)-7-ethoxy-5,7-dioxoheptanoic acid (Example 1, Step 5) (4 g, 12.4 mmol) was dissolved in 4N HCl in ethanol and stirred at room temperature overnight. Concentrated under reduced pressure to afford 4.3 g of the title compound as a yellow oil.

Step 2. Synthesis of ethyl 3-(1,3-benzodioxol-5-yl)-4-[5-hydroxy-1-(2-hydroxyethyl)-1H-pyrazol-3-yl]butanoate.

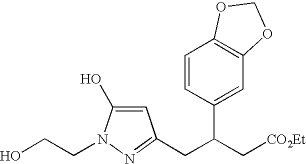

2-Hydroxyethylhydrazine (213 μL, 3.14 mmol) was added drop wise to a stirred solution of the product of step 1 (1 g, 2.85 mmol) in absolute ethanol (25 mL) at room temperature. The reaction mixture was stirred for 3 h. The solvent was removed under reduced pressure and the residue purified by flash column chromatography using 3% MeOH/CH₂Cl₂ as eluent to obtain a brown oil (750 mg). ¹H NMR (400 MHz, CDCl₃) δ1.20 (t, 3H), 2.55-2.83 (m, 4H), 3.37 (m, 1H), 3.75-3.84 (m, 4H), 4.05 (q, 2H), 5.95 (s, 2H), 6.63 (dd, 1H), 6.67 (d, 1H), 6.74 (d, 1H).

Step 3. Synthesis of ethyl 3-(1,3-benzodioxol-5-yl)-4-{1-(2-hydroxyethyl)-5-[2-(5,6,7,8-tetrahydro-1,8-naphthyridin-2-yl)ethoxy]-1H-pyrazol-3-yl}butanoate.

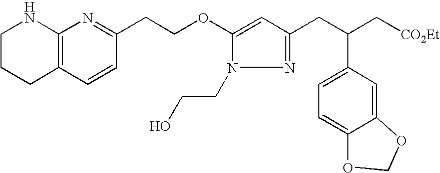

The title compound was prepared from the product of step 2 using the procedure described in Example 9, Step 2. H1 NMR (400 MHz, CD₃OD) δ1.13 (t, 3H), 1.96 (m, 2H), 2.56-2.95 (m, 6H), 3.18 (t, 2H), 3.37 (m, 1H), 3.52 (t, 2H), 3.71 (t, 2H), 4.01 (m, 4H), 4.44 (t, 2H), 5.73 (s, 1H), 5.88 (s, 2H), 6.65-6.76 (m, 4H), 7.60 (d, 1H).

Step 4. Synthesis of 3-(1,3-benzodioxol-5-yl)-4-{1-(2-hydroxyethyl)-5-[2-(5,6,7,8-tetrahydro-1,8-naphthyridin-2-yl)ethoxy]-1H-pyrazol-3-yl}butanoic acid.

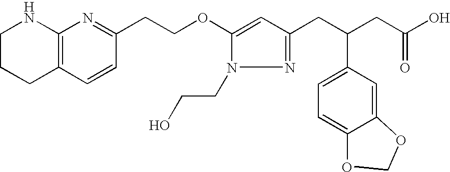

The product of step 3 (320 mg, 0.61 mmol) was dissolved in 3 ml methanol and 3 ml 1N sodium hydroxide solution. The reaction was stirred at room temperature overnight. The mixture was concentrated and acidified with 1 ml trifluoroacetic acid and purified by reversed phase HPLC using (H₂O/TFA)/CH₃CN as eluent (2.5 mL TFA in 4 L H₂O) to afford 180 mg of the title compound as yellow oil. FAB-MS:(MH⁺)=495. H1 NMR (500 MHz, CD₃OD) δ 1.95 (m, 2H), 2.50-2.68 (m, 2H), 2.74-2.85 (m, 4H), 3.14 (t, 2H), 3.35 (m, 1H), 3.50 (t, 2H), 3.70 (t, 2H), 3.92 (t, 2H), 4.34 (t, 2H), 5.50 (s, 1H), 5.87 (s, 2H), 6.65-6.74 (m, 4H), 7.60 (d, 1H). Anal Calcd. for C₂₆H₃₀N₄O₆ plus 2.4 CF₃COOH and 1H₂O: C, 47.05; H, 4.41; N, 7.13. Found: 47.04; H, 4.66; N, 6.97.

### Example 11

### (3S)-3-(1,3-benzodioxol-5-yl)-4-{1-(carboxymethyl)-5-[2-(5,6,7,8-tetrahydro-1,8-naphthyridin-2-yl)ethoxy]-1H-pyrazol-3-yl}butanoic acid

Step 1. Synthesis of ethyl (3S)-3-(1,3-benzodioxol-5-yl)-4-[1-(2-ethoxy-2-oxoethyl)-5-hydroxy-1H-pyrazol-3-yl]butanoate.

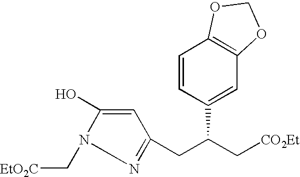

Ethyl hydrazinoacetate hydrochloric acid (969 mg, 6.27 mmol) was added to a stirred solution of diethyl (3S)-3-(1,3-benzodioxol-5-yl)-5-oxoheptanedioate (Example 5, Step 1) (2 g, 5.7 mmol) in absolute ethanol (25 mL) at room temperature. The reaction mixture was refluxed for 3 h. The reaction was cooled to room temperature and stirred overnight. The solvent was removed under reduced pressure and the residue purified by flash column chromatography using 5% MeOH/CH₂Cl₂ as eluent to obtain 410 mg of the title compound as a yellow oil. H1 NMR (400 MHz, CDCl₃) 11.18 (t, 3H), 1.26 (t, 3H), 2.55-2.68 (m, 3H), 2.79 (m, 1H), 3.35 (m, 1H), 4.05 (q, 2H), 4.20 (q, 2H), 4.38 (s, 2H), 5.30 (s, 1H), 5.95 (s, 2H), 6.63-6.75 (m, 3H).

Step 2. Synthesis of ethyl (3S)-3-(1,3-benzodioxol-5-yl)-4-{1-(2-ethoxy-2-oxoethyl)-5-[2-(5,6,7,8-tetrahydro-1,8-naphthyridin-2-yl)ethoxy]-1H-pyrazol-3-yl}butanoate.

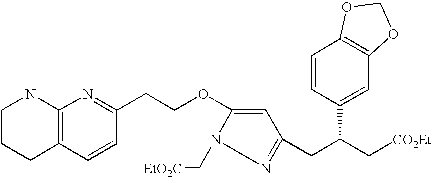

The title compound was prepared from the product of step 1 using the procedure described in Example 9, Step 2.

Step 3. Synthesis of (3S)-3-(1,3-benzodioxol-5-yl)-4-{1-(carboxymethyl)-5-[2-(5,6,7,8-tetrahydro-1,8-naphthyridin-2-yl)ethoxy]-1H-pyrazol-3-yl}butanoic acid.

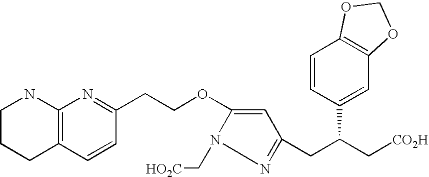

The product of step 2 (150 mg, 0.27 mmol) was dissolved in 3 ml methanol and 3 mL 1N sodium hydroxide solution. The reaction was stirred at room temperature overnight. The mixture was concentrated and acidified with 1 mL trifluoroacetic acid and purified by reversed phase HPLC using (H₂O/TFA)/CH₃CN as eluent (2.5 mL TFA in 4 L H₂O) to afford 82 mg of the title compound as white solid. FAB-MS:(MH⁺)=509. H1 NMR (500 MHz, CD₃OD) δ 1.94 (m, 2H), 2.47-2.66 (m, 2H), 2.70-2.84 (m, 4H), 3.12 (t, 2H), 3.30 (m, 1H), 3.50 (t, 2H), 4.34 (t, 2H), 4.57 (s, 2H), 5.45 (s, 1H), 5.87 (s, 2H), 6.65-6.74 (m, 4H), 7.58 (d, 1H). Anal Calcd. for C₂₆H₂₈N₄O₇ plus 2.1 CF₃COO: C, 48.49; H, 4.06; N, 7.49. Found: 48.26; H, 4.28; N, 7.74.

### Example 12

### 4-{1-methyl-5-[2-(5,6,7,8-tetrahydro-1,8-naphthyridin-2-yl)ethoxy]-1H-pyrazol-3-yl}-3-(2-phenyl-1,3-thiazol-5-yl)butanoic acid.

Step 1. Synthesis of 4-(2-phenyl-1,3-thiazol-5-yl)dihydro-2H-pyran-2,6(3H)-dione.

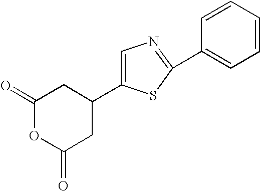

The anhydride was made according to the methods described for the preparation of 4-[2-(4-chlorophenyl)-1,3-thiazol-5-yl]dihydro-2H-pyran-2,6(3H)-dione (Example 2).

Step 2. Synthesis of 7-ethoxy-5,7-dioxo-3-(2-phenyl-1,3-thiazol-5-yl)heptanoic acid.

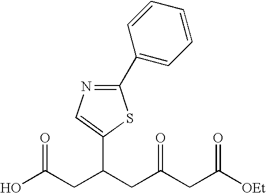

The title compound was prepared from the product of step 1 using the procedure described in Example 7, step 5.

Step 3. Synthesis of ethyl 4-(5-hydroxy-1-methyl-1H-pyrazol-3-yl)-3-(2-phenyl-1,3-thiazol-5-yl)butanoate.

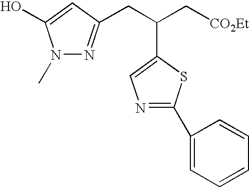

The title compound was prepared from the product of step 2 using the procedure described in Example 7, step 6. H1 NMR (400 MHz, CD₃OD) δ1.21 (t, 3H), 2.77-2.95 (m, 2H), 3.30-3.20 (m, 2H), 3.65 (s, 3H), 3.92 (m, 1H), 4.13 (q, 2H), 7.48 (m, 3H), 7.62 (s, 1H), 7.89 (m, 2H). Step 4. Synthesis of ethyl 4-{1-methyl-5-[2-(5,6,7,8-tetrahydro-1,8-naphthyridin-2-yl)ethoxy]-1H-pyrazol-3-yl}-3-(2-phenyl-1,3-thiazol-5-yl)butanoate.

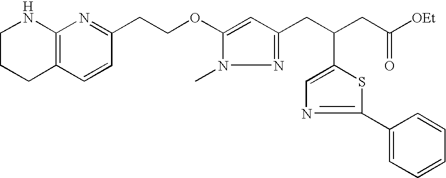

The title compound was prepared from the product of step 3 using the procedure described in Example 9, Step 2. H1 NMR (400 MHz, CD₃OD) δ1.20 (t, 3H), 1.85 (m, 2H), 2.68-3.01 (m, 6H), 3.15 (t, 2H), 3.50 (m, 5H), 3.86 (m, 1H), 4.09 (q, 2H), 4.36 (t, 2H), 5.63 (s, 1H), 6.65 (d, 1H), 7.45 (m, 3H), 7.54 (m, 2H), 7.85 (m, 2H).

Step 5. Synthesis of 4-{1-methyl-5-[2-(5,6,7,8-tetrahydro-1,8-naphthyridin-2-yl)ethoxy]-1H-pyrazol-3-yl}-3-(2-phenyl-1,3-thiazol-5-yl)butanoic acid.

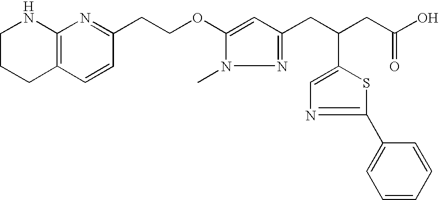

The product of step 4 (260 mg, 0.49 mmol) was dissolved in 3 ml methanol and 3 mL 1N sodium hydroxide solution. The reaction was stirred at room temperature overnight. The mixture was concentrated and acidified with 1 mL trifluoroacetic acid and purified by reversed phase HPLC using (H₂O/TFA)/CH₃CN as eluent (2.5 mL TFA in 4 L H₂O) to afford 130 mg of the title compound as yellow oil. FAB-MS:(MH⁺)=504. H1 NMR (500 MHz, CD₃OD) δ 1.94 (m, 2H), 2.66-3.00 (m, 6H), 3.13 (t, 2H), 3.48 (m, 5H), 3.82 (m, 1H), 4.34 (t, 2H), 5.56 (s, 3H), 4.30 (t, 2H), 6.64 (d, 1H), 7.44 (m, 3H), 7.55 (m, 2H), 7.84 (m, 2H). Anal Calcd. for C₂₇H₂₉N₅O₃S plus 2.7 CF₃COOH and 1H₂O: C, 46.91; H, 4.09; N, 8.44. Found: 46.91; H, 4.45; N, 8.53.

### Example 13

### 3-(6-methoxypyridin-3-yl)-4-{1-methyl-5-[2-(5,6,7,8-tetrahydro-1,8-naphthyridin-2-yl)ethoxy]-1H-pyrazol-3-yl}butanoic acid.

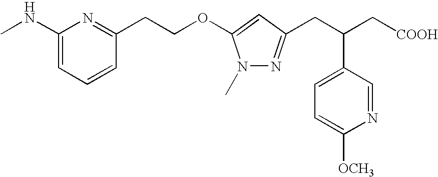

A mixture of triphinylphosphine, isopropyl diazodicarboxylate, and THF was stirred at 0° C. for 30 minutes. Ethyl 3-(6-methoxypyridin-3-yl)-4-(1-methyl-5-oxo-4,5-dihydro-1H-pyrazol-3-yl) butanoate (Example 7, Step 6) was added, following the addition of 2-[6-(methylamino)pyridin-2-yl]ethanol (WO2002088118). The resulting solution was warmed to room temperature and stirred overnight. The reaction mixture was concentrated in vacuo and purified via reverse phase HPLC using a gradient of 10-50% acetonitrile/ H₂O/0.05% TFA over 30 min to gain the acetate intermediate. This intermediate was stirred at in a mixture of 1N NaOH aqueous (15 mL) and ethanol (30 ml). The reaction was concentrated and purified via reverse phase HPLC, using the gradient of 5-50% acetonitrile/ H₂O/0.05% TFA over 30 min to yield 126 mg desired product. ¹H NMR (CD3CN) δ 8.00 (d 1H), 7.65-7.85 (m, 2H), 6.90 (d, 1H), 6.80 (d, 1H), 6.70 (d, 1H), 5.62 (s, 1H), 4.20 (t, 2H), 4.95 (s, 3H), 3.45 (s, 3H), 3.40 (m, 1H), 3.20 (t, 2H), 2.95 (s, 3H), 2.95 (m, 2H), 2.65 (m, 2H). Analysis Calculated for C₂₂H₂₇F₂N₅O₄4.5 TFA. Expected: C, 38.29; H, 3.53; N, 7.32. Found: C, 38.82; H, 3.81; N, 7.59. Calculated Mass: 425 Found Mass: 426 (for MH+).

### Example 14

### 4-{1-(4-Cyanophenyl)-5-[2-(5,6,7,8-tetrahydro-1,8-naphthyridin-2-yl)ethoxy]-1H-pyrazol-3-yl}-3-(6-methoxypyridin-3-yl)butanoic acid.

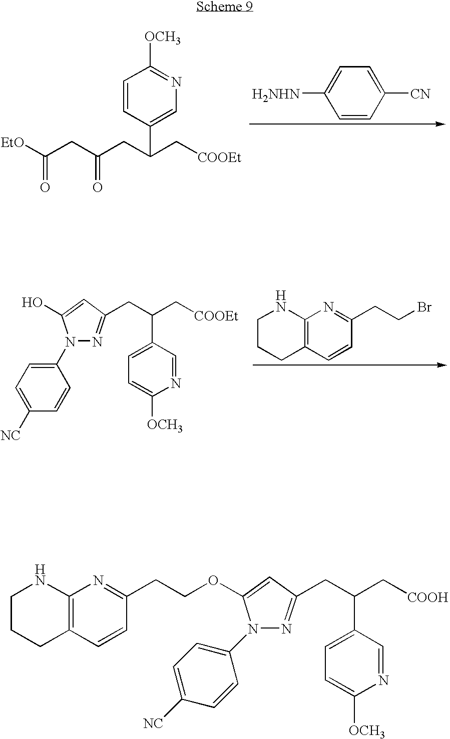

Step 1. Ethyl 4-[1-(4-cyanophenyl)-5-oxo-4,5-dihydro-1H-pyrazol-3-yl]-3-(6-methoxypyridin-3-yl)butanoate.

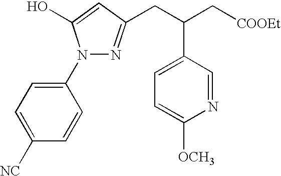

The title compound was prepared from diethyl 3-(6-methoxypyridin-3-yl)-5-oxoheptanedioate (600 mg, 1.71 mmol) (Example 7, Step 5) and 4-cyanophenyl hydrazine using the procedure described in Example 7, Step 6. The crude product was purified by flash chromatography (30% EA/Hex) to give 0.429 g desired product. ¹H NMR (CDCl₃) δ 8.00 (d, 2H), 7.68 (t, 2H), 6.75 (m, 3H), 6.95 (s, 2H), 6.92 (s, 1H), 4.03 (m, 2H), 3.45 (m, 1H), 2.85 (m, 2H), 2.68 (m, 2H), 1.20 (t, 3H).

Step 2. 4-{1-(4-Cyanophenyl)-5-[2-(5,6,7,8-tetrahydro-1,8-naphthyridin-2-yl)ethoxy]-1H-pyrazol-3-yl}-3-(6-methoxypyridin-3-yl)butanoic acid

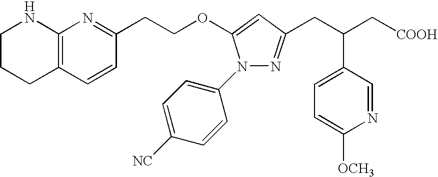

A mixture of the product of step 1,7-(2-bromoethyl)-1,2,3,4-tetrahydro-1,8-naphthyridine, K2CO3, and DMF was heated to 60° C. overnight. The reaction was concentrated and purified via reverse phase HPLC, using the gradient of 10-50% acetonitrile/ H₂O/0.05% TFA over 30 min. This material was stirred in a mixture of 1N NaOH aqueous (20 ml) and ethanol (30 ml) overnight. The reaction was concentrated and purified via reverse phase HPLC, using the gradient of 10-50% acetonitrile/ H₂O/0.05% TFA over 30 min to give 180 mg yellow solid product. ¹H NMR (CD₃OD) δ 7.67 (m, 4H), 7.60 (d, 1H), 6.80 (s, 1H), 6.77 (s, 1H), 6.67 (d, 1H), 5.95 (s, 2H), 5.67 (s, 1H), 4.40 (m, 2H), 3.50(m, 2H), 3.30 (s, 3H), 3.20 (m, 2H), 2.95 (m, 2H), 2.80 (m, 2H), 2.62 (m, 2H), 1.95 (m, 2H). Analysis Calculated for C₃₁H₂₉N₅O₅ 2.2 TFA 0.7H₂O. Expected: C, 52.17; H, 4.03; N, 8.59. Found: C, 52.05; H, 4.15; N, 8.61. Calculated Mass: 551. Found Mass: 552 (for MH+).

### Example 15

### 4-{1-[4-(Aminosulfonyl)phenyl]-5-[2-(5,6,7,8-tetrahydro-1,8-naphthyridin-2-yl)ethoxy]-1H-pyrazol-3-yl}-3-(6-methoxypyridin-3-yl)butanoic acid

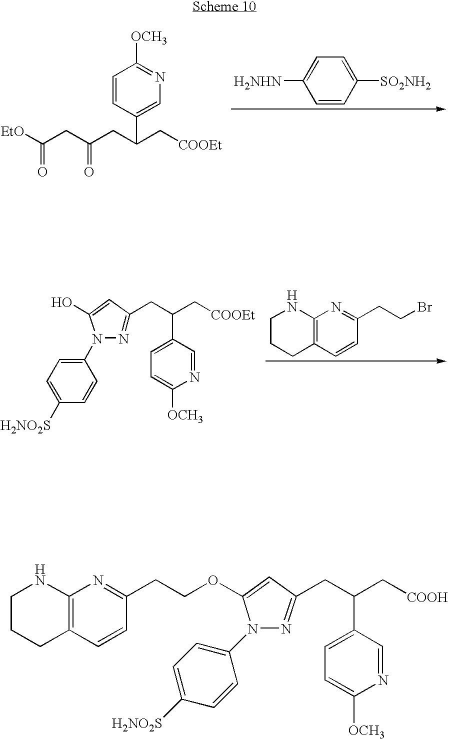

The title compound was prepared as described in Example 14, using 4-aminosulfonylphenyl hydrazine in place of 4-cyanophenyl hydrazine. ¹H NMR (DMSO) δ 8.30 (s, 1H), 7.80 (d, 2H), 7.67 (d, 2H), 7.60 (d, 1H), 7.40 (s, 2H), 6.80 (s, 1H), 6.77 (d, 1H), 6.87 (m, 2H), 5.95 (s, 2H), 5.80 (s, 1H), 4.40 (m, 2H), 3.43(m, 2H), 3.36 (m, 1H), 3.20 (m, 2H), 2.80 (m, 2H), 2.65 (m, 2H), 2.62 (m, 2H), 1.90 (m, 2H). Analysis Calculated for C₃₀H₃₁N₅O₇S 1.5 TFA 1.0H₂O. Expected: C, 49.87; H, 4.38; N, 8.81. Found: C, 49.49; H, 4.58; N, 9.09. Calculated Mass: 605. Found Mass: 606 (for MH+).

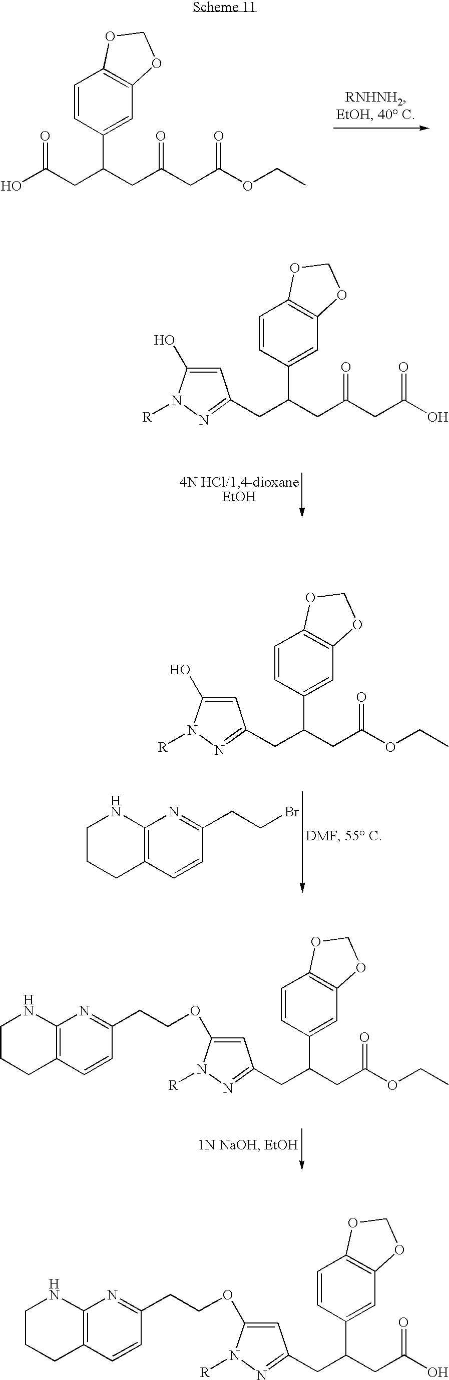

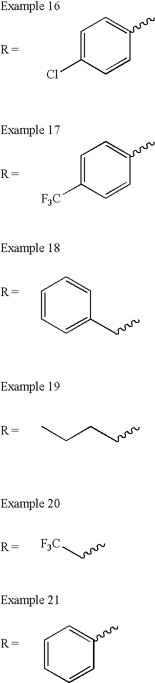

### Example 16

### 3-(1,3-benzodioxol-5-yl)-4-{1-(4-chlorophenyl)-5-[2-(5,6,7,8-tetrahydro-1,8-naphthyridin-2-yl)ethoxy]-1H-pyrazol-3-yl}butanoic acid.

Step 1. Synthesis of 2 (3-(1,3-benzodioxol-5-yl)-4-[1-(4-chlorophenyl)-5-hydroxy-1H-pyrazol-3-yl]butanoic acid.

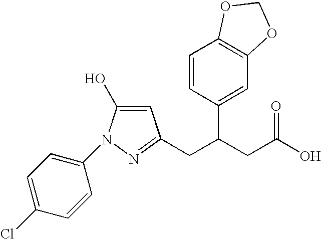

To a solution of 3-(1,3-benzodioxol-5-yl)-7-ethoxy-5,7-dioxoheptanoic acid (1.5 g, 4.65 mmol) in ethanol (40 mL) at 40° C. under argon was added 4-chlorophenyl hydrazine hydrochloride (0.917 g, 5.11 mmol). The reaction was refluxed for 3.5 hours. The reaction mixture was concentrated in vacuo to reduce the volume to 20 mL.

Step 2. Synthesis of ethyl 3-(1,3-benzodioxol-5-yl)-4-[1-(4-chlorophenyl)-5-hydroxy-1H-pyrazol-3-yl]butanoate).

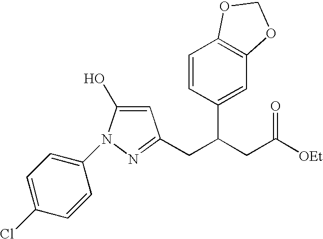

To a solution of the product from Step 1 in ethanol (20 mL) at room temperature under argon was added 4N HCl/ dioxane solution (15 mL) and the mixture stirred for 6.5 hours. The volatiles were removed in vacuo. The mixture was extracted three times with ethyl acetate and all organic extracts were combined, washed with brine, dried over MgSO₄, and concentrated under reduced pressure to get the crude product, which was purified by chromatography on silica gel (eluent: 25% ethyl acetate/hexane). The desired product is a yellow semi-solid (0.636 g, 35%).

Step 3. Synthesis of ethyl 3-(1,3-benzodioxol-5-yl)-4-{1-(4-chlorophenyl)-5-[2-(5,6,7,8-tetrahydro-1,8-naphthyridin-2-yl)ethoxy]-1H-pyrazol-3-yl}butanoate.

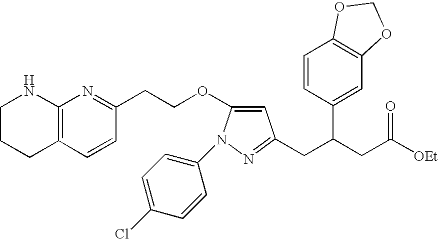

To a solution of the product from Step 2 (0.636 g, 1.483 mmol) and K₂CO₃ (0.245 g, 1.78 mmol) in DMF (20 mL) at 55° C. under argon was added 7-(2-bromoethyl)-1,2,3,4-tetrahydro-1,8-naphthyridine (0.358 g, 1.483 mmol) and the mixture stirred at 55° C. for 4 hours. The volatiles were removed in vacuo. The mixture was extracted three times with ethyl acetate and all organic extracts were combined, dried over MgSO₄, and concentrated under reduced pressure to give the crude product, which was purified by residue was purified using reverse phase HPLC with acetonitrile gradient 5-50% in 30 min to yield 0.2 g product as a yellow oil.

Step 4. Synthesis of 3-(1,3-benzodioxol-5-yl)-4-{1-(4-chlorophenyl)-5-[2-(5,6,7,8-tetrahydro-1,8-naphthyridin-2-yl)ethoxy]-1H-pyrazol-3-yl}butanoic acid.

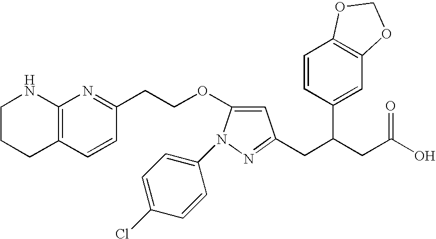

The product of Step 3 (0.2 g, 0.34 mmol) was dissolved in EtOH (2 mL) and 1N NaOH solution (1.1 mL) and stirred overnight under argon at room temperature. The reaction was concentrated and crude product was purified using reverse phase HPLC with acetonitrile gradient 5-50% in 30 min to yield the product (0.100 g) as a foamy yellow solid. FAB-MS:(MH⁺)=561.19. ¹H NMR (500 MHz, CD₃OD) δ1.93 (m, 2H), 2.58 (m, 1H), 2.70 (m, 1H), 2.83 (m, 6H), 3.14 (t, 2H), 3.42 (m, 1H), 3.48 (t, 2H), 4.42 (t, 2H), 5.67 (s, 1H), 5.89 (s, 2H), 6.58 (d, 1H), 6.72 (s, 2H), 6.78 (s, 1H), 7.4 (s, 4H), 7.53 (d, 1H). Anal Calcd. for C₃₀H₂₉ClN₄O₅ plus 1.4 CF₃COOH, 2.6H₂O: C, 51.33; H, 4.68; N, 7.30. Found: 51.19; H, 5.07; N, 7.04.

### Example 17

### 3-(1,3-benzodioxol-5-yl)-4-{5-[2-(1,2,3,4-tetrahydropyrido[2,3-b]pyrazin-6-yl)ethoxy]-1-[4-(trifluoromethyl)phenyl]-1H-pyrazol-3-yl}butanoic acid.

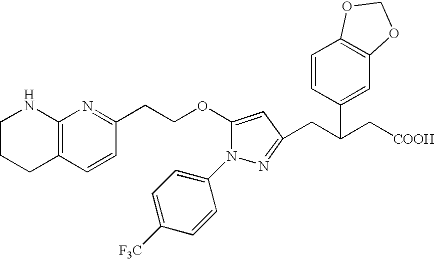

The title compound was prepared as described in Example 16 using 4-(trifluoromethyl)phenyl hydrazine in place of 4-chlorophenyl hydrazine hydrochloride. FAB-MS:(MH⁺)=595.21. ¹H NMR (500 MHz, CD₃OD) δ 1.91 (m, 2H), 2.59 (m, 1H), 2.72 (m, 1H), 2.77 (t, 2H), 2.84 (m, 1H), 2.63 (m, 1H), 2.90 (m, 1H), 3.18 (t, 2H), 3.42 (m, 1H), 3.47 (t, 2H), 4.47 (t, 2H), 5.68 (s, 1H), 5.89 (s, 2H), 6.6 (d, 1H), 6.74 (s, 2H), 6.78 (s, 1H), 7.50 (d, 1H), 7.7 (s, 4H). Anal Calcd. for C₃₁H₂₉F₃N ₄O₅ plus 1.4 CF₃COOH, 1.0H₂O: C, 52.57; H, 4.23; N, 7.26. Found: 52.35; H, 4.44; N, 7.01.

### Example 18

### 3-(1,3-benzodioxol-5-yl)-4-{1-benzyl-5-[2-(5,6,7,8-tetrahydro-1,8-naphthyridin-2-yl)ethoxy]-1H-pyrazol-3-yl}butanoic acid.

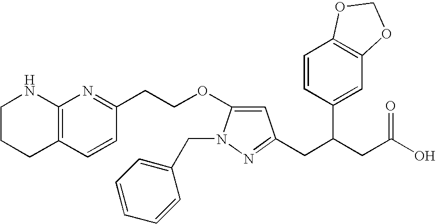

The title compound was prepared as described in Example 16 using benzyl hydrazine oxalate in place of 4-chlorophenyl hydrazine hydrochloride to obtain a foamy yellow solid. FAB-MS:(MH⁺)=541.25. ¹H NMR (400 MHz, CD₃OD) δ 1.88 (m, 2H), 2.55 (m, 1H), 2.65 (m, 1H), 2.75 (m, 1H), 2.82 (m, 3H), 3.2 (t, 2H), 3.35 (m, 1H), 3.44 (t, 2H), 4.32 (t, 2H), 4.98 (s, 2H), 5.54 (s, 1H), 5.83 (m, 2H), 6.48 (d, 1H), 6.67 (m, 6H), 7.2 (m, 2H), 7.42 (d, 1H). Anal Calcd. for C₃₁H₃₂N₄O₅ plus 2.2 CF₃COOH, 1.0H₂O: C,52.53; H, 4.51; N, 6.92. Found: 52.42; H, 4.92; N, 6.74.

### Example 19

### 3-(1,3-benzodioxol-5-yl)-4-{1-butyl-5-[2-(5,6,7,8-tetrahydro-1,8-naphthyridin-2-yl)ethoxy]-1H-pyrazol-3-yl}butanoic acid.

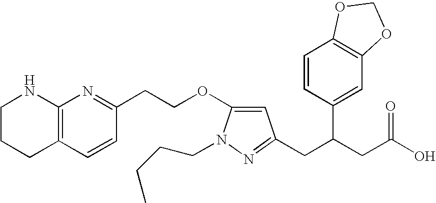

The title compound was prepared as described in Example 16 using butyl hydrazine oxalate in place of 4-chlorophenyl hydrazine hydrochloride. FAB-MS:(MH⁺)=507.26. ¹H NMR (400 MHz, CD₃OD) δ 0.81 (t, 3H), 1.70 (m, 2H), 1.24 (t, 1H), 1.53 (m, 2H), 1.95 (m, 2H), 2.54 (m, 1H), 2.63 (m, 1H), 2.74 (m, 1H), 2.82 (m, 3H), 3.16 (t, 2H), 3.30 (m, 1H), 3.50 (t, 2H), 3.78 (t, 2H), 4.32 (t, 2H), 5.47 (s, 1H), 5.86 (s, 2H), 6.65 (m, 4H), 7.6 (d, 1H). Anal Calcd. for C₂₈H₃₆N₄O₅ plus 1.5 CF₃COOH, 0.5H₂O: C, 54.23; H, 5.36; N, 7.63. Found: 54.40; H, 5.82; N, 7.63.

### Example 20

### 3-(1,3-benzodioxol-5-yl)-4-[5-[2-(5,6,7,8-tetrahydro-1,8-naphthyridin-2-yl)ethoxy]-1-(2,2,2-trifluoroethyl)-1H-pyrazol-3-yl]butanoic acid.

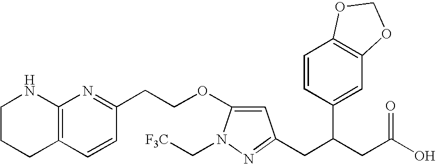

The title compound was prepared as described in Example 16 using a 70% solution of 2,2,2-trifluoroethyl hydrazine in water in place of 4-chlorophenyl hydrazine hydrochloride. FAB-MS:(MH⁺)=533.20. ¹H NMR (400 MHz, CD₃OD) δ1.95 (m, 2H), 2.54 (m, 1H), 2.65 (m, 1H), 2.75 (m, 1H), 2.82 (m, 3H), 3.15 (t, 2H), 3.33 (m, 1H), 3.5 (t, 2H), 4.35 (t, 2H), 4.5 (m, 2H), 5.5 (s, 1H), 5.86 (s, 2H), 6.66 (m, 4H), 7.6 (d, 1H). Anal Calcd. for C₂₆H₂₇F₃N₄O₅ plus 1.0 CF₃COOH, 1.2H₂O: C, 49.84; H, 4.52; N, 8.24. Found: 50.24; H, 4.77; N, 7.64.

### Example 21

### 3-Benzo[1,3]dioxol-5-yl-4-{1-benzyl-5-[2-(5,6,7,8-tetrahydro-[1,8] naphthyridin-2-yl)-ethoxy]-1H-pyrazol-3-yl}-butyric acid.

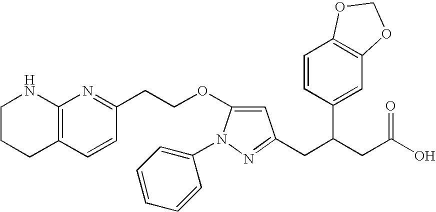

The title compound was prepared as described in Example 16 using phenyl hydrazine in place of 4-chlorophenyl hydrazine hydrochloride to obtain a brown color oil. Anal. MS (APCI): m/z=527 (MH⁺), ¹H-NMR (CD₃CN): δ 1.85 (2H, m, CH₂), δ 2.54 and δ 2.24 (2H, qq, CH₂), δ 2.71 (2H, t, CH₂), δ 2.82 (2H, m, CH₂)δ 3.09 (2H, t, CH₂), δ 3.38 (1H, m, CH), δ 3.42 (2H, t, CH₂)δ 4.36 (2H, t, CH₂)δ 5.59 (1H, s, CH), δ 6.46 (1H, d, CH), δ 6.72 (2H, t, 2XCH), δ 6.79 (1H, s, CH), δ 7.25 (1H, t, CH), δ 7.38 (3H, m, 3XCH), δ 7.47 (2H, d, 2XCH). Calcd. for C₃₀H₃₀N₄O₅2.0 TFA, 0.5H₂O: C, 53.48; H, 4.36; N, 7.34, Found: C, 53.37; H, 4.57; N, 7.21.

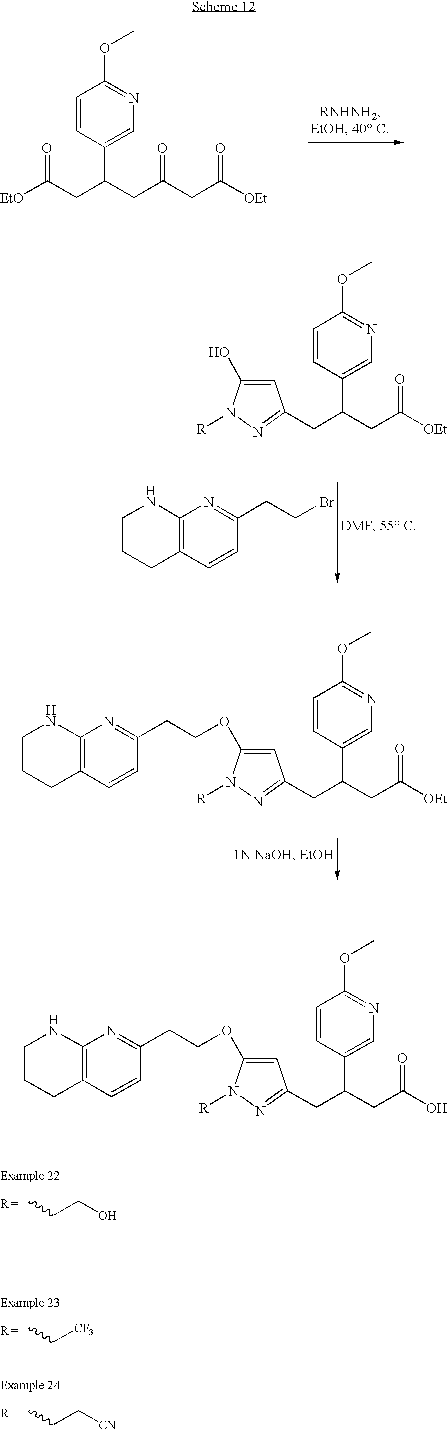

### Example 22

### 4-{1-(2-hydroxyethyl)-5-[2-(5,6,7,8-tetrahydro-1,8-naphthyridin-2-yl)ethoxy]-1H-pyrazol-3-yl}-3-(6-methoxypyridin-3-yl)butanoic acid.

Step 1. Synthesis of ethyl 4-[5-hydroxy-1-(2-hydroxyethyl)-1H-pyrazol-3-yl]-3-(6-methoxypyridin-3-yl)butanoate.

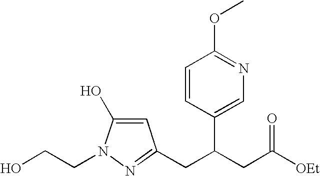

To a solution of diethyl 3-(6-methoxypyridin-3-yl)-5-oxoheptanedioate (2.0 g, 5.93 mmol) (prepared from 7-ethoxy-3-(6-methoxypyridin-3-yl)-5,7-dioxoheptanoic acid using the procedure described in Example 10, Step 1) in ethanol (5 mL) at room temperature under argon was added 2-hydroxyethyl hydrazine (0.44 mL, 6.52 mmol) and the reaction was stirred at room temperature for 4 hours. The reaction mixture was concentrated in vacuo to afford the product as a yellow solid.

Step 2. Synthesis of ethyl 4-{1-(2-hydroxyethyl)-5-[2-(5,6,7,8-tetrahydro-1,8-naphthyridin-2-yl)ethoxy]-1H-pyrazol-3-yl}-3-(6-methoxypyridin-3-yl)butanoate.

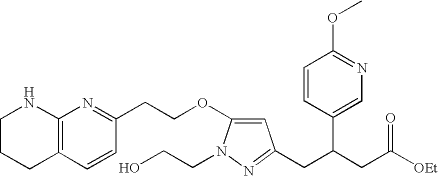

To a solution of the product of Step 1 (1.0 g, 2.86 mmol) and K₂CO₃ (0.47 g, 3.53 mmol) in DMF (5 mL) at 55° C. under argon was added (7-(2-bromoethyl)-1,2,3,4-tetrahydro-1,8-naphthyridine) (0.69 g, 3.53 mmol) and the mixture stirred at 55° C. for 8 hours. The volatiles were removed in vacuo. The mixture was extracted three times with ethyl acetate and all organic extracts were combined, dried over MgSO₄, and concentrated under reduced pressure to give the crude product, which was purified by residue was purified using reverse phase HPLC with acetonitrile gradient 5-50% in 30 min to yield 0.3 g product as a yellow solid.

Step 3. Synthesis of 3-(1,3-benzodioxol-5-yl)-4-{1-benzyl-5-[2-(5,6,7,8-tetrahydro-1,8-naphthyridin-2-yl)ethoxy]-1H-pyrazol-3-yl}butanoic acid.

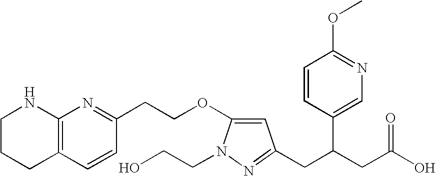

The product of step 2 (3 g, 0.59 mmol) was dissolved in EtOH (4.5 mL) and 1N NaOH solution (2.5 mL) and stirred overnight under argon at room temperature. The reaction was concentrated and crude product was purified using reverse phase HPLC with acetonitrile gradient 1-50% in 30 min to yield 0.11 g of product as a yellow solid. FAB-MS:(MH⁺)=482.24. ¹H NMR (500 MHz, CD₃OD) δ1.95 (m, 2H), 2.60 (m, 1H), 2.80 (m, 6H), 3.15 (t, 2H), 3.41 (m, 1H), 3.51 (t, 2H), 3.69 (t, 2H), 3.90 (s, 3H), 4.34 (t, 2H), 5.50 (s, 1H), 6.70 (d, 1H), 6.83 (d, 1H), 7.61 (d, 1H), 7.70 (dd, 1H), 7.93 (d, 1H), 8.11 (s, 1H). Anal Calcd. for C₂₅H₃₁N 505 plus 1.7 CF₃COOH, 1.0H₂O, 0.2 CH₃CN: C, 49.30; H, 5.09; N, 10.38. Found: 49.50; H, 5.09; N, 9.94.

### Example 23

### 3-(6-methoxypyridin-3-yl)-4-[5-[2-(5,6,7,8-tetrahydro-1,8-naphthyridin-2-yl)ethoxy]-1-(2,2,2-trifluoroethyl)-1H-pyrazol-3-yl]butanoic acid.

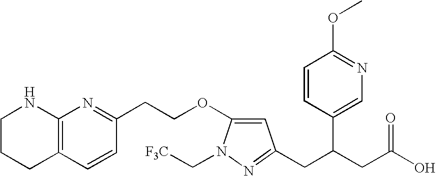

The title compound was prepared as described in Example 22 using a 70% solution of 2,2,2-trifluoroethyl hydrazine in water in place of the 2-hydroxyethyl hydrazine. FAB-MS:(MH⁺)=520.23. ¹H NMR (500 MHz, CD₃OD) δ1.94 (m, 2H), 2.65 (m, 1H), 2.75 (m, 1H), 2.88 (t, 2H), 2.90 (m, 1H), 3.15 (t, 2H), 3.48 (m, 1H), 3.5 (t, 2H), 3.9 (s, 3H), 4.35 (t, 2H), 4.48 (q, 2H), 5.56 (s, 1H), 6.68 (d, 1H), 6.86 (d, 1H), 7.73 (dd, 1H), 7.93 (d, 1H). Anal Calcd. for C₂₆H₂₇F₃N₄O₅ plus 1.0 CF₃COOH, 1.2H₂O: C,49.84; H, 4.52; N, 8.24. Found: 50.24; H, 4.77; N, 7.64. Anal Calcd. for C₂₅H₂₈F₃N 504 plus 2.4 CF₃COOH, 1.0H₂O: C, 44.12; H, 4.03; N, 8.63. Found: 44.48; H, 4.24; N, 8.76.

### Example 24

### 4-{1-(2-cyanoethyl)-5-[2-(5,6,7,8-tetrahydro-1,8-naphthyridin-2-yl)ethoxy]-1H-pyrazol-3-yl}-3-(6-methoxypyridin-3-yl)butanoic acid.

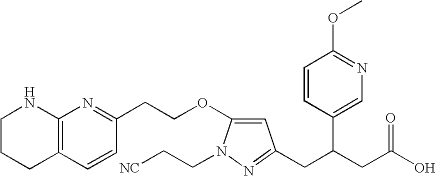

The title compound was prepared as described in Example 22 using 4-cyanoethyl hydrazine hydrochloride in place of the 2-hydroxyethyl hydrazine. FAB-MS:(MH⁺)=491.13. ¹H NMR (500 MHz, CD₃OD) δ1.94 (m, 2H), 2.65 (m, 1H), 2.75 (m, 6H), 2.92 (m, 1H), 3.17 (t, 2H), 3.45 (m, 1H), 3.49 (t, 2H), 3.92 (s, 3H), 4.04 (t, 2H), 4.35 (t, 2H), 5.51 (s, 1H), 6.70 (d, 1H), 6.90 (d, 1H), 7.58 (d, 1H), 7.75 (dd, 1H), 7.95 (d, 1H). Anal Calcd. for C₂₆H₃₀N 604 plus 1.5 CF₃COOH, 2.0H₂O: C, 48.33; H, 4.73; N, 11.27. Found: 48.10; H, 4.35; N, 11.48.

### Example 25

### 3-(1,3-benzodioxol-5-yl)-4-{1-methyl-5-[2-(1,2,3,4-tetrahydropyrido[2,3-b]pyrazin-6-yl)ethoxy]-1H-pyrazol-3-yl}butanoic acid.

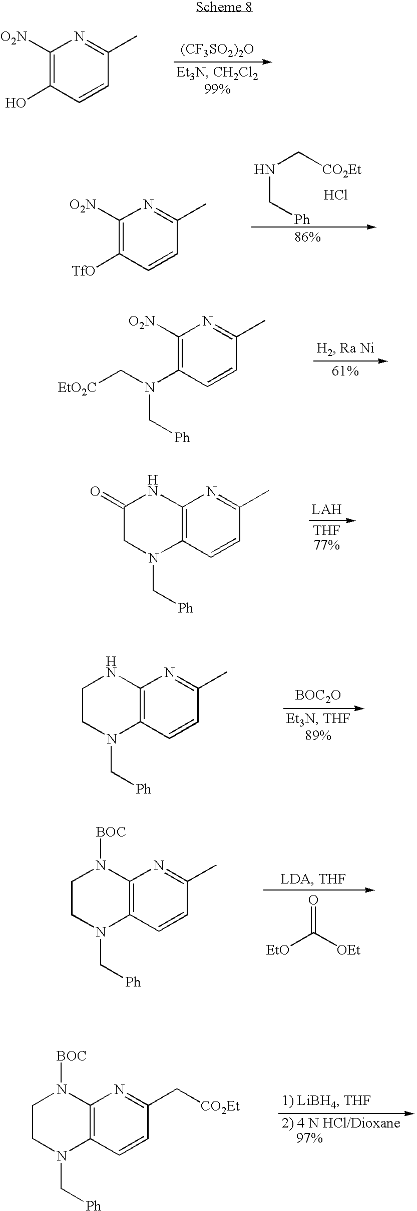

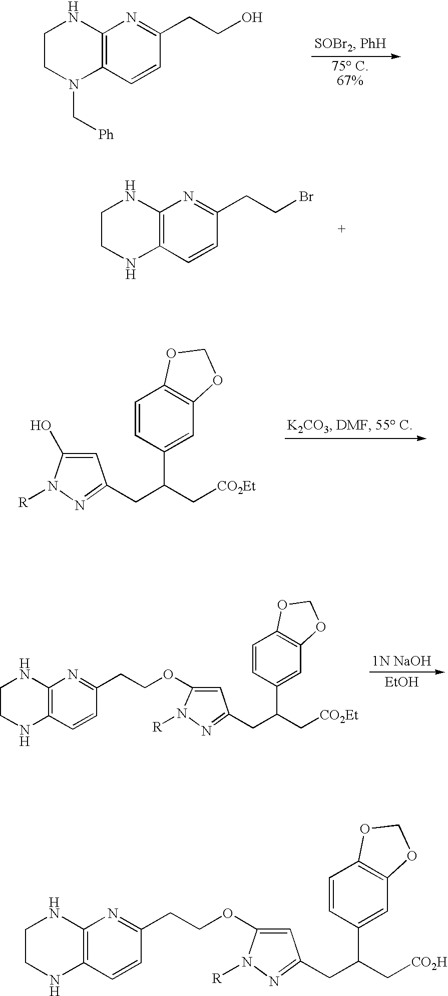

Step 1. Synthesis of 6-methyl-2-nitropyridin-3-yl trifluoromethanesulfonate.

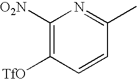

To a solution of 3-hydroxy-6-methyl-2-nitropyridine (10 g, 64.88 mmol) in CH₂Cl₂ (300 mL) at 0° C. under argon was added triethylamine (13.44 mL, 96.41 mmol) and followed by trifluoromethanesulfonic anhydride (13.1 mL, 77.86 mmol). The mixture was stirred for 2 hours at 0° C. and then quenched with water. The organic layer was separated, washed with water and dried over MgSO₄. After filtration and concentration at reduced pressure, the crude mixture was purified by flash chromatography on silica gel (15% EA/Hex) to afford the desired product (18.4 g, 99%) as a yellow oil. H1 NMR (400 MHz, CDCl₃) δ 2.70 (s, 3H), 7.59 (d, 1H), 7.81(d, 2H).

Step 2. Synthesis of ethyl N-benzyl-N-(6-methyl-2-nitropyridin-3-yl)glycinate.

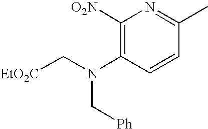

To the product of step 1 (14 g, 48.92 mmol) at room temperature under argon was added N-benzylglycine ethyl ester (18.91 g, 97.84 mmol). The mixture was stirred at 95° C. under argon for 16 hours and additional N-benzylglycine ethyl ester (3.0 g, 15.52 mmol) was added to the reaction mixture and stirred for 4 hours. The reaction was cooled to room temperature and the crude mixture was purified by flash chromatography on silica gel (10-15% EA/Hex) to afford the desired product (13.8 g, 86%) as yellow oil. H1 NMR (400 MHz, CDCl₃) δ 1.12 (t, 3H), 2.38 (s, 3H), 3.62 (s, 2H), 4.20 (q, 2H), 4.38 (s, 2H), 7.3(m, 5H), 7.5 (s, 1H), 7.72 (s, 1H).

Step 3. Synthesis of 1-benzyl-6-methyl-1,4-dihydropyrido[2,3-b]pyrazin-3(2H)-one.

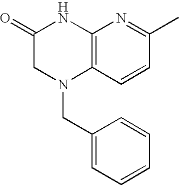

The product of step 2 (13.0 g, 39.47 mmol) was hydrogenated in ethanol solution at room temperature using H₂ at 5 psi and Raney Nickel catalyst for 72 hours. Upon completion of the reaction, the catalyst was filtered off and the filtrate was concentrated under reduced pressure. The product was crystallized from ethyl acetate as a yellow crystalline solid. The mother liquor was concentrated and purified by flash chromatography on silica gel (25% EA/Hex) to yield the desired product (6.1 g, 61%). H1 NMR (400 MHz, CDCl₃) δ 2.37 (s, 3H), 2.78 (s, 2H), 4.32 (s, 2H), 6.63 (d, 1H), 6.80 (d, 1H), 7.25 (m, 5H).

Step 4. Synthesis of 1-benzyl-6-methyl-1,2,3,4-tetrahydropyrido[2,3-b]pyrazine.

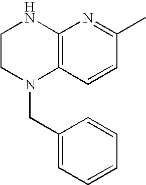

To a solution of the product of step 3 (2.5 g, 9.88 mmol) in anhydrous THF (40 mL) in a round-bottom flask fitted with a stir bar and a condenser was slowly added a 1M solution of LiAlH₄ (19.76 mL, 19.76 mmol) in THF. Upon completion of the addition, the reaction mixture was refluxed for 16 hours. The reaction was cooled to room temperature and quenched with 1M NaOH solution until the mixture had become a milky yellow color. After stirring for 5 minutes, the precipitate was filtered off and washed 3 times with CH₂Cl₂. The filtrate and washings were combined, washed with brine, dried over MgSO₄, filtered and concentrated in vacuo to yield the desired product (1.8 g, 77%). H1 NMR (400 MHz, CDCl₃) δ2.32 (s, 3H), 3.34 (t, 2H), 3.60 (t, 2H), 4.38 (s, 2H), 6.33 (d, 1H), 6.58 (d, 2H), 7.3 (m, 5H).

Step 5. Synthesis tert-butyl 1-benzyl-6-methyl-2,3-dihydropyrido[2,3-b]pyrazine-4(1H)-carboxylate.

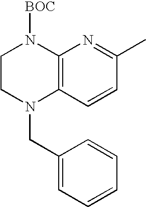

A solution of product of step 4 (1.8 g, 7.53 mmol), di-tert-butyl dicarbonate (2.46 g, 11.29 mmol), DMAP (0.09 g, 0.75 mmol) and triethylamine (1.14 mL, 11.29 mmol) in THF (30 mL) was refluxed under argon for 72 hours. The reaction mixture was cooled to room temperature, diluted with ethyl acetate and was washed with brine, dried over Na₂SO₄. After filtration and concentration under reduced pressure, the crude mixture was purified by flash chromatography on silica gel (20% EA/Hex) to afford the desired product (2.28 g, 89%). H1 NMR (400 MHz, CDCl₃) δ 1.34 (s, 9H), 2.40 (s, 3H), 3.2 (t, 2H), 3.65 (t, 2H), 4.28 (s, 2H), 6.51 (d, 1H), 6.58 (d, 2H), 7.18 (m, 5H).

Step 6. Synthesis of tert-butyl 1-benzyl-6-(2-ethoxy-2-oxoethyl)-2,3,4a,8a-tetrahydropyrido[2,3-b]pyrazine-4(1H)-carboxylate.

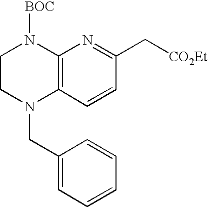

A2.0 M solution of Lithium diisopropylamide (10.1 mL, 20.15 mmol) in THF/ethylbenzene/heptane was added drop wise to a chilled (−78° C.), stirred solution of the product of step 5 (2.28, 6.72 mmol) and diethyl carbonate (3.0 mL, 25.0 mmol) in anhydrous THF (35 mL) under argon atmosphere. After 1 hour the reaction was quenched with saturated NH₄Cl solution and warmed to room temperature. The mixture was extracted three times with ethyl acetate and all organic extracts were combined, dried over Na₂SO₄, and concentrated under reduced pressure to yield the desired product (3.0 g) as a yellow solid. H1 NMR (400 MHz, CDCl₃) δ 1.25 (t, 3H), 1.42 (s, 9H), 3.32 (t, 2H), 3.78 (t, 2H), 4.05 (q, 2H), 4.4 (s, 2H), 6.75 (m, 2H), 7.18 (m, 5H).

Step 7. Synthesis of 2-(1-benzyl-1,2,3,4-tetrahydropyrido[2,3-b]pyrazin-6-yl)ethanol.

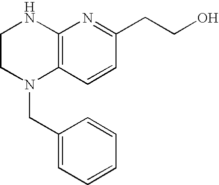

To a solution of the product of step 6 (3.0 g, 7.29 mmol)) in dry THF (30 mL) at room temperature was added a solution of LiBH₄ (2.0 M in THF, 4.3 mL) and the resulting mixture was heated to reflux. After 16 hours the mixture was cooled to 0° C. and carefully quenched with water (40 mL). After 10 minutes, the mixture was extracted three times with ethyl acetate. The combined organic extracts were dried over MgSO₄, filtered, and concentrated under reduced pressure. This residue was dissolved in CH₂Cl₂ (9 mL) and to this solution was added 4 M HCl in dioxane (15 mL) at room temp. After 4 hours, the mixture was concentrated under reduced pressure to yield the crude product, which was purified using reverse phase HPLC with acetonitrile gradient 5-40% in 30 min to yield the product. (1.95 g, 97%). H1 NMR (400 MHz, CDCl₃) δ 2.88 (t, 2H), 3.35 (t, 2H), 3.70 (t, 2H), 3.90 (t, 2H), 4.45 (s, 2H), 6.38 (d, 1H), 6.7 (d, 1H), 7.35 (m, 5H).

Step 8. Synthesis of 6-(2-bromoethyl)-1,2,3,4-tetrahydropyrido[2,3-b]pyrazine.

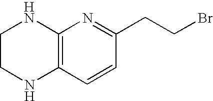

To a solution of the product of step 7 (0.4 g, 0.149 mmol) in benzene (10 mL) at room temperature under argon was added thionyl bromide (0.17 mL, 0.22 mmol) and the reaction mixture was stirred at 75° C. overnight. After cooling to room temperature, the solvent was removed in vacuo and the dark oil was purified using reverse phase HPLC with acetonitrile gradient 5-40% in 30 min to yield the product. (0.115 g, 32%) H1 NMR (400 MHz, CDCl₃) δ 3.20 (t, 2H), 3.40 (t, 2H), 3.65 (m, 4H), 6.42 (d, 2H), 6.80 (d, 2H).

Step 9. Synthesis of 3-(1,3-benzodioxol-5-yl)-4-{1-methyl-5-[2-(1,2,3,4-tetrahydropyrido[2,3-b]pyrazin-6-yl)ethoxy]-1H-pyrazol-3-yl}butanoic acid.

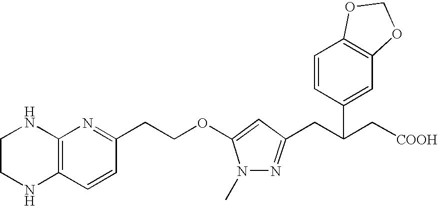

The product of step 8 and the product of Example 1, step 6, were used under conditions described in Example 16, steps 3 and 4, to give the desired product: FAB-MS:(MH⁺)=466.20. H1 NMR (500 MHz, CD₃OD) δ 2.54 (m, 1H), 2.62 (m, 2H), 2.78 (m, 1H), 3.07 (t, 2H), 3.35 (t, 2H), 3.48 (s, 3H), 3.58 (t, 2H), 4.29 (t, 2H), 5.47 (s, 1H), 5.87 (s, 2H), 6.56 (d, 1H), 6.68 (m, 3H), 6.73 (s, 1H) 6.91 (d, 1H). Anal Calcd. for C₂₄H₂₇N₅O₅ plus 2.1 CF₃COOH, 1.0H₂O: C, 46.85; H, 4.34; N, 9.69. Found: 47.02; H, 4.33; N, 9.32.

### Example 26

### 3-(1,3-benzodioxol-5-yl)-4-[5-[2-(1,2,3,4-tetrahydropyrido[2,3-b]pyrazin-6-yl)ethoxy]-1-(2,2,2-trifluoroethyl)-1H-pyrazol-3-yl]butanoic acid.

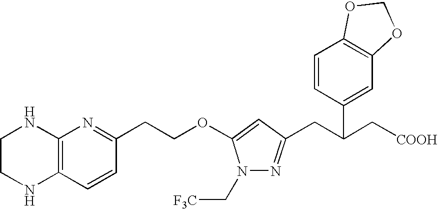

The product of step 8 and the product of Example 23, step 1, were used under conditions described in Example 16, steps 3 and 4, to give the desired product. FAB-MS:(MH⁺)=534.19. ¹H NMR (500 MHz, CD₃OD) δ 2.54 (m, 1H), 2.65 (m, 2H), 2.74 (m, 1H), 2.82 (m, 1H), 3.15 (t, 2H), 3.33 (m, 1H), 3.5 (t, 2H), 4.35 (t, 2H), 4.5 (m, 2H), 5.5 (s, 1H), 5.86 (s, 2H), 6.57 (d, 1H), 6.66 (m, 4H), 7.91 (d, 1H). Anal Calcd. for C₂₅H₂₆F₃N₅O₅ plus 1.9 CF₃COOH, 1.0H₂O: C, 45.03; H, 3.92; N, 9.12. Found: 45.56; H, 4.31; N, 8.46.

### Example 27

### 3-(1,3-benzodioxol-5-yl)-4-[5-[2-(1-methyl-1,2,3,4-tetrahydropyrido[2,3-b]pyrazin-6-yl)ethoxy]-1-(2,2,2-trifluoroethyl)-1H-pyrazol-3-yl]butanoic acid.

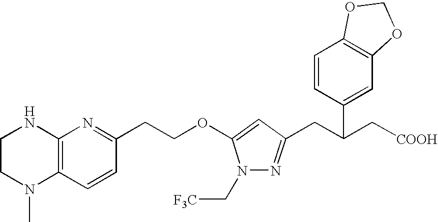

The product of Example 7, step 14, and the product of Example 23, step 1, were used under conditions described in Example 16, steps 3 and 4, to give the desired product: FAB-MS:(MH⁺)=548.21. ¹H NMR (500 MHz, CD₃OD) δ 2.54 (m, 1H), 2.64 (m, 2H), 2.74 (m, 1H), 2.82 (m, 1H), 3.4 (s, 3H), 3.19 (t, 2H), 3.31 (m, 3H), 3.65 (t, 2H), 4.30 (t, 2H), 4.5 (q, 2H), 5.48 (s, 1H), 5.87 (s, 2H), 6.65 (m, 3H), 6.71 (d, 1H), 6.91 (d, 1H). Anal Calcd. for C₂₆H₂₈F₃N₅O₅ plus 1.5 CF₃COOH, 1.5H₂O: C, 45.03; H, 3.92; N, 9.12. Found: 45.56; H, 4.31; N, 8.46

### Example 28

### 3-(1,3-benzodioxol-5-yl)-4-[5-{2-[6-(methylamino)pyridin-2-yl]ethoxy}-1-(2,2,2-trifluoroethyl)-1H-pyrazol-3-yl]butanoic acid

Step 1: Synthesis of 6-(2-bromoethyl)-N-methylpyridin-2-amine.

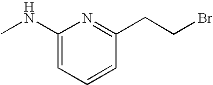

To a solution of 2-[6-(methylamino)pyridin-2-yl]ethanol (WO2002088118) and carbon tetrabromide in anhydrous dichloromethane was added slowly triphenylphosphine (3.89 g, 14.45 mmol). The reaction mixture was stirred for 1.5 hours and concentrated under reduced pressure to yield the crude mixture that was first partially purified by flash chromatography on silica gel (30% EA/Hex) to yield a yellow solid with triphenylphosphine oxide as impurity. Further purification was done by dissolving the solid obtained from flash chromatography in ethyl acetate and washing the ethyl acetate layer three times with dilute HCl. The product containing water layer was basified with 4M NaOH solution until the solution turned milky white. The mixture was extracted three times with ethyl acetate and all organic extracts were combined, dried over MgSO₄ and concentrated under reduced pressure to give the desired product (1.8 g, 64%). H1 NMR (400 MHz, CDCl₃) δ 2.75 (s, 3H), 3.12 (t, 2H), 3.60 (t, 2H), 6.25 (d, 1H), 6.45 (d, 1H), 7.35 (t, 1H).

Step 2: Synthesis of 3-(1,3-benzodioxol-5-yl)-4-[5-{2-[6-(methylamino)pyridin-2-yl]ethoxy}-1-(2,2,2-trifluoroethyl)-1H-pyrazol-3-yl]butanoic acid.

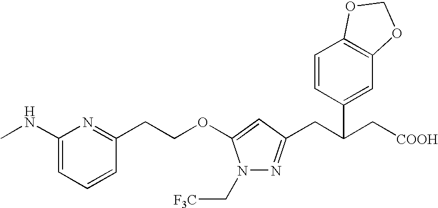

The product of step 1, and the product of Example 19, step 1, were used under conditions described in Example 16, steps 3 and 4, to give the desired product: FAB-MS:(MH⁺)=507.18. H1 NMR (500 MHz, CD₃OD) δ 2.53 (m, 1H), 2.64 (m, 2H), 2.78 (m, 2H), 3.04 (s, 3H), 3.26 (t, 2H), 3.34 (m, 1H), 4.38 (t, 2H), 4.50 (q, 2H), 5.51 (s, 1H), 5.86 (s, 2H), 6.65 (m, 2H), 6.71 (s, 1H), 6.79 (d, 1H), 6.91 (d, 1H), 7.84 (t, 1H). Anal Calcd. for C₂₄H₂₅F₃N₄O₅ plus 1.5 CF₃COOH, 0.5H₂O: C, 47.24; H, 4.04; N, 8.16. Found: 47.16; H, 4.32; N, 8.08.

### Example 29

### 3-(1,3-benzodioxol-5-yl)-4-[5-{[2-(5,6,7,8-tetrahydro-1,8-naphthyridin-2-yl)ethyl]thio}-1-(2,2,2-trifluoroethyl)-1H-pyrazol-3-yl]butanoic acid

Step 1: Synthesis of ethyl 3-(1,3-benzodioxol-5-yl)-4-[5-mercapto-1-(2,2,2-trifluoroethyl)-1H-pyrazol-3-yl]butanoate.

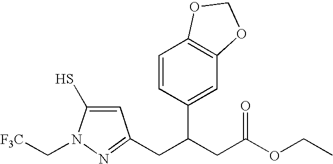

A solution of ethyl 3-(1,3-benzodioxol-5-yl)-4-[5-hydroxy-1-(2,2,2-trifluoroethyl)-1H-pyrazol-3-yl]butanoate (1.5 g, 3.75 mmol) (Example 23, step 1) and 2,4-bis(4-methoxyphenyl)-1,3,2,4-dithiadiphosphetane 2,4-disulfide (0.91 g, 2.25 mmol) in benzene (12 mL) was stirred at 60° C. overnight. The reaction mixture was purified by flash chromatography on silica gel (10% EA/Hex) to yield the desired product (1.45 g, 93%) as a yellow oil. H1 NMR (500 MHz, CDCl₃) δ 1.05 (t, 3H), 2.50 (m, 1H), 2.58 (m, 1H), 2.80 (t, 2H), 2.80 (t, 2H), 3.30 (m, 1H), 3.95 (q, 2H) 4.72 (q, 2H), 5.84 (s, 2H), 6.18 (s, 1H), 6.60 (m, 3H).

Step 2: Synthesis of 3-(1,3-benzodioxol-5-yl)-4-[5-{[2-(5,6,7,8-tetrahydro-1,8-naphthyridin-2-yl)ethyl]thio}-1-(2,2,2-trifluoroethyl)-1H-pyrazol-3-yl]butanoic acid.

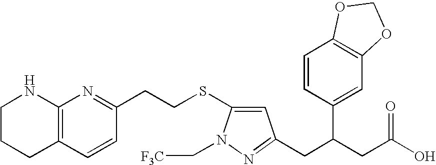

The product was obtained using the product of step 1 under the conditions described in Example 3, steps 2 and 3. FAB-MS:(MH⁺)=549. H1 NMR (500 MHz, CD₃OD) δ 1.95 (m, 2H), 2.60 (m, 1H), 2.70 (m, 1H), 2.82 (t, 2H), 2.90 (m, 1H), 3.20 (m, 3H), 3.40 (m, 1H) 3.5 (t, 2H), 3.7 (t, 2H), 5.20 (q, 2H), 5.82 (d, 2H), 6.58 (d, 1H), 6.3 (m, 3H), 7.55 (d, 1H). Anal Calcd. for C₂₆H₂₇F₃N₄O₆S plus 1.3 CF₃COOH, 2.6H₂O: C, 44.29; H, 4.35; N, 722. Found: 43.88; H, 4.75; N, 7.74.

### Example 30

### 3-(1,3-benzodioxol-5-yl)-4-[5-{[2-(5,6,7,8-tetrahydro-1,8-naphthyridin-2-yl)ethyl]sulfonyl}-1-(2,2,2-trifluoroethyl)-1H-pyrazol-3-yl]butanoic acid.

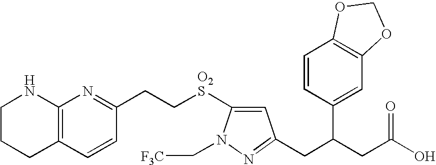

Using the procedure described in Example 4, steps 1 and 2, ethyl 3-(1,3-benzodioxol-5-yl)-4-[5-{[2-(5,6,7,8-tetrahydro-1,8-naphthyridin-2-yl)ethyl]thio}-1-(2,2,2-trifluoroethyl)-1H-pyrazol-3-yl]butanoate gave the desired product. (0.180 g). FAB-MS:(MH⁺)=581.3. H1 NMR (500 MHz, CD₃OD) δ 1.95 (m, 2H), 2.57 (m, 1H), 2.67 (m, 1H), 2.84 (m, 2H), 2.94 (m, 1H), 3.10 (m, 3H), 3.35 (m, 1H) 3.50 (t, 2H), 5.83 (m, 2H), 6.18 (s, 1H), 6.54 (d, 1H), 6.35 (m, 2H), 6.69 (s, 1H), 7.56 (d, 1H). Anal Calcd. for C₂₆H₂₇F₃N₄O₄S plus 1.4 CF₃COOH, 0.5H₂O: C, 44.29; H, 4.35; N, 722. Found: 43.88; H, 4.75; N, 7.74.

### Example 31

### 3-(1,3-benzodioxol-5-yl)-4-{1-methyl-5-[2-(1-methyl-1,2,3,4-tetrahydropyrido[2,3-b]pyrazin-6-yl)ethoxy]-1H-pyrazol-3-yl}butanoic acid.

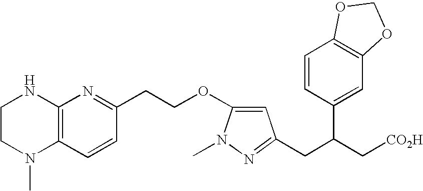

The title compound was obtained using the procedure described in Example 7, Steps 15 and 16 but using the product of Example 1, step 6 in place of the product of Example 7, Step 6. The desired product was obtained as an yellow oil. FAB-MS:(MH⁺)=480. ¹H NMR (500 MHz, CD₃OD) δ 2.61 (m, 2H), 2.80 (m, 2H), 2.91 (s, 3H), 3.08 (t, 2H), 3.30 (m, 3H), 3.48 (s, 3H), 3.65 (t, 2H), 4.31 (t, 2H), 5.52 (s, 1H), 5.85 (s, 2H), 6.69 (m, 4H), 6.89 (d, 1H). Anal Calcd. for C₂₅H₂₉N₅O₅ plus 2.2 CF₃COOH: C, 48.44; H, 4.21; N, 9.69. Found: 48.35; H, 4.31; N, 9.59.

### Example 32

### 3-(1,3-Benzodioxol-5-yl)-4-{1-methyl-5-[2-(4-methyl-5,6,7,8-tetrahydro-1,8-naphthyridin-2-yl)ethoxy]-1H-pyrazol-3-yl}butanoic acid.

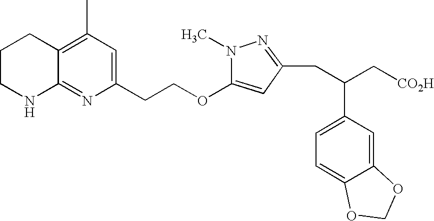

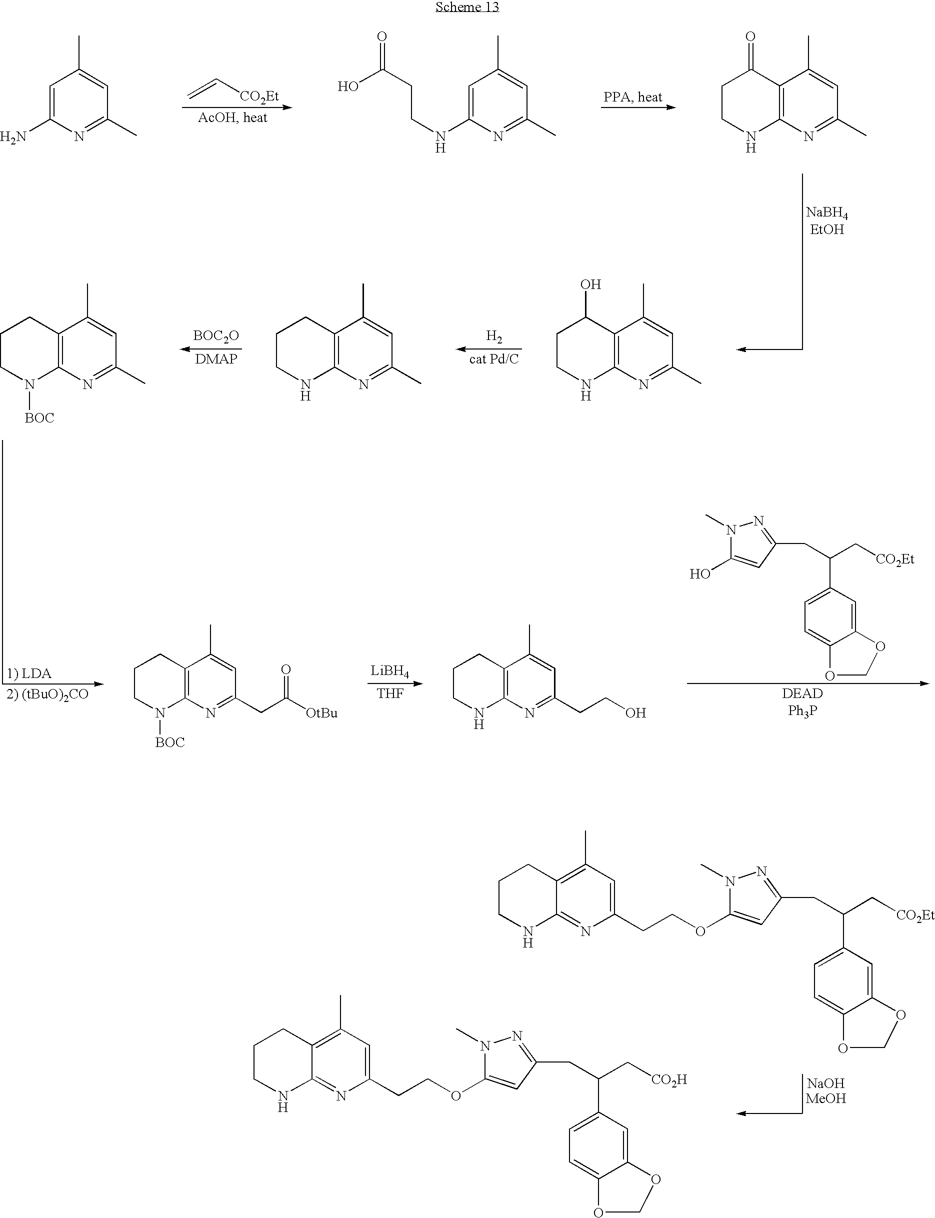

Step 1. Synthesis of ethyl N-(2,4-dimethylpyrid-5-yl)alanine.

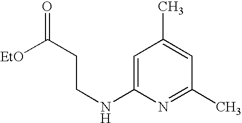

Commercially available 2-amino-4,6-dimethylpyridine (25.46 g) was combined with 26 mL of ethyl acrylate and to this solution was added 6.0 mL of glacial acetic acid. The solution was heated to 130° C. under an atmosphere of argon for 3 days. The reaction was cooled and then 116 mL of 6N aqueous sodium hydroxide was added and the reaction was heated to 100° C. for 40 minutes. After the reaction was cooled, the pH was adjusted to 5 with concentrated hydrochloric acid. The precipitate was collected and washed with fresh water and then hexane. The mother liquors were washed with ethyl acetate or methylene chloride to give 33 g of product. ¹H NMR, 300 MHz, CD₃OD δ 6.92 (1H, s); 6.81 (1H, s); 4.10 (2H, t, J=7 Hz); 3.13 (2H, t, J=7 Hz); 2.95 (3H, s); 2.83 (3H, s).

Step 2. Synthesis of 5,7-dimethyl-2,3-dihydro-1,8-naphthyridin-4(1H)-one.

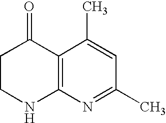

The product (5 g) from step 1 was suspended in 75 g of polyphosphoric acid (PPA) and heated for 40 minutes at 120° C. The reaction mixture was transferred to a glass beaker to cool down and then added portion-wise to ice then stirring till the viscous oil completely dissolved. The solution was kept at 0° C. at all times. The pH of the resulting solution was adjusted between 8 and 9 with cold concentrated ammonium hydroxide. The resulting solid was filtered from the solution then washed with water then dissolved in methylene chloride. This solution was washed with brine, dried (MgSO₄) and the solvent was removed under reduced pressure. The resulting solid was dried under high vacuum and then washed with absolute ethanol to give the desired product (2.89 g) whose purity was acceptable for use in the next step. ¹H NMR, 400 MHz, CDCl₃δ 6.34 (1H, s); 5.58 (1H, br. s); 3.57 (2H, m); 2.68 (2H, t, J=7 Hz); 2.56 (3H, s); 2.33 (3H, s).

Step 3. Synthesis of 5,7-dimethyl-1,2,3,4-tetrahydro-1,8-naphthyridin-4-ol.

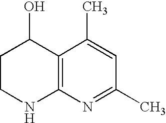

The ketone from step 2 was added portion wise to a solution of sodium borohydride (259.89 mg; 6.87 mmol) in 11 mL of ethanol. Starting material was still observed by TLC, thus an additional 250 mg of sodium borohydride was added and stirred till most the starting material had disappeared by TLC. The reaction mixture stood over 2 days exposed to air in which a solid was observed to form in the flask. The reaction mixture was diluted with methylene chloride and washed with water. The pH of aqueous layer was adjusted to pH of 7 by adding 1N aqueous hydrochloric acid. This solution was extracted with methylene chloride and the organic extracts were dried, filtered and evaporated under reduced pressure to give the product as foam. The product was taken on to the next step without further purification. ¹H NMR, 400 MHz, CDCl₃ δ 6.22 (1H, s); 5.30 (1H, br. s); 4.83 (1H, t, J=2.5 Hz); 3.83 (1H, br. s); 3.39 (1H, ddd, J=13, 12, 3 Hz); 3.20 (1H, br. d, J=13 Hz); 2.24 (3H, s); 2.20 (3H, s); 1.96 (1H, dq, J=13, 2.5 Hz); 1.70 (1H, tdd, J=13, 5, 3 Hz).

Step 4. Synthesis of 5,7-dimethyl-1,2,3,4-tetrahydro-1,8-naphthyridine.

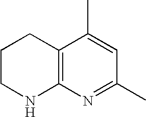

The compound from step 3 was submitted to catalytic (Pd/C) hydrogenation conditions and the product isolated as the acetic acid salt. This was converted to the neutral amine by treating with an excess of concentrated ammonium hydroxide followed by lyophilzation. ¹H NMR, 400 MHz, CDCl₃δ 7.82 (1H, br. s); 6.23 (1H, s); 3.40 (2H, t, J=6 Hz); 2.61 (2H, t, J=7 Hz); 2.39 (3H, s); 2.17 (3H, s); 1.92 (2H, p, J=6 Hz).

Step 5. Synthesis of tert-butyl 5,7-dimethyl-3,4-dihydro-1,8-naphthyridine-1 (2H)-carboxylate.

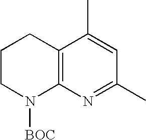

520 mg of the product of step 4 was dissolved in 6.4 mL of tetrahydrofuran and to this solution was added 1.0 g of BOC anhydride followed by 23.5 mg of dimethylaminopyridine. The reaction was heated to 50° C. overnight. The following day the solvent was found to evaporated from the reaction. The crude compound was purified by flash column (SiO₂) eluting first with 100% hexane followed by 50% EA/hexane. The desired product was isolated in 83% yield (696 mg). ¹H NMR, 400 MHz, CDCl₃δ 6.72 (1H, s); 3.73 (2H, m); 2.63 (2H, t, J=7 Hz); 2.43 (3H, s); 2.18 (3H, s); 1.93 (2H, m); 1.51 (9H, s).

Step 6. Synthesis of tert-butyl 7-(2-tert-butoxy-2-oxoethyl)-5-methyl-3,4-dihydro-1,8-naphthyridine-1 (2H)-carboxylate.

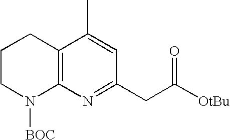

Lithium diisopropylamine (1.59 mmol; 3.19 mmol) was added to a solution of 697 mg the product from step 5 dissolved in a solution of 10 mL of dry THF at −78° C. After 20 minutes, a 1M solution of di-t-butylcarbonate (9.8 mL; 9.8 mmol) was added. After 1 hr, the reaction was quenched with a saturated solution of ammonium chloride and warmed to 25° C. The mixture was extracted three times with ethyl acetate. The combined organic extracts were washed with brine, dried (MgSO4), filtered and filtrates were concentrated under reduced pressure. The crude product was purified by chromatography (SiO2, 25% ethyl acetate/ Hexane) to give 612 mg of the desired product.

Step 7. Synthesis of 2-(4-methyl-5,6,7,8-tetrahydro-1,8-naphthyridin-2-yl)ethanol.

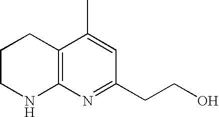

The product from step 6 (612 mg; 1.69 mmol) was dissolved in 7.7 mL of THF at 25° C. and to this solution was added a solution of lithium borohydride (2.0 M in THF). After 12 hr, the mixture was cooled to 0° C. and then carefully quenched with 6 mL of water. After stirring for 10 minutes, the mixture was extracted three times with ethyl acetate. The combined organic extracts were washed with brine, dried (MgSO4), filtered and concentrated under reduced pressure to give a solid, which was taken directly to the next step without further purification. The crude product from was dissolved in 4 M HCl dioxane at 25° C. over night. The reaction mixture was concentrated under reduced pressure. The crude residue was chromatographed (SiO₂, 94.5/5/0.5 methylene chloride/ethanol/ concentrated ammonium hydroxide to give 190 mg of the desired product. ¹H NMR, 400 MHz, CD₃OD δ 6.31 (1H, s); 3.79 (2H, t, J=7 Hz); 3.32 (2H, app. t, J=6 Hz); 2.70 (2H, t, J=7 Hz); 2.61 (2H, t, J=6.5 Hz); 2.12 (3H, s); 1.90 (2H, p, J=6 Hz).

Step 8. Synthesis of ethyl 3-(1,3-benzodioxol-5-yl)-4-{1-methyl-5-[2-(4-methyl-5,6,7,8-tetrahydro-1,8-naphthyridin-2-yl)ethoxy]-1H-pyrazol-3-yl}butanoate.

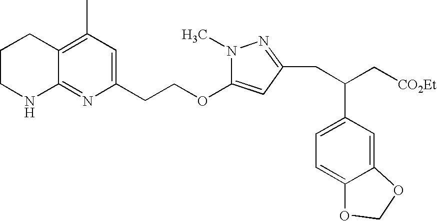

The title compound was obtained using the procedure described in Example 1, step 7, using the product of step 7 above and the product of Example 1, step 6. ¹H NMR, 400 MHz, CD₃OD δ 6.73 (1H, d, J=1 Hz); 6.70 (1H, d, J=8 Hz); 6.67 (1H, dd, J=8, 1 Hz); 6.31 (1H, s); 5.88 (1H, d, J=1 Hz); 5.87 (1H, d, J=1 Hz); 5.37 (1H, s); 4.25 (2H, t, J=6.5 Hz); 3.98 (2H, q, J=7 Hz); 3.33 (3H, m); 2.90 (2H, t, J=6.5 Hz); 2.74 (2H, d, J=7 Hz); 2.66 (1H, dd, J=15, 6 Hz); 2.62 (2H, t, J=7 Hz); 2.54 (1H, dd, J=15, 10 Hz); 2.12 (3H, s); 1.90 (2H, p, J=6 Hz); 1.11 (3H, t, J=7 Hz).

Step 9. Synthesis of 3-(1,3-benzodioxol-5-yl)-4-{1-methyl-5-[2-(4-methyl-5,6,7,8-tetrahydro-1,8-naphthyridin-2-yl)ethoxy]-1H-pyrazol-3-yl}butanoic acid.

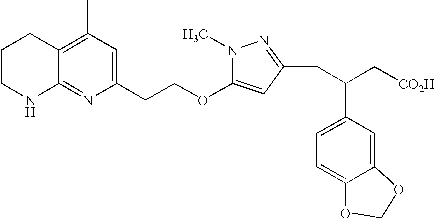

80 mg of the impure mixture obtained from step 8 was dissolved in 5 mL of methanol. To this solution was added 5 mL of 1N aqueous sodium hydroxide. The reaction was stirred overnight at 25° C. The following day the pH of the reaction mixture was adjusted to 2 then stripped to dryness. The crude mixture was purified by reverse phase HPLC to give 35 mg of the desired product. 400 MHz Proton NMR, CD₃OD δ 6.73 (1H, d, J=1 Hz), 6.69 (1H, d, J=8 Hz), 6.67 (1H, dd, J=8, 1 Hz), 6.60 (1H, s), 5.87(2H, s), 5.57(1H, s), 4.36 (2H, t, J=6 Hz), 3.49 (3H, s), 3.45 (2H, t, J=6 Hz), 3.33 (1H, p, J=7.5 Hz), 3.12 (2H, t, J=6 Hz), 2.84 (1H, dd, J=14, 7 Hz), 2.77(1H, dd, J=14, 8 Hz), 2.73 (2H, t, J=6 Hz), 2.64(1H, dd, J=15, 6 Hz), 2.53 (1H, dd, J=15, 9 Hz), 2.29(3H, s), 1.97, 2H, p, J=6 Hz. Anal. Calcd. for C₂₆H₃₀N₄O₅ plus 1.9 CF₃CO₂H and 1.3H₂O: C, 49.81; H, 4.84; N, 7.80. Found: C, 49.27; H, 4.76; N, 8.49.

Select examples of α_{V}β₃ and/or α_{V}β₅ integrin antagonists are depicted in Table 1 below along with their corresponding plasma level upon oral dosing (AUC-PO) level.

TABLE 1

| | | | Rat AUC- |
|---|---|---|---|
| | | | PO/dose |
| Example | | | (ug*hr/mL/ |
| No. | Structure | Compound Name | mg/kg) |
| 1 | 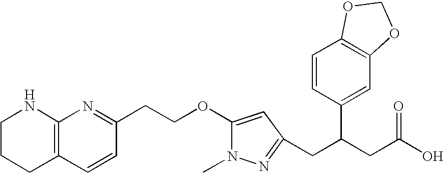 | 3-(1,3-benzodioxol-5- yl)-4-{1-methyl-5-[2- (5,6,7,8-tetrahydro- 1,8-naphthyridin-2- yl)ethoxy]-1H-pyrazol- 3-yl}butanoic acid | 0.9 |
| 21 | 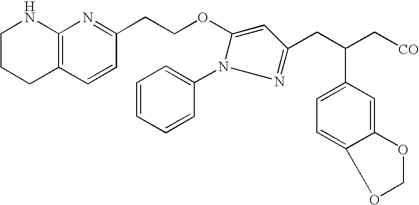 | 3-(1,3-benzodioxol-5- yl)-4-{1-phenyl-5-[2- (5,6,7,8-tetrahydro- 1,8-naphthyridin-2- yl)ethoxy]-1H-pyrazol- 3-yl}butanoic acid | 1.7 |
| 31 | 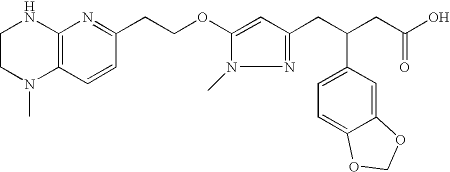 | 3-(1,3-benzodioxol-5- yl)-4-{1-methyl-5-[2- (1-methyl-1,2,3,4- tetrahydropyrido[2,3- b]pyrazin-6-yl)ethoxy]- 1H-pyrazol-3- yl}butanoic acid | 1.9 |
| 32 | 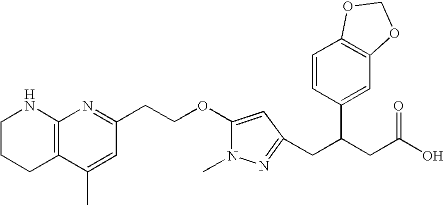 | 3-(1,3-benzodioxol-5- yl)-4-{1-methyl-5-[2- (4-methyl-5,6,7,8- tetrahydro-1,8- naphthyridin-2- yl)ethoxy]-1H-pyrazol 3-yl}butanoic acid | 2.2 |
| 2 | 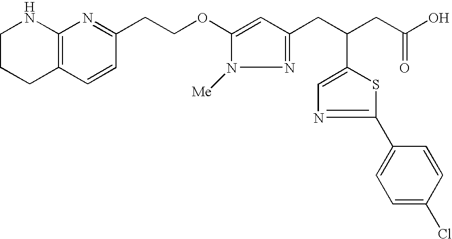 | 3-[2-(4-chlorophenyl)- 1,3-thiazol-5-yl]-4-{1- methyl-5-[2-(5,6,7,8- tetrahydro-1,8- naphthyridin-2- yl)ethoxy]-1H-pyrazol- 3-yl}butanoic acid | 1 |
| 19 | 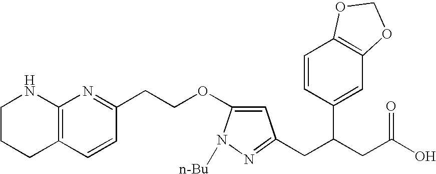 | 3-(1,3-benzodioxol-5- yl)-4-{1-butyl-5-[2- (5 | |
| 12 | 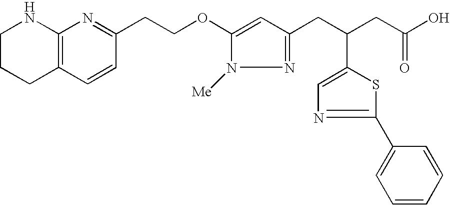 | 4-{1-methyl-5-[2- (5,6,7,8-tetrahydro- 1,8-naphthyridin-2- yl)ethoxy]-1H-pyrazol- 3-yl}-3-(2-phenyl-1,3- thiazol-5-yl)butanoic acid | |
| 8 | 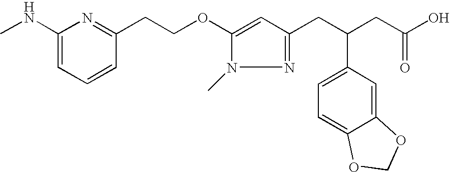 | 3-(1,3-benzodioxol-5- yl)-4-(1-methyl-5-{2- [6- (methylamino)pyridin- 2-yl]ethoxy}-1H- pyrazol-3-yl)butanoic acid | 0.7 |
| 16 | 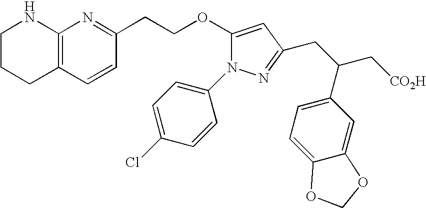 | 3-(1,3-benzodioxol-5- yl)-4-{1-(4- chlorophenyl)-5-[2- (5,6,7,8-tetrahydro- 1,8-naphthyridin-2- yl)ethoxy]-1H-pyrazol- 3-yl}butanoic acid | |
| 3 | 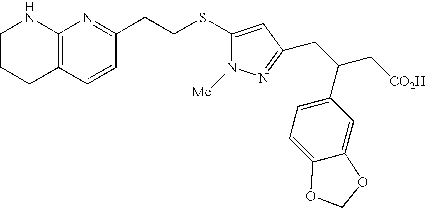 | 3-(1,3-benzodioxol-5- yl)-4-(1-methyl-5-{[2- (5,6,7,8-tetrahydro- 1,8-naphthyridin-2- yl)ethyl]thio}-1H- pyrazol-3-yl)butanoic acid | 2.2 |
| 4 | 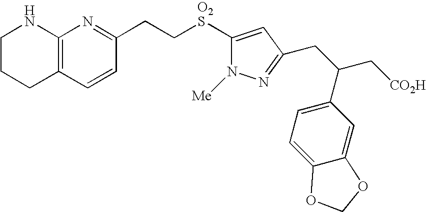 | 3-(1,3-benzodioxol-5- yl)-4-(1-methyl-5-{[2- (5,6,7,8-tetrahydro- 1,8-naphthyridin-2- yl)ethyl]sulfonyl}-1H- pyrazol-3-yl)butanoic acid | 0.7 |
| 26 | 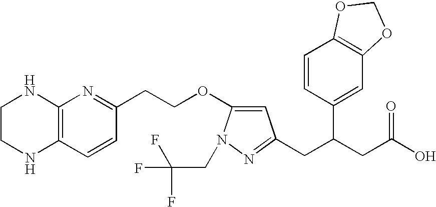 | 3-(1,3-benzodioxol-5- yl)-4-[5-[2-(1,2,3,4- tetrahydropyrido[2,3- b]pyrazin-6-yl)ethoxy]- 1-(2,2,2-trifluoroethyl)- 1H-pyrazol-3- yl]butanoic acid | 0.9 |
| 5 | 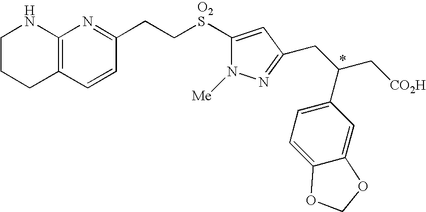 | (S)-3-(1,3- benzodioxol-5-yl)-4- {1-methyl-5-[2- (5,6,7,8-tetrahydro- 1,8-naphthyridin-2- yl)ethoxy]-1H-pyrazol- 3-yl}butanoic acid | 0.6 |
| 25 | 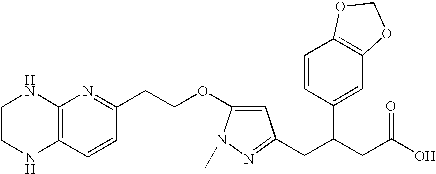 | 3-(1,3-benzodioxol-5- yl)-4-{1-methyl-5-[2- (1,2,3,4- tetrahydropyrido[2,3- b]pyrazin-6-yl)ethoxy]- 1H-pyrazol-3- yl}butanoic acid | 0.4 |
| 27 | 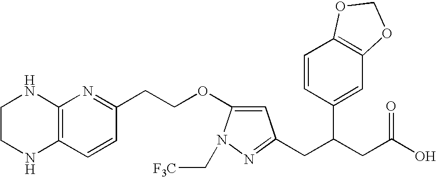 | 3-(1,3-benzodioxol-5- yl)-4-[5-[2-(1-methyl- 1,2,3,4- tetrahydropyrido[2,3- b]pyrazin-6-yl)ethoxy]- 1-(2,2,2-trifluoroethyl)- 1H-pyrazol-3- yl]butanoic acid | 2 |
| 17 | 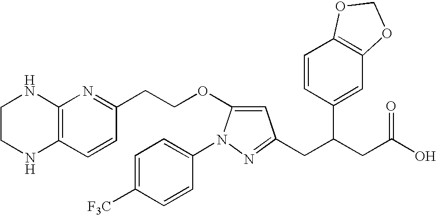 | 3-(1,3-benzodioxol-5- yl)-4-{5-[2-(5,6,7,8- tetrahydro-1,8- naphthyridin-2- yl)ethoxy]-1-[4- (trifluoromethyl)phenyl]- 1H-pyrazol-3- yl}butanoic acid | |
| 9 | 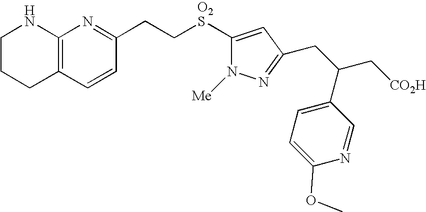 | 3-(6-methoxypyridin- 3-yl)-4-{1-methyl-5-[2- (5,6,7,8-tetrahydro- 1,8-naphthyridin-2- yl)ethoxy]-1H-pyrazol- 3-yl}butanoic acid | 1 |
| 14 | 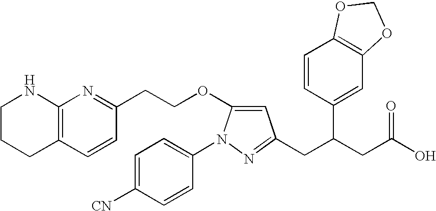 | 3-(1,3-benzodioxol-5- yl)-4-{1-(4- cyanophenyl)-5-[2- (5,6,7,8-tetrahydro- 1,8-naphthyridin-2- yl)ethoxy]-1H-pyrazol- 3-yl}butanoic acid | |
| 28 | 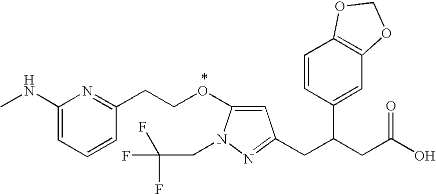 | 3-(1,3-benzodioxol-5- yl)-4-[5-{2-[6- (methylamino)pyridin- 2-yl]ethoxy}-1-(2,2,2- trifluoroethyl)-1H- pyrazol-3-yl]butanoic acid | |
| 23 | 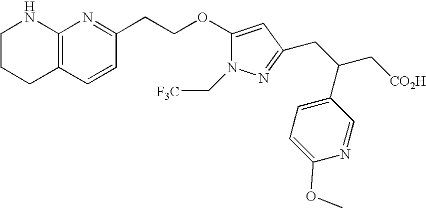 | 3-(6-methoxypyridin- 3-yl)-4-[5-[2-(5,6,7,8- tetrahydro-1,8- naphthyridin-2- yl)ethoxy]-1-(2,2,2- trifluoroethyl)-1H- pyrazol-3-yl]butanoic acid | 0.8 |
| 7 | 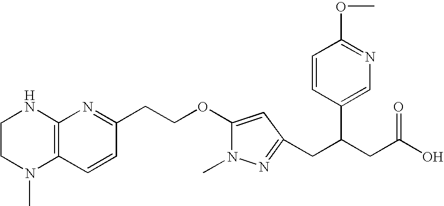 | 3-(6-methoxypyridin- 3-yl)-4-{1-methyl-5-[2- (1-methyl-1,2,3,4- tetrahydropyrido[2,3- b]pyrazin-6-yl)ethoxy]- 1H-pyrazol-3- yl}butanoic acid | 0.8 |
| 6 | 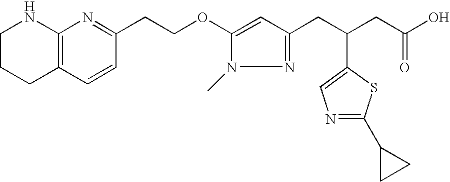 | 3-(2-cyclopropyl-1,3- thiazol-5-yl)-4-{1- methyl-5-[2-(5,6,7,8- tetrahydro-1,8- naphthyridin-2- yl)ethoxy]-1H-pyrazol- 3-yl}butanoic acid | 1.1 |
| 15 | 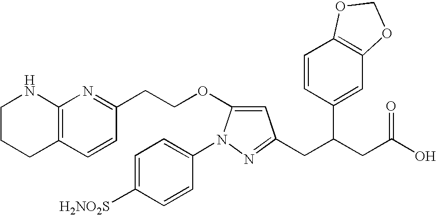 | 4-{1-[4- (aminosulfonyl)phenyl]- 5-[2-(5,6,7,8- tetrahydro-1,8- naphthyridin-2- yl)ethoxy]-1H-pyrazol- 3-yl}-3-(1,3- benzodioxol-5- yl)butanoic acid | |
| 13 | 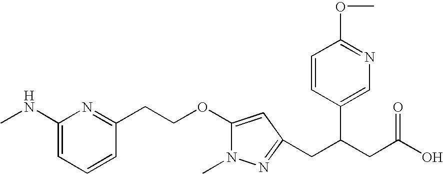 | 3-(6-methoxypyridin- 3-yl)-4-(1-methyl-5-{2- [6- (methylamino)pyridin 2-yl]ethoxy}-1H- pyrazol-3-yl)butanoic acid | |
| 29 | 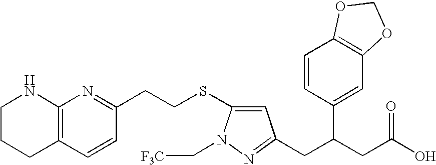 | 3-(1,3-benzodioxol-5- yl)-4-[5-{[2-(5,6,7,8- tetrahydro-1,8- naphthyridin-2- yl)ethyl]thio}-1-(2,2,2- trifluoroethyl)-1H- pyrazol-3-yl]butanoic acid | |
| 10 | 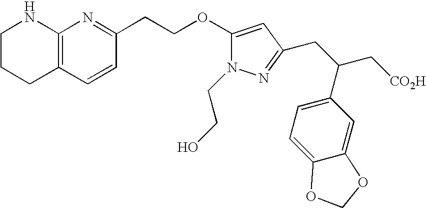 | 3-(1,3-benzodioxol-5- yl)-4-{1-(2- hydroxyethyl)-5-[2- (5,6,7,8-tetrahydro- 1,8-naphthyridin-2- yl)ethoxy]-1H-pyrazol- 3-yl}butanoic acid | |
| 30 | 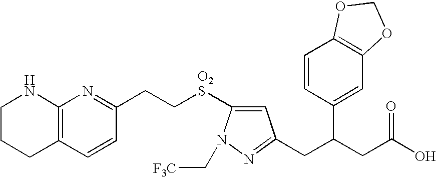 | 3-(1,3-benzodioxol-5- yl)-4-[5-{[2-(5,6,7,8- tetrahydro-1,8- naphthyridin-2- yl)ethyl]sulfonyl}-1- (2,2,2-trifluoroethyl)- 1H-pyrazol-3- yl]butanoic acid | |
| 24 | 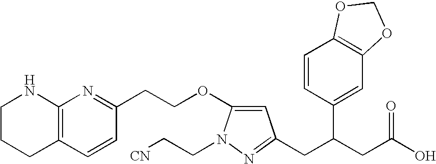 | 4-{1-(2-cyanoethyl)-5- [2-(5,6,7,8-tetrahydro- 1,8-naphthyridin-2- yl)ethoxy]-1H-pyrazol- 3-yl}-3-(6- methoxypyridin-3- yl)butanoic acid | |
| 22 | 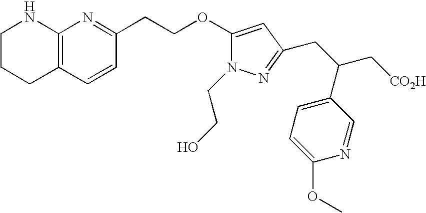 | 4-{1-(2-hydroxyethel)- 5-[2-(5,6,7,8- tetrahydro-1,8- naphthyridin-2- yl)ethoxy]-1H-pyrazol- 3-yl}-3-(6- methoxypyridin-3- yl)butanoic acid | |
| 11 | 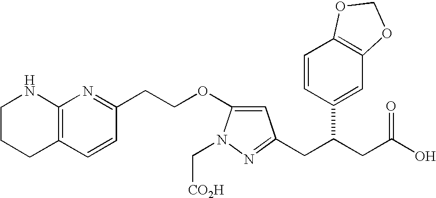 | (S)-3-(1,3- benzodioxol-5-yl)-4- {1-(carboxymethyl)-5- [2-(5,6,7,8-tetrahydro- 1,8-naphthyridin-2- yl)ethoxy]-1H-pyrazol- 3-yl}butanoic acid | |

1. A compound corresponding to Formula I 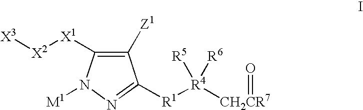
or a pharmaceutically acceptable salt thereof,
wherein:
M¹ is selected from the group consisting of heteroaryl, acyl, and optionally substituted hydrocarbyl, wherein the optional substituents are selected from the group consisting of alkyl, halo, haloalkyl, hydroxy, alkoxy, amino, alkylamino, dialkylamino, cyano, acyl, —S—, —SO—, —SO₂—, sulfonamido, aryl, and heteroaryl;
R¹ is selected from the group consisting of —CH(R²)—, —N(R³)—, —O—, —S—, —so—, —S(O)₂—, —NHS(O)₂—, —S(O)₂NH— and —C(O)—;
R² is selected from the group consisting of hydrogen, hydroxy, and optionally substituted hydrocarbyl or alkoxy, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl, or R² in combination with R⁷ forms a lactone;
R³ is selected from the group consisting of hydrogen and optionally substituted hydrocarbyl, heteroaryl, and acyl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
R⁴ is carbon or nitrogen;
R⁵ is selected from the group consisting of hydrogen, halo, optionally substituted hydrocarbyl, and heteroaryl, wherein the optional substituents are selected from the group consisting of alkyl, halogen, hydroxy, alkoxy, alkoxyalkyl, amino, alkylamino, dialkylamino, cyano, acyl, —S—, —SO—, —SO₂—, sulfonamido, heteroaryl, and optionally substituted aryl, wherein the optional substituent is halo, or R⁵ together with R⁴ and R⁶ forms a monocyclic or bicyclic ring system;
R⁶ is an electron pair when R⁴ is nitrogen, or R⁶ is hydrogen, halo, or optionally substituted hydrocarbyl, or heterocyclo when R⁴ is carbon, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroary, or R⁶ together with R⁴ and R⁵ forms a monocyclic or bicyclic ring system;
R⁷ is selected from the group consisting of —OR⁸, —SR⁸, and —NR⁸R⁹, or R⁷ in combination with R² forms a lactone;
R⁸ is selected from the group consisting of hydrogen and optionally substituted hydrocarbyl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
R⁹ is selected from the group consisting of hydrogen, hydroxy, and optionally substituted hydrocarbyl or alkoxy, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
X¹ is selected from the group consisting of a bond —O—, —CH₂—, —CH₂O—, —NH—, —C(O)—, —S—, —S(O)—CH(OH)—, —S(O)₂—, alkenyl, and alkynyl;
X² is a linker comprising a chain of 1 to 6 atoms, optionally substituted, optionally unsaturated, selected from the group consisting of C, O, S and N;
X³ is heterocyclic; and
Z¹ is selected from the group consisting of hydrogen, hydroxy, cyano, and optionally substituted hydrocarbyl or heteroaryl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl.

2. The compound or salt of claim 1 wherein:
M¹ is selected from the group consisting of heteroaryl; —(CH₂)ₘCN wherein m is 1-4; —(CH₂)ₘCOM² wherein m is 1-4 and M² is selected from the group consisting of hydroxy, alkoxy, alkyl, amino, alkylamino, dialkylamino, and arylamino; and optionally substituted alkyl or aryl, wherein the optional substituents are selected from the group consisting of alkyl, halogen, haloalkyl, hydroxy, alkoxy, amino, alkylamino, dialkylamino, cyano, acyl, —S—, —SO—, —SO₂—, sulfonamido, aryl, and heteroaryl;
Z¹ is selected from the group consisting of hydrogen, and optionally substituted alkyl, heteroaryl, or aryl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl; and
X² is a carbon chain comprising 1 to 3 carbon atoms with or without a carbon-carbon unsaturated bond.

3. The compound or salt of claim 1 wherein:
X¹ is —O—, —S—, —SO—, —SO₂—, —N—, or —CH₂—;
R² is H, hydroxy, or alkoxy;
R³ is hydrogen;
R⁴ is carbon or nitrogen;
R⁵ is selected from the group consisting of hydrogen and optionally substituted alkyl, heteroaryl, or aryl, wherein the optional substituents are selected from the group consisting of alkyl, halogen, hydroxy, alkoxy, alkoxyalkyl, amino, alkylamino, dialkylamino, cyano, acyl, —S—, —SO—, —SO₂—, sulfonamido, aryl, and heteroaryl;
R⁶ is selected from the group consisting of hydrogen, an electron pair, and optionally substituted alkyl, heteroaryl, or aryl, wherein the optional substituents are selected from the group consisting of alkyl, halogen, hydroxy, alkoxy, amino, alkylamino, dialkylamino, cyano, acyl, —S—, —SO—, —SO₂, sulfonamido, aryl, and heteroaryl;
R⁷ is hydroxy or alkoxy;
X³ is selected from the group consisting of: 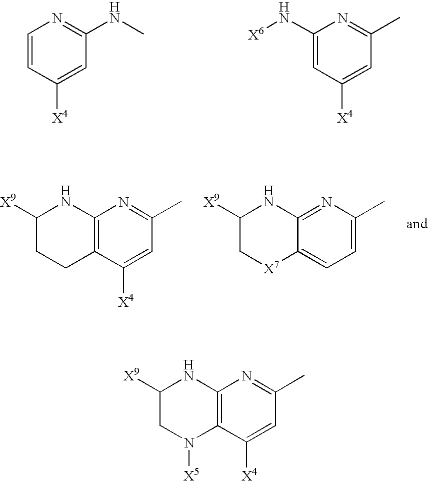
wherein:
X⁴ is selected from the group consisting of hydrogen, hydroxy, alkoxy, amino, alkylamino, dialkylamino, cyclicamino, heteroaryl, —N—SO₂Rx wherein Rx is alkyl or aryl, and optionally substituted hydrocarbyl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
X⁵, X⁶, and X⁸ are independently selected from the group consisting of hydrogen and optionally substituted hydrocarbyl or heteroaryl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
X⁷ is selected from the group consisting of —CH₂—, —CH₂O—, —OCH₂—, —S—, —SO—, —SO₂—, —O—, —C(O)—, —CH(OH)—, —NH—, and —NX⁸; and
X⁹ is ═O, or —OH.

4. The compound or salt of claim 1 wherein R⁴, R⁵, and R⁶ form a monocyclic or bicyclic ring.

5. The compound or salt of claim 1 wherein the compound has the structure: 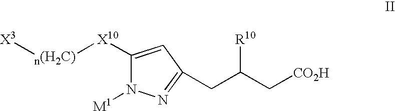
wherein:
M¹ is selected from the group consisting of phenyl, methyl, hydroxyethyl, carboxymethyl, trifluoroethyl, and cyanoethyl;
n is 1-3;
R¹⁰ is monocyclic or bicyclic aryl, aralkyl, heteroaralkyl, or heteroaryl optionally containing 1-5 heteroatoms, all optionally substituted;
X³ is selected from the group consisting of: 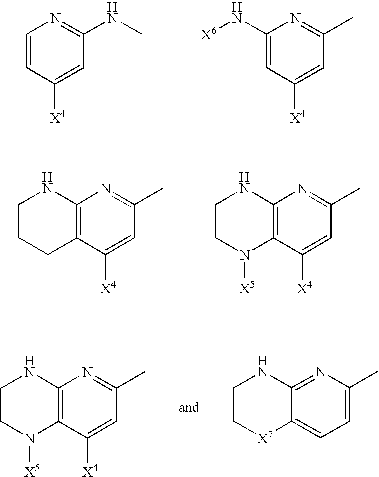
X⁴ is hydrogen, hydroxy, and optionally substituted hydrocarbyl, alkoxy, amino, or heteroaryl, wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl;
X⁵, X⁶, and X⁸ are independently hydrogen, or optionally substituted hydrocarbyl or heteroaryl wherein the optional substituents are selected from the group consisting of alkyl, halogen, hydroxy, alkoxy, amino, alkylamino, dialkylamino, cyano, acyl, —S—, —SO—, —SO₂—, sulfonamido, aryl, and heteroaryl; and
X⁷ is —CH₂—, —CH₂O—, —OCH₂— —S—, —O—, —C(O)—, —CH(OH)—, —NH—, or —NX⁸.

6. The compound or salt of claim 5 wherein R¹⁰ is optionally substituted by one or more substituents selected from the group consisting of alkyl, haloalkyl, aryl, heteroaryl, halogen, alkoxyalkyl, aminoalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)ₘCOR wherein m is 0-2 and R is hydroxy, alkoxy, alkyl or amino.

7. The compound or salt of claim 6 wherein the compound is the “S” isomer.

8. The compound or salt of claim 1 wherein the compound or a pharmaceutically acceptable salt is selected from the group consisting of: 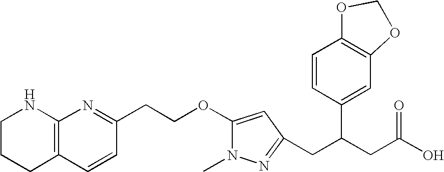
a) 3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoic acid; 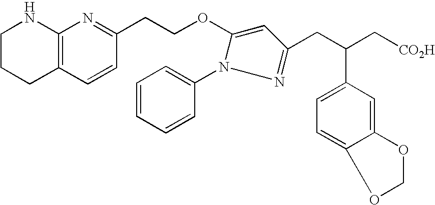
b) 3-(1,3-benzodioxol-5-yl)-4-{1-phenyl-5-[2-(5,6,7,8-tetrahydro-1,8-naphthyridin-2-yl)ethoxy]-1H-pyrazol-3-yl}butanoic acid; 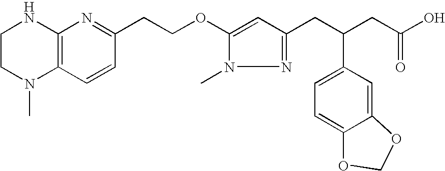
c) 3-(1,3-benzodioxol-5-yl)-4-{1-methyl-5-[2-(1-methyl-1,2,3,4-tetrahydropyrido[2,3-b]pyrazin-6-yl)ethoxy]-1H-pyrazol-3-yl}butanoic acid; 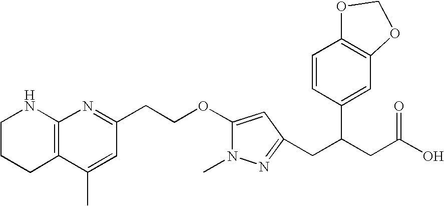
d) 33-(1,3-benzodioxol-5-yl)-4-{1-methyl-5-[2-(4-methyl-5,6,7,8-tetrahydro-1,8-naphthyridin-2-yl)ethoxy]-1H-pyrazol-3-yl}butanoic acid; 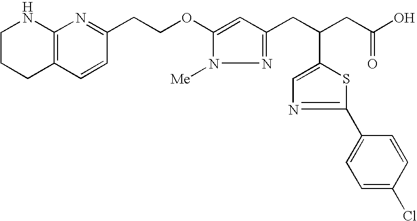
e) 3-[2-(4-chlorophenyl)-1,3-thiazol-5-yl]-4-{1-methyl-5-[2-(5,6,7,8-tetrahydro-1,8-naphthyridin-2-yl)ethoxy]-1H-pyrazol-3-yl}butanoic acid; 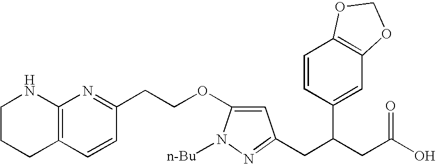
f) 3-(1,3-benzodioxol-5-yl)-4-{1-butyl-5-[2-(5,6,7,8-tetrahydro-1,8-naphthyridin-2-yl)ethoxy]-1H-pyrazol-3-yl}butanoic acid; 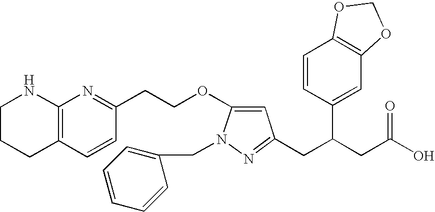
g) 3-(1,3-benzodioxol-5-yl)-4-{1-benzyl-5-[2-(5,6,7,8-tetrahydro-1,8-naphthyridin-2-yl)ethoxy]-1H-pyrazol-3-yl}butanoic acid; 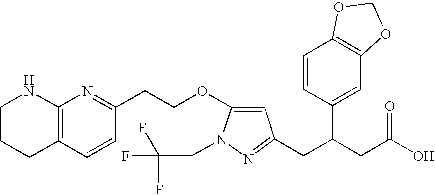
h) 3-(1,3-benzodioxol-5-yl)-4-[5-[2-(5,6,7,8-tetrahydro-1,8-naphthyridin-2-yl)ethoxy]-1-(2,2,2-trifluoroethyl)-1H-pyrazol-3-yl]butanoic acid; 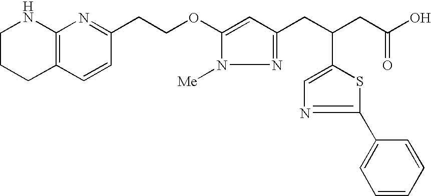
i) 4-{1-methyl-5-[2-(5,6,7,8-tetrahydro-1,8-naphthyridin-2-yl)ethoxy]-1H-pyrazol-3-yl}-3-(2-phenyl-1,3-thiazol-5-yl)butanoic acid; 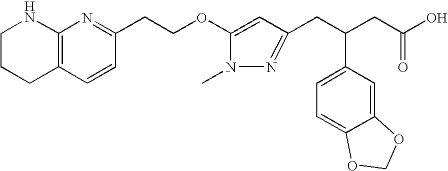
j) 3-(1,3-benzodioxol-5-yl)-4-(1-methyl-5-{2-[6-(methylamino)pyridin-2-yl]ethoxy}-1H-pyrazol-3-yl)butanoic acid; 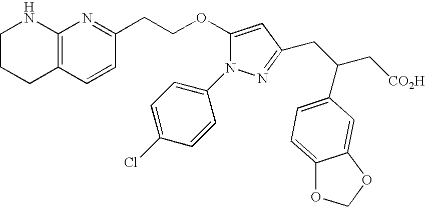
k) 3-(1,3-benzodioxol-5-yl)-4-{1-(4-chlorophenyl)-5-[2-(5,6,7,8-tetrahydro-1,8-naphthyridin-2-yl)ethoxy]-1H-pyrazol-3-yl}butanoic acid; 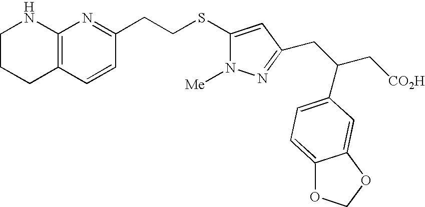
l) 3-(1,3-benzodioxol-5-yl)-4-(1-methyl-5-{[2-(5,6,7,8-tetrahydro-1,8-naphthyridin-2-yl)ethyl]thio}-1H-pyrazol-3-yl)butanoic acid; 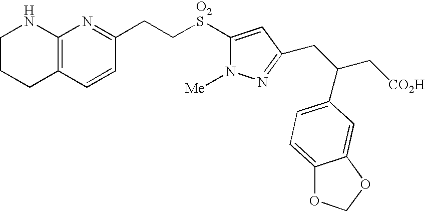
m) 3-(1,3-benzodioxol-5-yl)-4-(1-methyl-5-{[2-(5,6,7,8-tetrahydro-1,8-naphthyridin-2-yl)ethyl]sulfonyl}-1H-pyrazol-3-yl)butanoic acid; 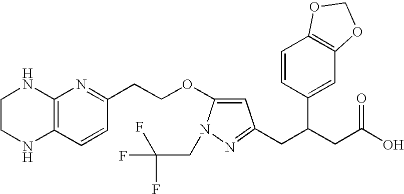
n) 3-(1,3-benzodioxol-5-yl)-4-[5-[2-(1,2,3,4-tetrahydropyrido[2,3-b]pyrazin-6-yl)ethoxy]-1-(2,2,2-trifluoroethyl)-1H-pyrazol-3-yl]butanoic acid; 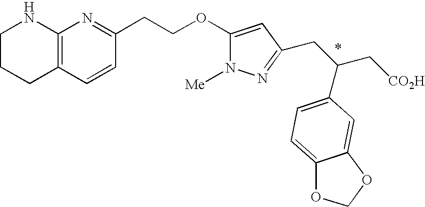
o) (S)-3-(1,3-benzodioxol-5-yl)-4-{1-methyl-5-[2-(5,6,7,8-tetrahydro-1,8-naphthyridin-2-yl)ethoxy]-1H-pyrazol-3-yl}butanoic acid; 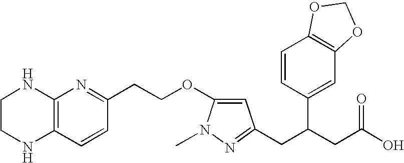
p) 3-(1,3-benzodioxol-5-yl)-4-{1-methyl-5-[2-(1,2,3,4-tetrahydropyrido[2,3-b]pyrazin-6-yl)ethoxy]-1H-pyrazol-3-yl}butanoic acid; 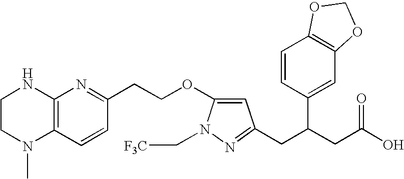
q) 3-(1,3-benzodioxol-5-yl)-4-[5-[2-(1-methyl-1,2,3,4-tetrahydropyrido[2,3-b]pyrazin-6-yl)ethoxy]-1-(2,2,2-trifluoroethyl)-1H-pyrazol-3-yl]butanoic acid; 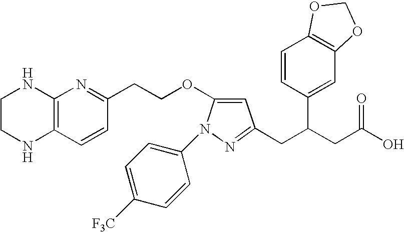
r) 3-(1,3-benzodioxol-5-yl)-4-{t5-[2-(5,6, 7,8-tetrahydro-1,8-naphthyridin-2-yl)ethoxy]-1-[4-(trifluoromethyl)phenyl]-H-pyrazol-3-yl}butanoic acid; 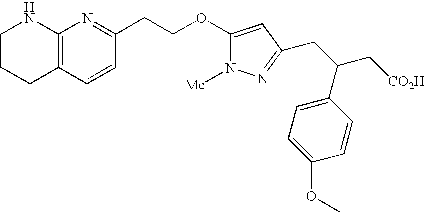
s) 3-(6-methoxypyridin-3-yl)-4-{1-methyl-5-[2-(5,6,7,8-tetrahydro-1,8-naphthyridin-2-yl)ethoxy]-11H-pyrazol-3-yl}butanoic acid; 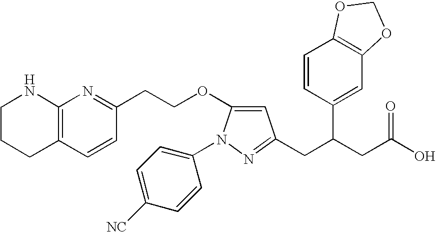
t) 3-(1,3-benzodioxol-5-yl)-4-{1-(4-cyanophenyl)-5-[2-(5,6,7,8-tetrahydro-1,8-naphthyridin-2-yl)ethoxy]-1H-pyrazol-3-yl}butanoic acid; 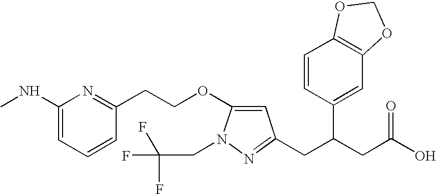
u) 3-(1,3-benzodioxol-5-yl)-4-[5-{2-[6-(methylamino)pyridin-2-yl]ethoxy}-1-(2,2,2-trifluoroethyl)-1H-pyrazol-3-yl]butanoic acid; 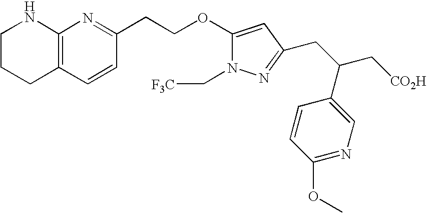
v) 3-(6-methoxypyridin-3-yl)-4-[5-[2-(5,6,7,8-tetrahydro-1,8-naphthyridin-2-yl)ethoxy]-1-(2,2,2-trifluoroethyl)-1H-pyrazol-3-yl]butanoic acid; 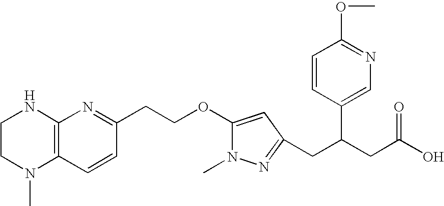
w) 3-(6-methoxypyridin-3-yl)-4-{1-methyl-5-[2-(1-methyl-1,2,3,4-tetrahydropyrido[2,3-b]pyrazin-6-yl)ethoxy]-1H-pyrazol-3-60 yl}butanoic acid; 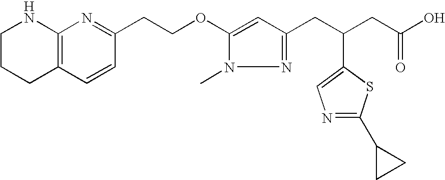
x) 3-(2-cyclopropyl-1,3-thiazol-5-yl)-4-{1-methyl-5-[2-(5,6,7,8-tetrahydro-1,8-naphthyridin-2-yl)ethoxy]-1H-pyrazol-3-yl}butanoic acid; 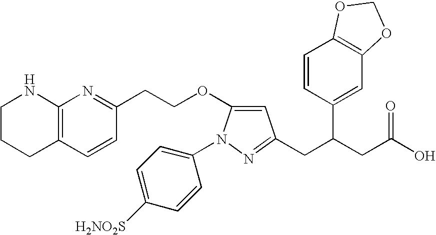
y) 4-{1-[4-(aminosulfonyl)phenyl]-5-[2-(5,6,7,8-tetrahydro-1,8-naphthyridin-2-yl)ethoxy]-1H-pyrazol-3-yl}-3-(1,3-benzodioxol-5-yl)butanoic acid; 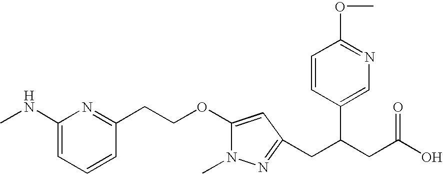
z) 3-(6-methoxypyridin-3-yl)-4-(1-methyl-5-{2-[6-(methylamino)pyridin-2-yl]ethoxy}-1H-pyrazol-3-yl)butanoic acid; 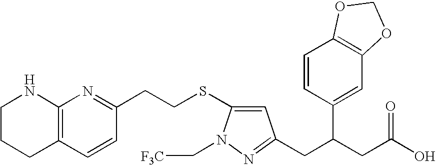
aa) 3-(1,3-benzodioxol-5-yl)-4-[5-{[2-(5,6,7,8-tetrahydro-1,8-naphthyridin-2-yl)ethyl]thio}-1-(2,2,2-trifluoroethyl)-1H-pyrazol-3-yl]butanoic acid; 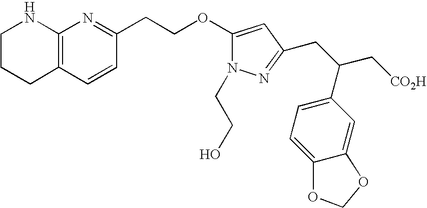
bb) 3-(1,3-benzodioxol-5-yl)-4-{1-(2-hydroxyethyl)-5-[2-(5,6,7,8-tetrahydro-1,8-naphthyridin-2-yl)ethoxy]-1H-pyrazol-3-yl}butanoic acid; 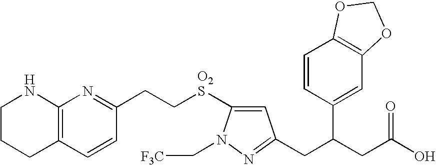
cc) 3-(1,3-benzodioxol-5-yl)-4-[5-{[2-(5,6,7,8-tetrahydro-1,8-naphthyridin-2-yl)ethyl]sulfonyl}-1-(2,2,2-trifluoroethyl)-1H-pyrazol-3-yl]butanoic acid; 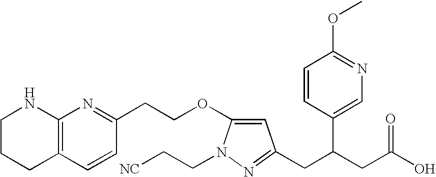
dd) 4-{1-(2-cyanoethyl)-5-[2-(5,6,7,8-tetrahydro-1,8-naphthyridin-2-yl)ethoxy]-1H-pyrazol-3-yl}-3-(6-methoxypyridin-3-yl)butanoic acid; 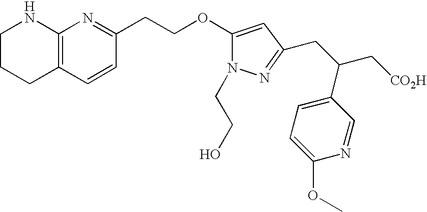
ee) 4-{1-(2-hydroxyethyl)-5-[2-(5,6,7,8-tetrahydro-1,8-naphthyridin-2-yl)ethoxy]-1H-pyrazol-3-yl}-3-(6-methoxypyridin-3-yl)butanoic acid; and 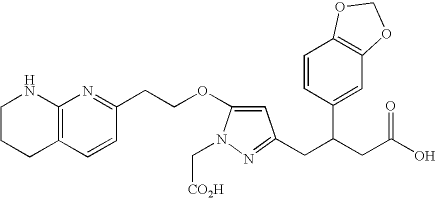
ff) 3-(1,3-benzodioxol-5-yl)-4-{1-(carboxymethyl)-5-[2-(5,6,7,8-tetra hydro-1,8-naphthyridin-2-yl)ethoxy]-1H-pyrazol-3-yl}butanoic acid.

9. A pharmaceutical composition comprising a therapeutically effective amount of a compound of claim 1 and a pharmaceutically acceptable carrier.

10. A method for the treatment or prevention of conditions mediated by the α_{V}β₃ integrin in a mammal in need of such treatment, the method comprising administering to the subject a therapeutically effective amount of a compound of claim 1.

11. The method according to claim 10 wherein the condition treated is selected from the group consisting of tumor metastasis, solid tumor growth, angiogenesis, osteoporosis, humoral hypercalcemia of malignancy, smooth muscle cell migration, restenosis, atheroscelorosis, macular degeneration, retinopathy, and arthritis.

12. A method for the treatment or prevention of conditions mediated by the α_{V}β₅ integrin in a mammal in need of such treatment, the method comprising administering to the subject a therapeutically effective amount of a compound of claim 1.

13. The method according to claim 12 wherein the condition treated is selected from the group consisting of tumor metastasis, solid tumor growth, angiogenesis, osteoporosis, humoral hypercalcemia of malignancy, smooth muscle cell migration, restenosis, atheroscelorosis, macular degeneration, retinopathy, and arthritis.
